(19) United States
(12) Patent Application Publication
(10) Pub. No.: US 2013/0053381 A1
(43) Pub. Date: Feb. 28, 2013

(54) 2,4-DIOXO-1,4-DIHYDRO-2H-QUINAZOLIN-3-YL-SULFONAMIDE DERIVATIVES

(71) Applicants: Yves Auberson, Allschwil (CH); David Carcache, Binningen (CH); Andreas Lerchner, Binningen (CH); Joachim Nozulak, Heitersheim (DE)

(72) Inventors: Yves Auberson, Allschwil (CH); David Carcache, Binningen (CH); Andreas Lerchner, Binningen (CH); Joachim Nozulak, Heitersheim (DE)

(73) Assignee: NOVARTIS AG

(21) Appl. No.: 13/638,082

(22) PCT Filed: May 18, 2011
(86) PCT No.: PCT/EP2011/058068

§ 371 Date: Sep. 28, 2012

Related U.S. Application Data
(60) Provisional application No. 61/346,577, filed on May 20, 2010.

(51) Int. Cl. C07D 403/10; C07D 239/96; C07D 413/14; A61K 31/5377; C07D 405/10; A61P 21/00; A61P 25/28; A61P 25/00; A61P 25/16; A61P 25/18; A61P 1/08; A61P 27/16; A61K 31/517; A61P 25/08

(52) U.S. Cl. 514/234.5; 544/284; 514/266.23; 544/285; 514/266.3; 544/119; 514/266.2; 514/266.24

(57) ABSTRACT

The invention relates to a 2,4-dioxo-1,4-dihydro-2H-quinazolin-3-yl-sulfonamide derivative being (A) a compound of the formula (I) in which the substituents are as defined in the specification; in free form or in salt form; or (B) a compound selected from a certain group of 2,4-dioxo-1,4-dihydro-2H-quinazolin-3-yl-sulfonamide derivatives disclosed in the specification; in free form or in salt form; to their preparation, to their use as medicament and to medicaments comprising them. In a first aspect, the invention provides prodrugs of AMPA receptor antagonists which potentially useful in the treatment of a wide range of disorders, particularly epilepsy.

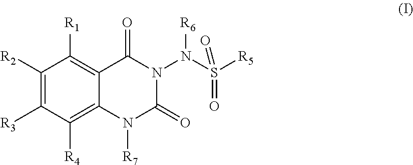

The invention relates to 2,4-dioxo-1,4-dihydro-2H-quinazolin-3-yl-sulfonamide derivatives, to their preparation, to their use as medicaments and to medicaments comprising them.

AMPA receptors are tetramers composed of four subunits GluR₂ to GluR₄, assembled either as homo- or heteromeric complexes which form a channel permeable for Na⁺ and K⁺. If the subunit GluR₂ is absent in the tetramer, the receptor is also permeable for Ca²⁺. Upon binding of two molecules of the endogenous transmitter glutamate, AMPA receptors undergo conformational changes leading to rapid activation of the channel, allowing an influx of cations into cells.

Regions with expressions of AMPA receptors include the hippocampus, superficial layers of the cerebral cortex deep in cortical layers, caudate putamen, diencephalon, midbrain, brainstem and cerebellum. AMPA receptors are also be found in the heart, ovary, uterus, kidney, testis, gastrointestinal tissue, lungs, spleen, bone, bone marrow, mast cells, inflammatory, and tumor cells.

Pathologies, disorders or clinical conditions where an altered AMPA receptor function or AMPA receptor mediated neuronal damage is believed to be underlying are, e.g. epilepsy, neurodegenerative disorders, such as multiple sclerosis, amyotrophic lateral sclerosis, Rasmussen's encephalitis, Parkinson's Disease, Huntington's Disease or Alzheimers Disease, schizophrenia, emesis, tinnitus or muscle spasticity.

Compounds being able to block AMPA-binding sites are described, for example, in WO199519346, WO199708155, WO2006108591 and WO2006010591.

There is a need to provide drugs or prodrugs that act in-vivo as AMPA receptor antagonists and are good drug candidates.

In a first aspect, the invention provides prodrugs of AMPA receptor antagonists which are potentially useful in the treatment of a wide range of disorders, particularly epilepsy.

In said first aspect, the invention relates to a 2,4-dioxo-1,4-dihydro-2H-quinazolin-3-yl-sulfonamide derivative being

(A) a compound of the formula I

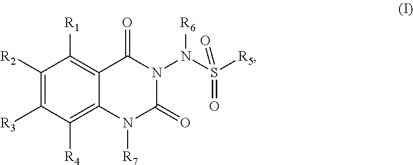

wherein
R₁ is hydrogen, halogen, C₁₋₄alkyl, C₁₋₄halogenalkyl, C₃₋₄cycloalkyl or C₃₋₄halogencycloalkyl;
R₂ is a group selected from

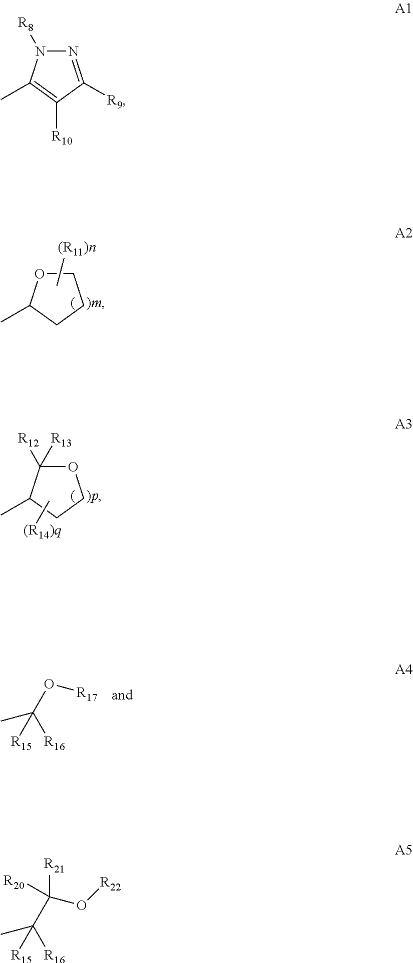

R₈ is hydrogen; hydroxy; C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the nitrogen atom of the group A1 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the nitrogen atom of the group A1 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₉ and R₁₀ independently are hydrogen or fluoro;
m is 1 or 2;
n is 0, 1, 2 or 3;
R₁₁ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A2 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A2 or via a C₁₋₂alkylene or an oxygen, and wherein the phenyl may be substituted by halogen, hydroxy or C₁₋₄alkyl; C₁₋₆alkoxy; or C₁₋₆haloalkoxy;
p is 1 or 2;
q is 0, 1, 2 or 3;
R₁₂ is hydrogen, halogen, C₁₋₃alkyl, C₁₋₃halogenalkyl or cyclopropyl; and R₁₃ is hydrogen;
or R₁₂ and R₁₃ are independently halogen or methyl;
or R₁₂ and R₁₃ together with the carbon atom to which they are bound form a cyclopropyl;
R₁₄ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A3 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A3 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₁₅ and R₁₆ independently are hydrogen; halogen; cyano; C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A4 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A4 or via a C₁₋₂alkylene or an oxygen, and wherein the phenyl may be substituted by halogen, hydroxy or C₁₋₄alkyl; C₁₋₆alkoxy; or C₁₋₆haloalkoxy;
or R₁₅ and R₁₆ together with the carbon atom to which they are bound form a C₃₋₆cycloalkyl;
R₁₇ is C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the oxygen atom of the group A4 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A4 or via a C₁₋₂alkylene, and wherein the phenyl may be substituted by halogen, hydroxy or C₁₋₄alkyl;
R₁₈ and R₁₉ independently are hydrogen; halogen; cyano; C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A5 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A5 or via a C₁₋₂alkylene or an oxygen, and wherein the phenyl may be substituted by halogen, hydroxy or C₁₋₄alkyl; C₁₋₆alkoxy; or C₁₋₆haloalkoxy; or R₁₈ and R₁₉ together with the carbon atom to which they are bound form a C₃₋₆cycloalkyl;
R₂₀ is hydrogen, halogen, C₁₋₃alkyl, C₁₋₃halogenalkyl or cyclopropyl; and R₂₁ is hydrogen;
or R₂₀ and R₂₁ are independently halogen or methyl;
or R₂₀ and R₂₁ together with the carbon atom to which they are bound form a cyclopropyl;
or R₁₈ and R₂₀ together with the adjacent carbon atoms to which they are bound form a C₃₋₆cycloalkyl; and R₁₉ and R₂₁ are hydrogen;
R₂₂ is C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the oxygen atom of the group A5 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A5 or via a C₁₋₂alkylene, and wherein the phenyl may be substituted by halogen, hydroxy or C₁₋₄alkyl;
R₃ is C₁₋₄halogenalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃, C₃₋₆cycloalkylcarbonyl which may be substituted once or more than once by R₂₄, phenylcarbonyl which may be substituted once or more than once by R₂₅, C₃₋₆cycloalkyl-C₁₋₂alkylcarbonyl which may be substituted once or more than once by R₂₆, phenyl-C₁₋₂alkylcarbonyl which may be substituted once or more than once by R₂₇; C₁₋₁₀alkoxycarbonyl which may be substituted once or more than once by R₂₈, or C₃₋₆cycloalkoxycarbonyl which may be substituted once or more than once by R₂₉, phenyloxycarbonyl which may be substituted once or more than once by R₃₀, C₃₋₆cycloalkyl-C₁₋₂alkoxycarbonyl which may be substituted once or more than once by R₃₁, phenyl-C₁₋₂alkoxycarbonyl which may be substituted once or more than once by R₃₂;
R₇ is hydrogen, C₁₋₁₀alkylcarbonyl which may be substituted once or more than once by R₃₃, C₃₋₆cycloalkylcarbonyl which may be substituted once or more than once by R₃₄, phenylcarbonyl which may be substituted once or more than once by R₃₅, C₃₋₆cycloalkyl-C₁₋₂alkylcarbonyl which may be substituted once or more than once by R₃₆, phenyl-C₁₋₂alkylcarbonyl which may be substituted once or more than once by R₃₇; C₁₋₁₀alkoxycarbonyl which may be substituted once or more than once by R₃₈, or C₃₋₆cycloalkoxycarbonyl which may be substituted once or more than once by R₃₉, phenyloxycarbonyl which may be substituted once or more than once by R₄₀, C₃₋₆cycloalkyl-C₁₋₂alkoxycarbonyl which may be substituted once or more than once by R₄₁, phenyl-C₁₋₂alkoxycarbonyl which may be substituted once or more than once by R₄₂;
each R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁ and R₄₂ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl;
in free form or in salt form; or
(B) a compound selected from the group consisting of
4-[(6-Imidazol-1-yl-2,4-dioxo-7-trifluoromethyl-1,4-dihydro-2H-quinazolin-3-yl)-methanesulfonyl-amino]-4-oxo-butyric acid ethyl ester;
N-(6-Imidazol-1-yl-2,4-dioxo-7-trifluoromethyl-1,4-dihydro-2H-quinazolin-3-yl)-N-pentanoyl-methanesulfonamide;
N-Acetyl-N-(6-imidazol-1-yl-2,4-dioxo-7-trifluoromethyl-1,4-dihydro-2H-quinazolin-3-yl)-methanesulfonamide;
N-(6-Imidazol-1-yl-2,4-dioxo-7-trifluoromethyl-1,4-dihydro-2H-quinazolin-3-yl)-N-propionyl-methanesulfonamide;
N-(6-Imidazol-1-yl-2,4-dioxo-7-trifluoromethyl-1,4-dihydro-2H-quinazolin-3-yl)-N-isobutyryl-methanesulfonamide;
N-(6-Imidazol-1-yl-2,4-dioxo-7-trifluoromethyl-1,4-dihydro-2H-quinazolin-3-yl)-N-(3-methyl-butyryl)-methanesulfonamide;
N-Cyclopentanecarbonyl-N-(6-imidazol-1-yl-2,4-dioxo-7-trifluoromethyl-1,4-dihydro-2H-quinazolin-3-yl)-methanesulfonamide;
N-Hexanoyl-N-(6-imidazol-1-yl-2,4-dioxo-7-trifluoromethyl-1,4-dihydro-2H-quinazolin-3-yl)-methanesulfonamide;
N-Butyryl-N-(6-imidazol-1-yl-2,4-dioxo-7-trifluoromethyl-1,4-dihydro-2H-quinazolin-3-yl)-methanesulfonamide;
Methanesulfonyl-[6-(2-methyl-imidazol-1-yl)-2,4-dioxo-7-trifluoromethyl-1,4-dihydro-2H-quinazolin-3-yl]-carbamic acid ethyl ester;
N-Butyryl-N-[6-(2-methyl-imidazol-1-yl)-2,4-dioxo-7-trifluoromethyl-1,4-dihydro-2H-quinazolin-3-yl]-methanesulfonamide;
Methanesulfonyl-[6-(2-methyl-imidazol-1-yl)-2,4-dioxo-7-trifluoromethyl-1,4-dihydro-2H-quinazolin-3-yl]-carbamic acid propyl ester;
Methanesulfonyl-[6-(2-methyl-imidazol-1-yl)-2,4-dioxo-7-trifluoromethyl-1,4-dihydro-2H-quinazolin-3-yl]-carbamic acid isobutyl ester;
N-(2,4-Dioxo-6-pyrrol-1-yl-7-trifluoromethyl-1,4-dihydro-2H-quinazolin-3-yl)-N-propionyl-methanesulfonamide;
N-Butyryl-N-(2,4-dioxo-6-pyrrol-1-yl-7-trifluoromethyl-1,4-dihydro-2H-quinazolin-3-yl)-methanesulfonamide;
Acetic acid 2-[(2,4-dioxo-6-pyrrol-1-yl-7-trifluoromethyl-1,4-dihydro-2H-quinazolin-3-yl)-methanesulfonyl-amino]-2-oxo-ethyl ester;
(2,4-Dioxo-6-pyrrol-1-yl-7-trifluoromethyl-1,4-dihydro-2H-quinazolin-3-yl)-methanesulfonyl-carbamic acid methyl ester;
(2,4-Dioxo-6-pyrrol-1-yl-7-trifluoromethyl-1,4-dihydro-2H-quinazolin-3-yl)-methanesulfonyl-carbamic acid ethyl ester;
N-(2,4-Dioxo-6-[1,2,4]triazol-4-yl-7-trifluoromethyl-1,4-dihydro-2H-quinazolin-3-yl)-N-(3-methyl-butyryl)-methanesulfonamide;
N-(2,4-Dioxo-6-[1,2,4]triazol-4-yl-7-trifluoromethyl-1,4-dihydro-2H-quinazolin-3-yl)-N-hexanoyl-methanesulfonamide;
N-(2,4-Dioxo-6-[1,2,4]triazol-4-yl-7-trifluoromethyl-1,4-dihydro-2H-quinazolin-3-yl)-N-pentanoyl-methanesulfonamide;
N-Butyryl-N-(2,4-dioxo-6-[1,2,4]triazol-4-yl-7-trifluoromethyl-1,4-dihydro-2H-quinazolin-3-yl)-methanesulfonamide;
N-Hexanoyl-N-[6-(1-hydroxy-propyl)-2,4-dioxo-7-trifluoromethyl-1,4-dihydro-2H-quinazolin-3-yl]-methanesulfonamide;
Methanesulfonyl-[6-(1-hydroxy-propyl)-2,4-dioxo-7-trifluoromethyl-1,4-dihydro-2H-quinazolin-3-yl]-carbamic acid 2-methoxy-ethyl ester;
N-Butyryl-N-[6-(1-hydroxy-propyl)-2,4-dioxo-7-trifluoromethyl-1,4-dihydro-2H-quinazolin-3-yl]-methanesulfonamide;
N-[6-(1-hydroxy-propyl)-2,4-dioxo-7-trifluoromethyl-1,4-dihydro-2H-quinazolin-3-yl]-N-isobutyryl-methanesulfonamide; and
N-[6-(1-hydroxy-propyl)-2,4-dioxo-7-trifluoromethyl-1,4-dihydro-2H-quinazolin-3-yl]-N-pentanoyl-methanesulfonamide; in free form or in salt form.

The term “compounds of the invention” comprise compounds of formula (I) and the 27 individual 2,4-dioxo-1,4-dihydro-2H-quinazolin-3-yl-sulfonamide derivatives disclosed above (first derivative disclosed being 4-[(6-Imidazol-1-yl-2,4-dioxo-7-trifluoromethyl-1,4-dihydro-2H-quinazolin-3-yl)-methanesulfonyl-amino]-4-oxo-butyric acid ethyl ester; last derivative disclosed being N-[6-(1-hydroxy-propyl)-2,4-dioxo-7-trifluoromethyl-1,4-dihydro-2H-quinazolin-3-yl]-N-pentanoyl-methanesulfonamide).

The term “prodrugs of the invention” comprise compounds of formula (I) and the 27 individual 2,4-dioxo-1,4-dihydro-2H-quinazolin-3-yl-sulfonamide derivatives disclosed above (first derivative disclosed being 4-[(6-Imidazol-1-yl-2,4-dioxo-7-trifluoromethyl-1,4-dihydro-2H-quinazolin-3-yl)-methanesulfonyl-amino]-4-oxo-butyric acid ethyl ester; last derivative disclosed being N-[6-(1-hydroxy-propyl)-2,4-dioxo-7-trifluoromethyl-1,4-dihydro-2H-quinazolin-3-yl]-N-pentanoyl-methanesulfonamide).

In particular, preferred prodrugs of the invention should be well absorbed from the gastrointestinal tract, be transformed into the parent compound (or active principle, being the compound that in-vivo acts as AMPA receptor antagonist), the parent compound should be sufficiently metabolically stable and possess favorable pharmacokinetic properties.

Further preferred prodrugs of the invention lead to an oral bioavailability of the parent compound which is comparable to the use of the parent compound as administered drug

Further preferred prodrugs of the invention increase the oral bioavailability of the parent compound compared to the use of the parent compound as administered drug. Said increased oral bioavailability may manifest itself in different ways: (i) a biological effect may be achieved after oral administration when the parent compound is ineffective upon oral administration, (ii) an earlier onset of action upon oral administration, (iii) a lower dose needed to achieve the same effect, (iv) a higher effect achieved by the same dose or (v) a prolonged action at the same dose.

Further preferred prodrugs of the invention are transformed into parent compounds which in-vivo bind potently to AMPA receptors whilst showing little affinity for other receptors.

Some prodrugs of the invention are transformed into parent compounds which also show antagonistic activity at kainate receptors. As migraine is a condition where an overactivity of kainate receptors is implicated, said prodrugs are suitable to treat migraine. Besides such dual activity, showing little affinity for other receptors is a preferred feature.

Further preferred prodrugs of the invention—when the active principle is targeted against receptors in the central nervous system—are transformed into parent compounds that cross the blood brain barrier freely.

Further preferred prodrugs of the invention—when the active principle is targeted selectively against receptors in the peripheral nervous system—are transformed into parent compounds that do not cross the blood brain barrier.

Prodrugs, parent compounds and released pro-moieties should be non-toxic and demonstrate few side-effects.

Furthermore, the ideal prodrug of the invention will be able to exist in a physical form that is stable, non-hygroscopic and easily formulated.

Unless indicated otherwise, the expressions used in this invention have the following meaning:

“Alkyl” represents a straight-chain or branched-chain alkyl group, for example, methyl, ethyl, n- or iso-propyl, n-, iso-, sec- or tert-butyl, n-pentyl, n-hexyl; C₁₋₁₀alkyl preferably represents a straight-chain or branched-chain C₁₋₆alkyl, more preferably a straight-chain or branched-chain C₁₋₄alkyl with particular preference given to methyl, ethyl, n-propyl, iso-propyl and tert-butyl.

Each alkyl part of “alkoxy”, “halogenalkyl” and so on shall have the same meaning as described in the above-mentioned definition of “alkyl”, especially regarding linearity and preferential size.

“C₃₋₆cycloalkyl” represents a saturated alicyclic moiety having from three to six carbon atoms. This term refers to groups such as cyclopropyl, cyclobutyl, cyclopentyl and cyclohexyl.

A substituent being substituted “once or more than once”, for example as defined for R₈, is preferably substituted by one to three substituents.

Halogen is generally fluorine, chlorine, bromine or iodine; preferably fluorine, chlorine or bromine. Halogenalkyl groups are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl; preferably —CF₃, —CHF₂, —CH₂F, —CHF—CH₃, —CF₂CH₃, or —CH₂CF₃.

In the context of the invention, the definition of R₅ as a “three- to seven-membered monocyclic ring system which may be aromatic, saturated or unsaturated non-aromatic and which may contain from 1 to 4 hetero atoms selected from nitrogen, oxygen and sulfur” encompasses a C₆-aromatic hydrocarbon group; a five- to six-membered heterocyclic aromatic ring system; a three- to seven-membered monocyclic non-aromatic hydrocarbon group and a non-aromatic heterocyclic ring system of the same size.

Preferably, but also depending on substituent definition, “five- to six-membered heterocyclic aromatic ring systems” consist of 5 to 6 ring atoms of which 1-3 ring atoms are hetero atoms.

Examples of heterocyclic ring systems are: imidazo[2,1-b]thiazole, pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazolidine, isothiazole, isothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pteridine, and the corresponding benz-annelated heterocycles, e.g. indole, isoindole, coumarin, isoquinoline, quinoline and the like. Preferred heterocycles are: imidazo[2,1-b]thiazole, oxazole, isoxazole, thiazole, isothiazole, triazole, pyrrole, furane, tetrahydrofurane, pyridine, pyrimidine, imidazole or pyrazole.

The compounds of the invention may exist in optically active form or in form of mixtures of optical isomers, e.g. in form of racemic mixtures or diastereomeric mixtures. In particular, asymmetrical carbon atom(s) may be present in the compounds of the invention and their salts. All optical isomers and their mixtures, including the racemic mixtures, are embraced by the invention.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture.

The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. The compounds described herein contain one or more asymmetric centers and may thus give rise to enantiomers, diastereomers, and other stereoisomeric forms that may be defined, in terms of absolute stereochemistry, as (R)- or (S)-. The invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

Any asymmetric atom (e.g. carbon or the like) of the compound(s) of the invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Depending on substituent definition, compounds of the invention may occur in various tautomeric forms. All tautomeric forms of the compounds of the invention are embraced by the invention.

Compounds of the invention may exist in free form or as a salt. In this specification, unless otherwise indicated, language such as “compound of formula I” is to be understood as embracing the compounds in any form, for example free or acid addition salt form. Salts which are unsuitable for pharmaceutical uses but which can be employed, for example, for the isolation or purification of free compounds of the invention, such as picrates or perchlorates, are also included. For therapeutic use, only pharmaceutically acceptable salts or free compounds are employed (where applicable in the form of pharmaceutical preparations), and are therefore preferred. Salts are preferably physiologically acceptable salts, formed by the addition of an acid.

As used herein, the term “pharmaceutically acceptable salts” refers to salts that retain the biological effectiveness and properties of the compounds of this invention and, which typically are not biologically or otherwise undesirable. The compounds of the invention may be capable of forming acid salts by virtue of the presence of suitable groups, such as amino groups.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

The pharmaceutically acceptable salts of the invention can be synthesized from a parent compound by conventional chemical methods. Generally, such salts can be prepared by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The invention includes all pharmaceutically acceptable isotopically-labeled compounds of the invention, e.g. compounds of formula (I), wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio. Examples of isotopes suitable for inclusion in the compounds of the invention comprises isotopes of hydrogen, such as ²H. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of the invention with the co-crystal former under crystallization conditions and isolating co-crystals thereby formed. Suitable co-crystal formers include those described in WO 2004/078163. Hence the invention further provides co-crystals comprising a compound of formula (I).

Compounds of the invention are either obtained in the free form or as a salt thereof.

Furthermore, the compounds of the invention, including their salts, can also be obtained in the form of their hydrates, or include other solvents used for their crystallization.

Preferred substituents, preferred ranges of numerical values or preferred ranges of the radicals present in compounds of the formula I and the corresponding intermediate compounds are defined below. The definition of the substituents applies to the end-products as well as to the corresponding intermediates. The definitions of the substituents may be combined at will, e.g. preferred substituents R¹ and particularly preferred substituents R².

In especially preferred embodiments, the invention relates to one or more than one of the compounds of the formula I mentioned in the Examples hereinafter, in free form or in salt form.

One class of compounds of the invention relates to a compound of the formula I, wherein R₁ is hydrogen, halogen, C₁₋₄alkyl, C₁₋₄halogenalkyl, C₃₋₄cycloalkyl or C₃₋₄halogencycloalkyl; R₂ is a group selected from

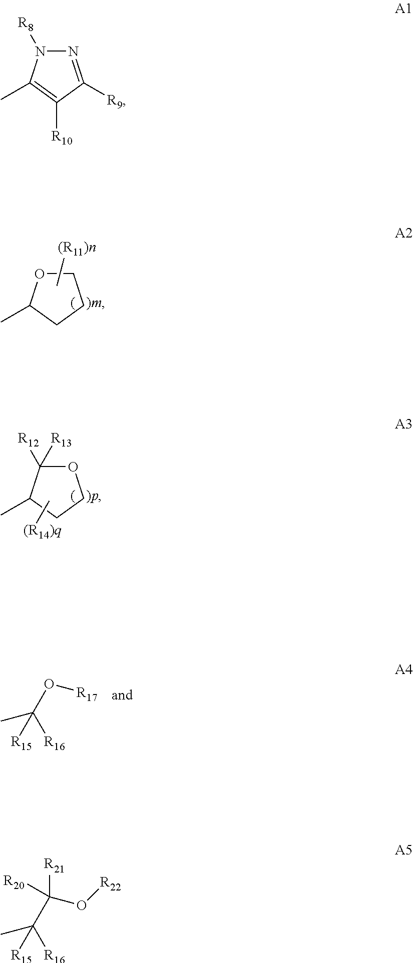

R₈ is hydrogen; hydroxy; C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the nitrogen atom of the group A1 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the nitrogen atom of the group A1 or via a C₁₋₂alkylene or an oxygen, and wherein the phenyl may be substituted by halogen, hydroxy or C₁₋₄alkyl; C₁₋₆alkoxy; or C₁₋₈haloalkoxy;
R₉ and R₁₀ independently are hydrogen or fluoro;
m is 1 or 2;
n is 0, 1, 2 or 3;
R₁₁ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A2 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A2 or via a C₁₋₂alkylene or an oxygen, and wherein the phenyl may be substituted by halogen, hydroxy or C₁₋₄alkyl; C₁₋₆alkoxy; or C₁₋₆haloalkoxy;
p is 1 or 2;
q is 0, 1, 2 or 3;
R₁₂ is hydrogen, halogen, C₁₋₃alkyl, C₁₋₃halogenalkyl or cyclopropyl; and R₁₃ is hydrogen; or R₁₂ and R₁₃ are independently halogen or methyl; or R₁₂ and R₁₃ together with the carbon atom to which they are bound form a cyclopropyl;
R₁₄ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A3 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A3 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₁₅ and R₁₆ independently are hydrogen; halogen; cyano; C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A4 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A4 or via a C₁₋₂alkylene or an oxygen, and wherein the phenyl may be substituted by halogen, hydroxy or C₁₋₄alkyl; C₁₋₆alkoxy; or C₁₋₆haloalkoxy; or R₁₅ and R₁₆ together with the carbon atom to which they are bound form a C₃₋₆cycloalkyl;
R₁₇ is C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the oxygen atom of the group A4 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A4 or via a C₁₋₂alkylene, and wherein the phenyl may be substituted by halogen, hydroxy or C₁₋₄alkyl;
R₁₈ and R₁₉ independently are hydrogen; halogen; cyano; C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A5 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A5 or via a C₁₋₂alkylene or an oxygen, and wherein the phenyl may be substituted by halogen, hydroxy or C₁₋₄alkyl; C₁₋₆alkoxy; or C₁₋₆haloalkoxy; or R₁₈ and R₁₉ together with the carbon atom to which they are bound form a C₃₋₆cycloalkyl;
R₂₀ is hydrogen, halogen, C₁₋₃alkyl, C₁₋₃halogenalkyl or cyclopropyl; and R₂₁ is hydrogen;
or R₂₀ and R₂₁ are independently halogen or methyl;
or R₂₀ and R₂₁ together with the carbon atom to which they are bound form a cyclopropyl;
or R₁₈ and R₂₀ together with the adjacent carbon atoms to which they are bound form a C₃₋₆cycloalkyl; and R₁₉ and R₂₁ are hydrogen;
R₂₂ is C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the oxygen atom of the group A5 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A5 or via a C₁₋₂alkylene, and wherein the phenyl may be substituted by halogen, hydroxy or C₁₋₄alkyl;
R₃ is C₁₋₄halogenalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃, C₃₋₆cycloalkylcarbonyl which may be substituted once or more than once by R₂₄, phenylcarbonyl which may be substituted once or more than once by R₂₅, C₃₋₆cycloalkyl-C₁₋₂alkylcarbonyl which may be substituted once or more than once by R₂₆, phenyl-C₁₋₂alkylcarbonyl which may be substituted once or more than once by R₂₇; C₁₋₁₀alkoxycarbonyl which may be substituted once or more than once by R₂₈, or C₃₋₆cycloalkoxycarbonyl which may be substituted once or more than once by R₂₀, phenyloxycarbonyl which may be substituted once or more than once by R₃₀, C₃₋₆cycloalkyl-C₁₋₂alkoxycarbonyl which may be substituted once or more than once by R₃₁, phenyl-C₁₋₂alkoxycarbonyl which may be substituted once or more than once by R₃₂;
R₇ is hydrogen, C₁₋₁₀alkylcarbonyl which may be substituted once or more than once by R₃₃, C₃₋₆cycloalkylcarbonyl which may be substituted once or more than once by R₃₄, phenylcarbonyl which may be substituted once or more than once by R₃₅, C₃₋₆cycloalkyl-C₁₋₂alkylcarbonyl which may be substituted once or more than once by R₃₆, phenyl-C₁₋₂alkylcarbonyl which may be substituted once or more than once by R₃₇, C₁₋₁₀alkoxycarbonyl which may be substituted once or more than once by R₃₈, or C₃₋₆cycloalkoxycarbonyl which may be substituted once or more than once by R₃₉, phenyloxycarbonyl which may be substituted once or more than once by R₄₀, C₃₋₆cycloalkyl-C₁₋₂alkoxycarbonyl which may be substituted once or more than once by R₄₁, phenyl-C₁₋₂alkoxycarbonyl which may be substituted once or more than once by R₄₂;
each R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁ and R₄₂ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein R₁ is hydrogen.

One class of compounds of the invention relates to a compound of the formula I, wherein R₂ is a group A1.

One class of compounds of the invention relates to a compound of the formula I, wherein R₂ is a group A2, A3, A4 or A5.

One class of compounds of the invention relates to a compound of the formula I, wherein R₂ is a group A2 or A3.

One class of compounds of the invention relates to a compound of the formula I, wherein R₂ is a group A2.

One class of compounds of the invention relates to a compound of the formula I, wherein R₂ is a group A3.

One class of compounds of the invention relates to a compound of the formula I, wherein R₂ is a group A4 or A5.

One class of compounds of the invention relates to a compound of the formula I, wherein R₂ is a group A4.

One class of compounds of the invention relates to a compound of the formula I, wherein R₂ is a group A5.

One class of compounds of the invention relates to a compound of the formula I, wherein R₂ is a group A1; R₈ is C₁₋₆alkyl; and R₉ and R₁₀ independently are hydrogen or fluoro.

One class of compounds of the invention relates to a compound of the formula I, wherein R₂ is a group A2; m is 1 and n is O.

One class of compounds of the invention relates to a compound of the formula I, wherein R₂ is a group A3; p is 1; q is 0; and R₁₂ and R₁₃ are both hydrogen.

One class of compounds of the invention relates to a compound of the formula I, wherein R₂ is a group A4; R₁₅ is ethyl; R₁₆ is hydrogen and R₁₇ is methyl.

One class of compounds of the invention relates to a compound of the formula I, wherein R₂ is a group A5; R₁₈ is ethyl; R₁₀, R₂₀ and R₂₁ are hydrogen and R₂₂ is methyl.

One class of compounds of the invention relates to a compound of the formula I, wherein R₃ is C₁₋₄halogenalkyl or C₁₋₄alkyl.

One class of compounds of the invention relates to a compound of the formula I, wherein R₃ is halogenmethyl, especially trifluoromethyl.

One class of compounds of the invention relates to a compound of the formula I, wherein R₃ is C₁₋₄alkyl, especially isopropyl.

One class of compounds of the invention relates to a compound of the formula I, wherein R₄ is hydrogen.

One class of compounds of the invention relates to a compound of the formula I, wherein R₅ is C₁₋₄alkyl, especially methyl.

One class of compounds of the invention relates to a compound of the formula I, wherein R₆ is C₁₋₁₀alkylcarbonyl which may be substituted once or more than once by R₂₃ or C₁₋₁₀alkoxycarbonyl which may be substituted once or more than once by R₂₈.

One class of compounds of the invention relates to a compound of the formula I, wherein R₆ is C₁₋₁₀alkylcarbonyl which may be substituted once or more than once by R₂₃.

One class of compounds of the invention relates to a compound of the formula I, wherein R₆ is C₁₋₁₀alkoxycarbonyl which may be substituted once or more than once by R₂₈.

One class of compounds of the invention relates to a compound of the formula I, wherein R₇ is hydrogen, C₁₋₁₀alkylcarbonyl which may be substituted once or more than once by R₃₃ or C₁₋₁₀alkoxycarbonyl which may be substituted once or more than once by R₃₈.

One class of compounds of the invention relates to a compound of the formula I, wherein R₇ is hydrogen.

One class of compounds of the invention relates to a compound of the formula I, wherein R₇ is hydrogen or C₁₋₁₀alkylcarbonyl which may be substituted once or more than once by R₃₃.

One class of compounds of the invention relates to a compound of the formula I, wherein R₆ and R₇ are both C₁₋₁₀alkylcarbonyl which may be substituted once or more than once by R₃₃.

One class of compounds of the invention relates to a compound of the formula I, wherein each R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁ and R₄₂ independently is C₁₋₆alkoxy or C₁₋₆alkylthio.

One class of compounds of the invention relates to a compound of the formula I, wherein

R₁ is hydrogen, halogen, C₁₋₄alkyl, C₁₋₄halogenalkyl, C₃₋₄cycloalkyl or C₃₋₄halogencycloalkyl;
R₂ is a group A1;
R₈ is hydrogen; hydroxy; C₁₋₆alkyl; C₁₋₆haloalkyl; C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the nitrogen atom of the group A1 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the nitrogen atom of the group A1 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₉ and R₁₀ independently are hydrogen or fluoro;
R₃ is C₁₋₄halogenalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃, C₃₋₆cycloalkylcarbonyl which may be substituted once or more than once by R₂₄, phenylcarbonyl which may be substituted once or more than once by R₂₅, C₃₋₆cycloalkyl-C₁₋₂alkylcarbonyl which may be substituted once or more than once by R₂₆, phenyl-C₁₋₂alkylcarbonyl which may be substituted once or more than once by R₂₇; C₁₋₁₀alkoxycarbonyl which may be substituted once or more than once by R₂₈, or C₃₋₆cycloalkoxycarbonyl which may be substituted once or more than once by R₂₉; phenyloxycarbonyl which may be substituted once or more than once by R₃₀, C₃₋₆cycloalkyl-C₁₋₂alkoxycarbonyl which may be substituted once or more than once by R₃₁, phenyl-C₁₋₂alkoxycarbonyl which may be substituted once or more than once by R₃₂;
R₇ is hydrogen, C₁₋₁₀alkylcarbonyl which may be substituted once or more than once by R₃₃, C₃₋₆cycloalkylcarbonyl which may be substituted once or more than once by R₃₄, phenylcarbonyl which may be substituted once or more than once by R₃₅, C₃₋₆cycloalkyl-C₁₋₂alkylcarbonyl which may be substituted once or more than once by R₃₆, phenyl-C₁₋₂alkylcarbonyl which may be substituted once or more than once by R₃₇; C₁₋₁₀alkoxycarbonyl which may be substituted once or more than once by R₃₈, or C₃₋₆cycloalkoxycarbonyl which may be substituted once or more than once by R₃₉; phenyloxycarbonyl which may be substituted once or more than once by R₄₀, C₃₋₆cycloalkyl-C₁₋₂alkoxycarbonyl which may be substituted once or more than once by R₄₁, phenyl-C₁₋₂alkoxycarbonyl which may be substituted once or more than once by R₄₂;
each R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁ and R₄₂ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein

R₁ is hydrogen, halogen, C₁₋₄alkyl, C₁₋₄halogenalkyl, C₃₋₄cycloalkyl or C₃₋₄halogencycloalkyl;
R₂ is a group A1;
R₈ is C₁₋₆alkyl;
R₉ and R₁₀ independently are hydrogen or fluoro;
R₃ is C₁₋₄halogenalkyl or C₁₋₄alkyl;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃ or C₁₋₁₀alkoxycarbonyl which may be substituted once or more than once by R₂₈,
R₇ is hydrogen, C₁₋₁₀alkylcarbonyl which may be substituted once or more than once by R₃₃ or C₁₋₁₀alkoxycarbonyl which may be substituted once or more than once by R₃₈;
each R₂₃, R₂₈, R₃₃ and R₃₈ independently is C₁₋ₒalkoxy or, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein

R₁ is hydrogen, halogen, C₁₋₄alkyl, C₁₋₄halogenalkyl, C₃₋₄cycloalkyl or C₃₋₄halogencycloalkyl;
R₂ is a group A1;
R₈ is methyl;
R₉ and R₁₀ independently are hydrogen or fluoro;
R₃ is isopropyl, ethyl, trifluoromethyl, difluoromethyl or fluoromethyl;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃ or C₁₋₁₀alkoxycarbonyl which may be substituted once or more than once by R₂₈;
R₇ is hydrogen, C₁₋₁₀alkylcarbonyl which may be substituted once or more than once by R₃₃ or C₁₋₁₀alkoxycarbonyl which may be substituted once or more than once by R₃₈;
each R₂₃, R₂₈, R₃₃ and R₃₈ independently is C₁₋₆alkoxy or, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein

R₁ is hydrogen or halogen;
R₂ is a group A1;
R₈ is methyl;
R₉ and R₁₀ independently are hydrogen or fluoro;
R₃ is isopropyl, ethyl, trifluoromethyl, difluoromethyl or fluoromethyl;
R₄ is hydrogen or fluoro;
R₅ is C₁₋₄alkyl;
R₆ is linear C₁₋₆alkylcarbonyl which may be substituted once or more than once by R₂₃;
R₇ is hydrogen or linear C₁₋₆alkylcarbonyl which may be substituted once or more than once by R₃₃;
each R₂₃ and R₃₃ independently is C₁₋₄alkoxy or C₁₋₄alkylthio.

One class of compounds of the invention relates to a compound of the formula I, wherein

R₁ is hydrogen, halogen, C₁₋₄alkyl, C₁₋₄halogenalkyl, C₃₋₄cycloalkyl or C₃₋₄halogencycloalkyl;
R₂ is a group A1;
R₈ is methyl;
R₉ and R₁₀ independently are hydrogen or fluoro;
R₃ is ethyl, trifluoromethyl, difluoromethyl or fluoromethyl;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃ or C₁₋₁₀alkoxycarbonyl which may be substituted once or more than once by R₂₈;
R₇ is hydrogen, C₁₋₁₀alkylcarbonyl which may be substituted once or more than once by R₃₃ or C₁₋₁₀alkoxycarbonyl which may be substituted once or more than once by R₃₈;
each R₂₃, R₂₈, R₃₃ and R₃₈ independently is C₁₋₆alkoxy or, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein

R₁ is hydrogen or halogen;
R₂ is a group A1;
R₈ is methyl;
R₉ and R₁₀ independently are hydrogen or fluoro;
R₃ is ethyl, trifluoromethyl, difluoromethyl or fluoromethyl;
R₄ is hydrogen or fluoro;
R₅ is C₁₋₄alkyl;
R₆ is linear C₁₋₆alkylcarbonyl which may be substituted once or more than once by R₂₃;
R₇ is hydrogen or linear C₁₋₆alkylcarbonyl which may be substituted once or more than once by R₃₃;
each R₂₃ and R₃₃ independently is C₁₋₄alkoxy or C₁₋₄alkylthio.

One class of compounds of the invention relates to a compound of the formula I, wherein

R₁ is hydrogen, halogen, C₁₋₄alkyl, C₁₋₄halogenalkyl, C₃₋₄cycloalkyl or C₃₋₄halogencycloalkyl;
R₂ is a group A2;
m is 1 or 2;
n is 0, 1, 2 or 3;
R₁₁ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A2 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A2 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₃ is C₁₋₄halogenalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃, C₃₋₆cycloalkylcarbonyl which may be substituted once or more than once by R₂₄, phenylcarbonyl which may be substituted once or more than once by R₂₅, C₃₋₆cycloalkyl-C₁₋₂alkylcarbonyl which may be substituted once or more than once by R₂₆, phenyl-C₁₋₂alkylcarbonyl which may be substituted once or more than once by R₂₇; C₁₋₁₀alkoxyoarbonyl which may be substituted once or more than once by R₂₈, or C₃₋₆cycloalkoxycarbonyl which may be substituted once or more than once by R₂₉; phenyloxycarbonyl which may be substituted once or more than once by R₃₀, C₃₋₆cycloalkyl-C₁₋₂alkoxycarbonyl which may be substituted once or more than once by R₃₁, phenyl-C₁₋₂alkoxycarbonyl which may be substituted once or more than once by R₃₂;
R₇ is hydrogen, C₁₋₁₀alkylcarbonyl which may be substituted once or more than once by R₃₃, C₃₋₆cycloalkylcarbonyl which may be substituted once or more than once by R₃₄, phenylcarbonyl which may be substituted once or more than once by R₃₅, C₃₋₆cycloalkyl-C₁₋₂alkylcarbonyl which may be substituted once or more than once by R₃₆, phenyl-C₁₋₂alkylcarbonyl which may be substituted once or more than once by R₃₇; C₁₋₁₀alkoxycarbonyl which may be substituted once or more than once by R₃₈, or C₃₋₆cycloalkoxycarbonyl which may be substituted once or more than once by R₃₉; phenyloxycarbonyl which may be substituted once or more than once by R₄₀, C₃₋₆cycloalkyl-C₁₋₂alkoxycarbonyl which may be substituted once or more than once by R₄₁, phenyl-C₁₋₂alkoxycarbonyl which may be substituted once or more than once by R₄₂;
each R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁ and R₄₂ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein

R₁ is hydrogen, halogen, C₁₋₄alkyl, C₁₋₄halogenalkyl, C₃₋₄cycloalkyl or C₃₋₄halogencycloalkyl;
R₂ is a group A2;
m is 1 or 2;
n is 0, 1, 2 or 3;
R₁₁ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A2 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A2 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₃ is C₁₋₄halogenalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃ or C₁₋₁₀alkoxycarbonyl which may be substituted once or more than once by R₂₈;
R₇ is hydrogen, C₁₋₁₀alkylcarbonyl which may be substituted once or more than once by R₃₃ or C₁₋₁₀alkoxycarbonyl which may be substituted once or more than once by R₃₈;
each R₂₃, R₂₈, R₃₃ and R₃₈ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein

R₁ is hydrogen, halogen, C₁₋₄alkyl, C₁₋₄halogenalkyl, C₃₋₄cycloalkyl or C₃₋₄halogencycloalkyl;
R₂ is a group A2;
m is 1;
n is 0;
R₃ is C₁₋₄halogenalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃;
R₇ is hydrogen or C₁₋₁₀alkylcarbonyl which may be substituted once or more than once by

### R₃₃;

each R₂₃ and R₃₃ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

One class of compounds of the invention relates to a compound of the formula I, wherein

R₁ is hydrogen or halogen;
R₂ is a group A2;
m is 1;
n is 0;
R₃ is isopropyl, ethyl, trifluoromethyl, difluoromethyl or fluoromethyl;
R₄ is hydrogen or fluoro;
R₅ is C₁₋₄alkyl;
R₆ is linear C₁₋₆alkylcarbonyl which may be substituted once or more than once by R₂₃;
R₇ is hydrogen or linear C₁₋₆alkylcarbonyl which may be substituted once or more than once by R₃₃;
each R₂₃ and R₃₃ independently is C₁₋₄alkoxy or C₁₋₄alkylthio.

One class of compounds of the invention relates to a compound of the formula I, wherein

R₁ is hydrogen or halogen;
R₂ is a group A2;
m is 1;
n is 0;
R₃ is trifluoromethyl, difluoromethyl or fluoromethyl;
R₄ is hydrogen or fluoro;
R₅ is C₁₋₄alkyl;
R₆ is linear C₁₋₆alkylcarbonyl which may be substituted once or more than once by R₂₃;
R₇ is hydrogen or linear C₁₋₆alkylcarbonyl which may be substituted once or more than once by R₃₃;
each R₂₃ and R₃₃ independently is C₁₋₄alkoxy or C₁₋₄alkylthio.

In one embodiment, the invention provides a compound selected from
N-[6-(2-Methyl-2H-pyrazol-3-yl)-2,4-dioxo-7-trifluoromethyl-1,4-dihydro-2H-quinazolin-3-yl]-N-pentanoyl-methanesulfonamide;
N-Isobutyryl-N-[6-(2-methyl-2H-pyrazol-3-yl)-2,4-dioxo-7-trifluoromethyl-1,4-dihydro-2H-quinazolin-3-yl]-methanesulfonamide;
N-Butyryl-N-[6-(2-methyl-2H-pyrazol-3-yl)-2,4-dioxo-7-trifluoromethyl-1,4-dihydro-2H-quinazolin-3-yl]-methanesulfonamide;
N-Hexanoyl-N-[6-(2-methyl-2H-pyrazol-3-yl)-2,4-dioxo-7-trifluoromethyl-1,4-dihydro-2H-quinazolin-3-yl]-methanesulfonamide;
Methanesulfonyl-[6-(2-methyl-2H-pyrazol-3-yl)-2,4-dioxo-7-trifluoromethyl-1,4-dihydro-2H-quinazolin-3-yl]-carbamic acid methyl ester;
Methanesulfonyl-[6-(2-methyl-2H-pyrazol-3-yl)-2,4-dioxo-7-trifluoromethyl-1,4-dihydro-2H-quinazolin-3-yl]-carbamic acid isobutyl ester;
Methanesulfonyl-[6-(2-methyl-2H-pyrazol-3-yl)-2,4-dioxo-7-trifluoromethyl-1,4-dihydro-2H-quinazolin-3-yl]-carbamic acid 2-methoxy-ethyl ester;
N-Acetyl-N-[7-ethyl-6-(2-methyl-2H-pyrazol-3-yl)-2,4-dioxo-1,4-dihydro-2H-quinazolin-3-yl]-methanesulfonamide;
N-Butyryl-N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-N-hexanoyl-methanesulfonamide;
Methanesulfonyl-[7-Ethyl-6-(2-methyl-2H-pyrazol-3-yl)-2,4-dioxo-1,4-dihydro-2H-quinazolin-3-yl]-carbamic acid isobutyl ester;
Methanesulfonyl-[7-Ethyl-6-(2-methyl-2H-pyrazol-3-yl)-2,4-dioxo-1,4-dihydro-2H-quinazolin-3-yl]-carbamic acid 2-methoxy-ethyl ester;
Methanesulfonyl-[7-Ethyl-6-(2-methyl-2H-pyrazol-3-yl)-2,4-dioxo-1,4-dihydro-2H-quinazolin-3-yl]-carbamic acid propyl ester;
Methanesulfonyl-[7-Ethyl-6-(2-methyl-2H-pyrazol-3-yl)-2,4-dioxo-1,4-dihydro-2H-quinazolin-3-yl]-carbamic acid methyl ester;
Methanesulfonyl-[7-Ethyl-6-(2-methyl-2H-pyrazol-3-yl)-2,4-dioxo-1,4-dihydro-2H-quinazolin-3-yl]-carbamic acid ethyl ester;
Methanesulfonyl-[7-Ethyl-6-(2-methyl-2H-pyrazol-3-yl)-2,4-dioxo-1,4-dihydro-2H-quinazolin-3-yl]-carbamic acid butyl ester;
N-[7-Ethyl-6-(2-methyl-2H-pyrazol-3-yl)-2,4-dioxo-1,4-dihydro-2H-quinazolin-3-yl]-N-propionyl-methanesulfonamide;
N-[7-Ethyl-6-(2-methyl-2H-pyrazol-3-yl)-2,4-dioxo-1,4-dihydro-2H-quinazolin-3-yl]-N-pentanoyl-methanesulfonamide;
Methanesulfonyl-[7-ethyl-6-(2-methyl-2H-pyrazol-3-yl)-2,4-dioxo-1,4-dihydro-2H-quinazolin-3-yl]-carbamic acid isopropyl ester;
Methanesulfonyl-[7-ethyl-6-(2-methyl-2H-pyrazol-3-yl)-2,4-dioxo-1,4-dihydro-2H-quinazolin-3-yl]-carbamic acid pentyl ester;
N-Acetyl-N-[1-acetyl-7-ethyl-6-(2-methyl-2H-pyrazol-3-yl)-2,4-dioxo-1,4-dihydro-2H-quinazolin-3-yl]-methanesulfonamide;
N-Acetyl-N-[7-isopropyl-6-(2-methyl-2H-pyrazol-3-yl)-2,4-dioxo-1,4-dihydro-2H-quinazolin-3-yl]-methanesulfonamide;
[7-Isopropyl-6-(2-methyl-2H-pyrazol-3-yl)-2,4-dioxo-1,4-dihydro-2H-quinazolin-3-yl]-methanesulfonyl-carbamic acid 2-methoxy-ethyl ester;
[7-Isopropyl-6-(2-methyl-2H-pyrazol-3-yl)-2,4-dioxo-1,4-dihydro-2H-quinazolin-3-yl]-methanesulfonyl-carbamic acid isobutyl ester;
3-(Isobutoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid isobutyl ester;
[7-Isopropyl-6-(2-methyl-2H-pyrazol-3-yl)-2,4-dioxo-1,4-dihydro-2H-quinazolin-3-yl]-methanesulfonyl-carbamic acid methyl ester;
4-{[7-Isopropyl-6-(2-methyl-2H-pyrazol-3-yl)-2,4-dioxo-1,4-dihydro-2H-quinazolin-3-yl]-methanesulfonyl-amino}-4-oxo-butyric acid ethyl ester;
3-{[7-Isopropyl-6-(2-methyl-2H-pyrazol-3-yl)-2,4-dioxo-1,4-dihydro-2H-quinazolin-3-yl]-methanesulfonyl-amino}-3-oxo-propionic acid ethyl ester;
5-{[7-Isopropyl-6-(2-methyl-2H-pyrazol-3-yl)-2,4-dioxo-1,4-dihydro-2H-quinazolin-3-yl]-methanesulfonyl-amino}-5-oxo-pentanoic acid ethyl ester;
N-[7-Isopropyl-6-(2-methyl-2H-pyrazol-3-yl)-2,4-dioxo-1,4-dihydro-2H-quinazolin-3-yl]-N-pentanoyl-methanesulfonamide;
Acetic acid 2-{[7-isopropyl-6-(2-methyl-2H-pyrazol-3-yl)-2,4-dioxo-1,4-dihydro-2H-quinazolin-3-yl]-methanesulfonyl-amino}-1,1-dimethyl-2-oxo-ethyl ester;
Acetic acid 2-[3-[(2-acetoxy-2-methyl-propionyl)-methanesulfonyl-amino]-7-isopropyl-6-(2-methyl-2H-pyrazol-3-yl)-2,4-dioxo-3,4-dihydro-2H-quinazolin-1-yl]-1,1-dimethyl-2-oxo-ethyl ester;
N-[7-Isopropyl-6-(2-methyl-2H-pyrazol-3-yl)-2,4-dioxo-1,4-dihydro-2H-quinazolin-3-yl]-N-propionyl-methanesulfonamide;
N-Butyryl-N-[7-isopropyl-6-(2-methyl-2H-pyrazol-3-yl)-2,4-dioxo-1,4-dihydro-2H-quinazolin-3-yl]-methanesulfonamide;
N-Hexanoyl-N-[7-isopropyl-6-(2-methyl-2H-pyrazol-3-yl)-2,4-dioxo-1,4-dihydro-2H-quinazolin-3-yl]-methanesulfonamide;
N-Decanoyl-N-[7-isopropyl-6-(2-methyl-2H-pyrazol-3-yl)-2,4-dioxo-1,4-dihydro-2H-quinazolin-3-yl]-methanesulfonamide;
N-Isobutyryl-N-[7-isopropyl-6-(2-methyl-2H-pyrazol-3-yl)-2,4-dioxo-1,4-dihydro-2H-quinazolin-3-yl]-methanesulfonamide;
Acetic acid 2-{[7-isopropyl-6-(2-methyl-2H-pyrazol-3-yl)-2,4-dioxo-1,4-dihydro-2H-quinazolin-3-yl]-methanesulfonyl-amino}-2-oxo-ethyl ester;
N-[7-Isopropyl-6-(2-methyl-2H-pyrazol-3-yl)-2,4-dioxo-1,4-dihydro-2H-quinazolin-3-yl]-N-(3-methylsulfanyl-propionyl)-methanesulfonamide;
[7-Isopropyl-6-(2-methyl-2H-pyrazol-3-yl)-2,4-dioxo-1,4-dihydro-2H-quinazolin-3-yl]-methanesulfonyl-carbamic acid ethyl ester;
[7-Isopropyl-6-(2-methyl-2H-pyrazol-3-yl)-2,4-dioxo-1,4-dihydro-2H-quinazolin-3-yl]-methanesulfonyl-carbamic acid propyl ester;
[7-Isopropyl-6-(2-methyl-2H-pyrazol-3-yl)-2,4-dioxo-1,4-dihydro-2H-quinazolin-3-yl]-methanesulfonyl-carbamic acid butyl ester;
[7-Isopropyl-6-(2-methyl-2H-pyrazol-3-yl)-2,4-dioxo-1,4-dihydro-2H-quinazolin-3-yl]-methanesulfonyl-carbamic acid hexyl ester;
N-(2,2-Dimethyl-propionyl)-N-[7-isopropyl-6-(2-methyl-2H-pyrazol-3-yl)-2,4-dioxo-1,4-dihydro-2H-quinazolin-3-yl]-methanesulfonamide;
N-Acetyl-N-[1-acetyl-7-isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1-propionyl-1,4-dihydro-2H-quinazolin-3-yl]-N-propionyl-methanesulfonamide;
N-Butyryl-N-[1-butyryl-7-isopropyl-6-(2-methyl-2H-pyrazol-3-yl)-2,4-dioxo-1,4-dihydro-2H-quinazolin-3-yl]-methanesulfonamide;
[1-Acetyl-7-isopropyl-6-(2-methyl-2H-pyrazol-3-yl)-2,4-dioxo-1,4-dihydro-2H-quinazolin-3-yl]-methanesulfonyl-carbamic acid benzyl ester;
N-[7-Isopropyl-6-(2-methyl-2H-pyrazol-3-yl)-2,4-dioxo-1,4-dihydro-2H-quinazolin-3-yl]-N-{2-[2-(2-methoxy-ethoxy)-ethoxy]-acetyl}-methanesulfonamide;
N-(2-Benzyloxy-acetyl)-N-[7-isopropyl-6-(2-methyl-2H-pyrazol-3-yl)-2,4-dioxo-1,4-dihydro-2H-quinazolin-3-yl]-methanesulfonamide;
N-(4-Benzyloxy-butyryl)-N-[7-isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-N-(2-morpholin-4-yl-acetyl)-methanesulfonamide;
7-Isopropyl-3-(methoxycarbonyl-methanesulfonyl-amino)-6-(2-methyl-2H-pyrazol-3-yl)-2,4-dioxo-3,4-dihydro-2H-quinazoline-1-carboxylic acid methyl ester;
3-(Ethoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid ethyl ester;
7-Isopropyl-3-(methanesulfonyl-propoxycarbonyl-amino)-6-(2-methyl-2H-pyrazol-3-yl)-2,4-dioxo-3,4-dihydro-2H-quinazoline-1-carboxylic acid propyl ester;
3-(Butoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid butyl ester;
3-(Acetyl-methanesulfonyl-amino)-7-isopropyl-6-(2-methyl-2H-pyrazol-3-yl)-2,4-dioxo-3,4-dihydro-2H-quinazoline-1-carboxylic acid methyl ester;
N-[7-Isopropyl-6-(2-methyl-2H-pyrazol-3-yl)-2,4-dioxo-1,4-dihydro-2H-quinazolin-3-yl]-N-(4-morpholin-4-yl-butyryl)-methanesulfonamide;
N-[7-Isopropyl-6-(2-methyl-2H-pyrazol-3-yl)-2,4-dioxo-1,4-dihydro-2H-quinazolin-3-yl]-N-(3-morpholin-4-yl-propionyl)-methanesulfonamide;
4-[(6-Imidazol-1-yl-2,4-dioxo-7-trifluoromethyl-1,4-dihydro-2H-quinazolin-3-yl)-methanesulfonyl-amino]-4-oxo-butyric acid ethyl ester;
N-(6-Imidazol-1-yl-2,4-dioxo-7-trifluoromethyl-1,4-dihydro-2H-quinazolin-3-yl)-N-pentanoyl-methanesulfonamide;
N-Acetyl-N-(6-imidazol-1-yl-2,4-dioxo-7-trifluoromethyl-1,4-dihydro-2H-quinazolin-3-yl)-methanesulfonamide;
N-(6-Imidazol-1-yl-2,4-dioxo-7-trifluoromethyl-1,4-dihydro-2H-quinazolin-3-yl)-N-propionyl-methanesulfonamide;
N-(6-Imidazol-1-yl-2,4-dioxo-7-trifluoromethyl-1,4-dihydro-2H-quinazolin-3-yl)-N-isobutyryl-methanesulfonamide;
N-(6-Imidazol-1-yl-2,4-dioxo-7-trifluoromethyl-1,4-dihydro-2H-quinazolin-3-yl)-N-(3-methyl-butyryl)-methanesulfonamide;
N-Cyclopentanecarbonyl-N-(6-imidazol-1-yl-2,4-dioxo-7-trifluoromethyl-1,4-dihydro-2H-quinazolin-3-yl)-methanesulfonamide;
N-Hexanoyl-N-(6-imidazol-1-yl-2,4-dioxo-7-trifluoromethyl-1,4-dihydro-2H-quinazolin-3-yl)-methanesulfonamide;
N-Butyryl-N-(6-imidazol-1-yl-2,4-dioxo-7-trifluoromethyl-1,4-dihydro-2H-quinazolin-3-yl)-methanesulfonamide;
N-Acetyl-N-[6-(2-isopropyl-2H-pyrazol-3-yl)-2,4-dioxo-7-trifluoromethyl-1,4-dihydro-2H-quinazolin-3-yl]-methanesulfonamide;
N-[6-(2-isopropyl-2H-pyrazol-3-yl)-2,4-dioxo-7-trifluoromethyl-1,4-dihydro-2H-quinazolin-3-yl]-N-propionyl-methanesulfonamide;
N-Butyryl-N-[6-(2-isopropyl-2H-pyrazol-3-yl)-2,4-dioxo-7-trifluoromethyl-1,4-dihydro-2H-quinazolin-3-yl]-methanesulfonamide;
N-Hexanoyl-N-[6-(2-isopropyl-2H-pyrazol-3-yl)-2,4-dioxo-7-trifluoromethyl-1,4-dihydro-2H-quinazolin-3-yl]-methanesulfonamide;
N-Isobutyryl-N-[6-(2-isopropyl-2H-pyrazol-3-yl)-2,4-dioxo-7-trifluoromethyl-1,4-dihydro-2H-quinazolin-3-yl]-methanesulfonamide;
[6-(2-Isopropyl-2H-pyrazol-3-yl)-2,4-dioxo-7-trifluoromethyl-1,4-dihydro-2H-quinazolin-3-yl]-methanesulfonyl-carbamic acid ethyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid propyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid butyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid isobutyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid 2-methoxy-ethyl ester;
5-{[6-(2-Isopropyl-2H-pyrazol-3-yl)-2,4-dioxo-7-trifluoromethyl-1,4-dihydro-2H-quinazolin-3-yl]-methanesulfonyl-amino}-5-oxo-pentanoic acid ethyl ester;
3-{[6-(2-Isopropyl-2H-pyrazol-3-yl)-2,4-dioxo-7-trifluoromethyl-1,4-dihydro-2H-quinazolin-3-yl]-methanesulfonyl-amino}-3-oxo-propionic acid ethyl ester;
N-[6-(2-Isopropyl-2H-pyrazol-3-yl)-2,4-dioxo-7-trifluoromethyl-1,4-dihydro-2H-quinazolin-3-yl]-N-(3-methylsulfanyl-propionyl)-methanesulfonamide;
N-Acetyl-N-[7-difluoromethyl-6-(2-isopropyl-2H-pyrazol-3-yl)-2,4-dioxo-1,4-dihydro-2H-quinazolin-3-yl]-methanesulfonamide;
N-Butyryl-N-[7-difluoromethyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-N-pentanoyl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid propyl ester;
N-[7-Difluoromethyl-6-(2-isopropyl-2H-pyrazol-3-yl)-2,4-dioxo-1,4-dihydro-2H-quinazolin-3-yl]-N-propionyl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid ethyl ester;
[7-Difluoromethyl-6-(2-isopropyl-2H-pyrazol-3-yl)-2,4-dioxo-1,4-dihydro-2H-quinazolin-3-yl]-methanesulfonyl-carbamic acid 2-methoxy-ethyl ester;
Methanesulfonyl-[6-(2-methyl-imidazol-1-yl)-2,4-dioxo-7-trifluoromethyl-1,4-dihydro-2H-quinazolin-3-yl]-carbamic acid ethyl ester;
N-Butyryl-N-[6-(2-methyl-imidazol-1-yl)-2,4-dioxo-7-trifluoromethyl-1,4-dihydro-2H-quinazolin-3-yl]-methanesulfonamide;
Methanesulfonyl-[6-(2-methyl-imidazol-1-yl)-2,4-dioxo-7-trifluoromethyl-1,4-dihydro-2H-quinazolin-3-yl]-carbamic acid propyl ester;
Methanesulfonyl-[6-(2-methyl-imidazol-1-yl)-2,4-dioxo-7-trifluoromethyl-1,4-dihydro-2H-quinazolin-3-yl]-carbamic acid isobutyl ester;
N-(2,4-Dioxo-6-pyrrol-1-yl-7-trifluoromethyl-1,4-dihydro-2H-quinazolin-3-yl)-N-propionyl-methanesulfonamide;
N-Butyryl-N-(2,4-dioxo-6-pyrrol-1-yl-7-trifluoromethyl-1,4-dihydro-2H-quinazolin-3-yl)-methanesulfonamide;
Acetic acid 2-[(2,4-dioxo-6-pyrrol-1-yl-7-trifluoromethyl-1,4-dihydro-2H-quinazolin-3-yl)-methanesulfonyl-amino]-2-oxo-ethyl ester;
(2,4-Dioxo-6-pyrrol-1-yl-7-trifluoromethyl-1,4-dihydro-2H-quinazolin-3-yl)-methanesulfonyl-carbamic acid methyl ester;
(2,4-Dioxo-6-pyrrol-1-yl-7-trifluoromethyl-1,4-dihydro-2H-quinazolin-3-yl)-methanesulfonyl-carbamic acid ethyl ester;
N-(2,4-Dioxo-6-[1,2,4]triazol-4-yl-7-trifluoromethyl-1,4-dihydro-2H-quinazolin-3-yl)-N-(3-methyl-butyryl)-methanesulfonamide;
N-(2,4-Dioxo-6-[1,2,4]triazol-4-yl-7-trifluoromethyl-1,4-dihydro-2H-quinazolin-3-yl)-N-hexanoyl-methanesulfonamide;
N-(2,4-Dioxo-6-[1,2,4]triazol-4-yl-7-trifluoromethyl-1,4-dihydro-2H-quinazolin-3-yl)-N-pentanoyl-methanesulfonamide;
N-Butyryl-N-(2,4-dioxo-6-[1,2,4]triazol-4-yl-7-trifluoromethyl-1,4-dihydro-2H-quinazolin-3-yl)-methanesulfonamide;
N-Acetyl-N-(2,4-dioxo-6-tetrahydro-furan-2-yl-7-trifluoromethyl-1,4-dihydro-2H-quinazolin-3-yl)-methanesulfonamide;
N-(2,4-dioxo-6-tetrahydro-furan-2-yl-7-trifluoromethyl-1,4-dihydro-2H-quinazolin-3-yl)-N-propionyl-methanesulfonamide;
N-Butyryl-N-(2,4-dioxo-6-tetrahydro-furan-2-yl-7-trifluoromethyl-1,4-dihydro-2H-quinazolin-3-yl)-methanesulfonamide;
Methanesulfonyl-N-(2,4-dioxo-6-tetrahydro-furan-2-yl-7-trifluoromethyl-1,4-dihydro-2H-quinazolin-3-yl)-carbamic acid methyl ester;
Methanesulfonyl-N-(2,4-dioxo-6-tetrahydro-furan-2-yl-7-trifluoromethyl-1,4-dihydro-2H-quinazolin-3-yl)-carbamic acid ethyl ester;
Methanesulfonyl-N-(2,4-dioxo-6-tetrahydro-furan-2-yl-7-trifluoromethyl-1,4-dihydro-2H-quinazolin-3-yl)-carbamic acid butyl ester;
Methanesulfonyl-N-(2,4-dioxo-6-tetrahydro-furan-2-yl-7-trifluoromethyl-1,4-dihydro-2H-quinazolin-3-yl)-carbamic acid 2-methoxy-ethyl ester;
Methanesulfonyl-N-(2,4-dioxo-6-tetrahydro-furan-2-yl-7-trifluoromethyl-1,4-dihydro-2H-quinazolin-3-yl)-carbamic acid isobutyl ester;
N-(2,4-dioxo-6-tetrahydro-furan-2-yl-7-trifluoromethyl-1,4-dihydro-2H-quinazolin-3-yl)-N-hexanoyl-methanesulfonamide;
4-[(2,4-Dioxo-6-tetrahydro-furan-2-yl-7-trifluoromethyl-1,4-dihydro-2H-quinazolin-3-yl)-methanesulfonyl-amino]-4-oxo-butyric acid ethyl ester;
N-(2,4-Dioxo-6-tetrahydro-furan-2-yl-7-trifluoromethyl-1,4-dihydro-2H-quinazolin-3-yl)-N-pentanoyl-methanesulfonamide;
Methanesulfonyl-[6-(1-methoxy-propyl)-2,4-dioxo-7-trifluoromethyl-1,4-dihydro-2H-quinazolin-3-yl]-carbamic acid 2-methoxy-ethyl ester;
N-[6-(1-Methoxy-propyl)-2,4-dioxo-7-trifluoromethyl-1,4-dihydro-2H-quinazolin-3-yl]-N-propionyl-methanesulfonamide;
Methanesulfonyl-[6-(1-methoxy-propyl)-2,4-dioxo-7-trifluoromethyl-1,4-dihydro-2H-quinazolin-3-yl]-carbamic acid ethyl ester;
Methanesulfonyl-[6-(1-methoxy-propyl)-2,4-dioxo-7-trifluoromethyl-1,4-dihydro-2H-quinazolin-3-yl]-carbamic acid propyl ester;
N-Hexanoyl-N-[6-(1-Methoxy-propyl)-2,4-dioxo-7-trifluoromethyl-1,4-dihydro-2H-quinazolin-3-yl]-methanesulfonamide;
N-Isobutyryl-N-[6-(1-methoxy-propyl)-2,4-dioxo-7-trifluoromethyl-1,4-dihydro-2H-quinazolin-3-yl]-methanesulfonamide;
N-Butyryl-N-[6-(1-methoxy-propyl)-2,4-dioxo-7-trifluoromethyl-1,4-dihydro-2H-quinazolin-3-yl]-methanesulfonamide;
N-Acetyl-N-[6-(1-methoxy-propyl)-2,4-dioxo-7-trifluoromethyl-1,4-dihydro-2H-quinazolin-3-yl]-methanesulfonamide;
Methanesulfonyl-[6-(1-methoxy-propyl)-2,4-dioxo-7-trifluoromethyl-1,4-dihydro-2H-quinazolin-3-yl]-carbamic acid methyl ester;
Methanesulfonyl-[6-(1-methoxy-propyl)-2,4-dioxo-7-trifluoromethyl-1,4-dihydro-2H-quinazolin-3-yl]-carbamic acid isobutyl ester;
Methanesulfonyl-[6-(1-methoxy-propyl)-2,4-dioxo-7-trifluoromethyl-1,4-dihydro-2H-quinazolin-3-yl]-carbamic acid butyl ester;
Methanesulfonyl-[6-(1-methoxy-propyl)-2,4-dioxo-7-trifluoromethyl-1,4-dihydro-2H-quinazolin-3-yl]-carbamic acid pentyl ester;
Methanesulfonyl-[6-(1-methoxy-propyl)-2,4-dioxo-7-trifluoromethyl-1,4-dihydro-2H-quinazolin-3-yl]-carbamic acid hexyl ester;
Methanesulfonyl-[6-(1-methoxy-propyl)-2,4-dioxo-7-trifluoromethyl-1,4-dihydro-2H-quinazolin-3-yl]-carbamic acid isopropyl ester;
N-[6-(1-methoxy-propyl)-2,4-dioxo-7-trifluoromethyl-1,4-dihydro-2H-quinazolin-3-yl]-N-pentanoyl-methanesulfonamide;
N-Hexanoyl-N-[6-(1-hydroxy-propyl)-2,4-dioxo-7-trifluoromethyl-1,4-dihydro-2H-quinazolin-3-yl]-methanesulfonamide;
Methanesulfonyl-[6-(1-hydroxy-propyl)-2,4-dioxo-7-trifluoromethyl-1,4-dihydro-2H-quinazolin-3-yl]-carbamic acid 2-methoxy-ethyl ester;
N-Butyryl-N-[6-(1-hydroxy-propyl)-2,4-dioxo-7-trifluoromethyl-1,4-dihydro-2H-quinazolin-3-yl]-methanesulfonamide;
N-[6-(1-hydroxy-propyl)-2,4-dioxo-7-trifluoromethyl-1,4-dihydro-2H-quinazolin-3-yl]-N-isobutyryl-methanesulfonamide;
N-[6-(1-hydroxy-propyl)-2,4-dioxo-7-trifluoromethyl-1,4-dihydro-2H-quinazolin-3-yl]-N-pentanoyl-methanesulfonamide;
Methanesulfonyl-[7-Ethyl-6-(2-ethyl-2H-pyrazol-3-yl)-2,4-dioxo-1,4-dihydro-2H-quinazolin-3-yl]carbamic acid isobutyl ester;
N-[7-Ethyl-6-(2-ethyl-2H-pyrazol-3-yl)-2,4-dioxo-1,4-dihydro-2H-quinazolin-3-yl]-N-hexanoyl-methanesulfonamide;
N-[7-Ethyl-6-(2-ethyl-2H-pyrazol-3-yl)-2,4-dioxo-1,4-dihydro-2H-quinazolin-3-yl]-N-propionyl-methanesulfonamide;
Acetic acid 2-{[7-difluoromethyl-6-(2-ethyl-2H-pyrazol-3-yl)-2,4-dioxo-1,4-dihydro-2H-quinazolin-3-yl]-methanesulfonyl-amino}-2-oxo-ethyl ester;
[6-(2-Ethyl-2H-pyrazol-3-yl)-2,4-dioxo-7-trifluoromethyl-1,4-dihydro-2H-quinazolin-3-yl]-methanesulfonyl-carbamic acid methyl ester; N-[6-(2-Ethyl-2H-pyrazol-3-yl)-2,4-dioxo-7-trifluoromethyl-1,4-dihydro-2H-quinazolin-3-yl]-N-propionyl-methanesulfonamide;
[6-(2-Ethyl-2H-pyrazol-3-yl)-2,4-dioxo-7-trifluoromethyl-1,4-dihydro-2H-quinazolin-3-yl]-methanesulfonyl-carbamic acid butyl ester; and
N-(2,2-Dimethyl-propionyl)-N-[6-(2-ethyl-2H-pyrazol-3-yl)-2,4-dioxo-7-trifluoromethyl-1,4-dihydro-2H-quinazolin-3-yl]-methanesulfonamide.

In a further aspect, the invention also provides a process for the production of compounds of the invention. Compounds of the invention are obtainable according to the following processes as described in scheme 1 or scheme 2:

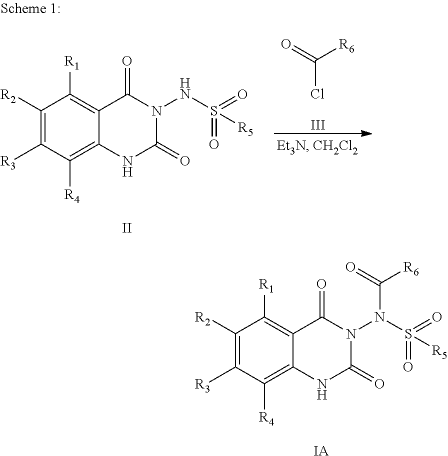

According to scheme 1, compounds of the formula (IA), in which R₁, R₂, R₃, R₄, R₅ and R₆ are as defined under formula I, may be obtained by reacting a compound of formula (II), in which R₁, R₂, R₃, R₄ and R₅ are as defined under formula I, with a compound of formula (III), in which R₆ is as defined under formula I, in the presence of a base, such as triethylamine.

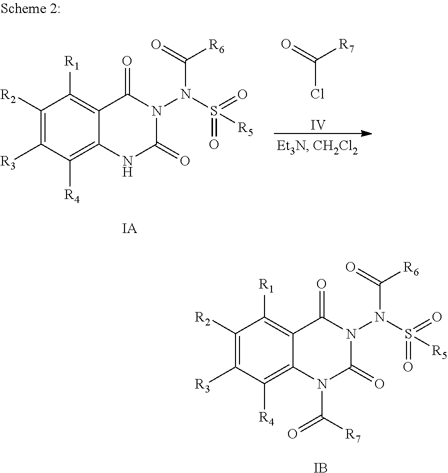

According to scheme 1, compounds of the formula (IB), in which R₁, R₂, R₃, R₄, R₅, R₆ and R₇ are as defined under formula I, may be obtained by reacting a compound of formula (IA), in which R₁, R₂, R₃, R₄, R₅ and R₆ are as defined under formula I, with a compound of formula (IV), in which R7₆ is as defined under formula I, in the presence of a base, such as triethylamine.

The processes of scheme 1 and/or scheme 2 are further described via Methods A1, A2, B1, B2.1, B2.2, C1, C2, D, E1, E2 and/or F below.

### Method A1:

The starting sulfonamide (a compound of formula II, Scheme 1) (1.0 equiv.) is suspended in triethylamine (1.5 equiv.) and dry dichloromethane at room temperature (22° C.). Sequentially the corresponding acid chloride (1.1 equiv.) is added and the reaction mixture is stirred at room temperature. After 1.5 hours additional triethylamine (0.75 equiv.) and acid chloride (0.55 equiv.) is added and the reaction mixture is stirred for another 1.5 hours. Subsequently the crude reaction mixture is poured onto a flash column and subjected to silica gel flash chromatography (ISCO CombiFlash) using the appropriate eluent (typically dichloromethane/methanol; 100/0 to 90/10).

### Method A2:

The starting sulfonamide (a compound of formula II, Scheme 1) (1.0 equiv.) is suspended in triethylamine (1.5 equiv.) and dry acetonitrile at room temperature (22° C.). Sequentially the corresponding acid chloride (1.1 equiv.) is added and the reaction mixture is stirred at room temperature. After 1.5 hours additional triethylamine (0.75 equiv.) and acid chloride (0.55 equiv.) is added and the reaction mixture is stirred for another 1.5 hours. Subsequently the crude reaction mixture is poured onto a preparative HPLC column and subjected to RP18 chromatography (Gilson prep. HPLC) using the appropriate eluent (typically 0.1% TFA/acetonitril+0.1% TFA, gradient from 95:5 to 5:95 in 20 min).

### Method B1:

A 0.1 molar solution of the corresponding sulfonamide in dimethylformamide is treated with 1.2 eq of NaH. The reaction is stirred for 30 min at 22° C., when the alkylcarboxylic acid chloride (1.2 eq.) is added. The reaction is stirred for 18 h at room temperature and then poured on ice-cooled water and diluted with ethyl acetate. The organic layers are separated and washed with brine, dried over MgSO4, filtered, and concentrated in vacuo. The residue is purified by preparatory HPLC (reverse phase) to yield the product.

### Method B2.1:

A 0.1 molar solution of the corresponding sulfonamide in tetrahydrofurane is treated with 2.2 eq of NaH. The reaction is stirred for 30 min at 22° C., when the acid chloride (3.5 eq.) is added. The reaction is stirred for 1 h at room temperature and then poured on ice-cooled aq. citric acid solution (5%) and diluted with ethyl acetate. The organic layers are separated and washed with brine, dried over MgSO4, filtered, and concentrated in vacuo. The residue is purified by silica gel flash chromatography (ISCO CombiFlash) using the appropriate eluent gradient (typically dichloromethane/methanol; methanol 0% to 10%).

### Method B2.2:

A 0.1 molar solution of the corresponding sulfonamide in tetrahydrofurane is treated with 1.1 eq of NaH. The reaction is stirred for 30 min at 22° C., when the acid chloride (2 eq.) is added. The reaction is stirred for 1 h at room temperature and then poured on ice-cooled aq. citric acid solution (5%) and diluted with ethyl acetate. The organic layers are separated and washed with brine, dried over MgSO4, filtered, and concentrated in vacuo. The residue is purified by silica gel flash chromatography (ISCO CombiFlash) using the appropriate eluent gradient (typically dichloromethane/methanol; methanol 0% to 10%).

### Method C1:

The starting sulfonamide (a compound of formula II, Scheme 1) (1.0 equiv.) is dissolved in pyridine (19 equiv.) at room temperature (22° C.). Sequentially the corresponding acid chloride (1.5 equiv.) is added and the reaction mixture is stirred at room temperature for 1 hour. Subsequently the solvent is evaporated and the crude product is purified by silica gel flash chromatography (ISCO CombiFlash) using the appropriate eluent gradient (typically hexan/ethyl acetate; ethyl acetate 0% to 100%).

### Method C2:

The starting sulfonamide (a compound of formula II, Scheme 1) (1.0 equiv.) is dissolved in dry dichloromethane and pyridine (1.25 equiv.) at room temperature (22° C.). Sequentially the corresponding acid chloride (1.1 equiv.) is added and the reaction mixture is stirred at room temperature for 1 hour. Subsequently the solvent is evaporated and the crude product is purified by silica gel flash chromatography (ISCO CombiFlash) using the appropriate eluent gradient (typically hexan/ethyl acetate; ethyl acetate 0% to 100%).

### Method D:

The starting sulfonamide (a compound of formula II, Scheme 1) (1.0 equiv.) is suspended in triethylamine (2.2 equiv.) and dry dichloromethane at room temperature (22° C.). Sequentially the corresponding acid chloride (4.0 equiv.) is added and the reaction mixture is stirred at room temperature. Subsequently the crude reaction mixture is poured onto a flash column after 1.5 hour and subjected to silica gel flash chromatography (ISCO CombiFlash) using the appropriate eluent (typically dichloromethane/methanol; 100/0 to 90/10).

### Method E1:

The starting sulfonamide (a compound of formula II, Scheme 1) (1.0 equiv.) is suspended in diisopropyl-ethylamine (1.2 equiv.) and dry dichloromethane at room temperature (22° C.). Sequentially the corresponding acid chloride (1.1 equiv.) is added and the reaction mixture is stirred at room temperature. After 1.5 hours the crude reaction mixture is poured on water and extracted three times with dichloromethane. The organic layer was dried over sodiumsulfate and evaporated. The crude product was purified by flash chromatography (ISCO CombiFlash) using the appropriate eluent (typically dichloromethane/methanol; 100/0 to 90/10).

### Method E2:

The starting sulfonamide (a compound of formula II, Scheme 1) (1.0 equiv.) is suspended in diisopropyl-ethylamine (2.2 equiv.) and dry dichloromethane at room temperature (22° C.). Sequentially the corresponding acid chloride (1.1 equiv.) is added and the reaction mixture is stirred at room temperature. After 1.5 hours the crude reaction mixture is poured on water and extracted three times with dichloromethane. The organic layer was dried over sodiumsulfate and evaporated. The crude product was purified by flash chromatography (ISCO CombiFlash) using the appropriate eluent (typically dichloromethane/methanol; 100/0 to 90/10).

### Method F:

The starting sulfonamide (a compound of formula II, Scheme 1) (1.0 equiv.) is suspended in dry dichloromethane (0.1 molar) at room temperature (22° C.). Sequentially the corresponding acid (1.1 equiv.), EDC (1.25 equiv.), HOAt (1.5 equiv.) and triethylamine (2.5 equiv.) is added and the reaction mixture is stirred at room temperature. After 2 hours the crude reaction mixture was purified by flash chromatography (ISCO CombiFlash) using the appropriate eluent (typically dichloromethane/methanol; 100/0 to 90/10).

Further compounds of formula (I) may be obtainable from compounds of formula IA or from compounds of formula IB prepared as described according to scheme 1 or scheme 2-by reduction, oxidation and/or other functionalization of resulting compounds and/or by cleavage of any protecting group(s) optionally present, and of recovering the so obtainable compound of the formula (I).

The reactions can be effected according to conventional methods, for example as described in the Examples.

The work-up of the reaction mixtures and the purification of the compounds thus obtainable may be carried out in accordance with known procedures.

Acid addition salts may be produced from the free bases in known manner, and vice-versa.

Compounds of the formula I can also be prepared by further conventional processes, e.g. as described in the Examples, which processes are further aspects of the invention.

The starting materials of scheme 1 and/or scheme 2 are known (e.g. compounds of formula (II) from WO2006108591 and WO2006010591, respectively) or may be prepared according to conventional procedures starting from known compounds, for example as described in the Examples.

In another aspect, the invention provides a pharmaceutical composition comprising a therapeutically effective amount of a compound of the invention and one or more pharmaceutically acceptable carriers.

The pharmaceutical composition can be formulated for particular routes of administration such as oral administration, parenteral administration, and rectal administration, etc. In addition, the pharmaceutical compositions of the invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers etc.

Typically, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The invention further provides anhydrous pharmaceutical compositions and dosage forms comprising the compounds of the invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the invention as an active ingredient will decompose. Such agents, which are referred to herein as “stabilizers,” include, but are not limited to, antioxidants such as ascorbic acid, pH buffers, or salt buffers, etc.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the invention refers to an amount of the compound of the invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the invention that, when administered to a subject, is effective to (1) at least partially alleviating, inhibiting, preventing and/or ameliorating a condition, or a disorder or a disease (i) mediated by AMPA and/or kainate receptors, or (ii) associated with AMPA and/or kainate receptor activity, or (iii) characterized by abnormal activity of AMPA and/or kainate receptors; or (2) reducing or inhibiting the activity of AMPA and/or kainate receptors; or (3) reducing or inhibiting the expression of AMPA and/or kainate receptors. In another non-limiting embodiment, the term “a therapeutically effective amount” refers to the amount of the compound of the invention that, when administered to a cell, or a tissue, or a non-cellular biological material, or a medium, is effective to at least partially reducing or inhibiting the activity of AMPA and/or kainate receptors; or at least partially reducing or inhibiting the expression of AMPA and/or kainate receptors.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

The compounds of the invention in free form or in pharmaceutically acceptable salt form, exhibit valuable pharmacological properties, e.g. an AMPA or a dual AMPA/kainate receptor antagonism is effected when administered to patients, e.g. as indicated in in-vitro and/or in-vivo tests as provided in the sections below. Therefore, the compounds of the invention in free form or in pharmaceutically acceptable salt form are indicated for therapy.

The compounds of the invention are especially effective as pharmaceuticals in the treatment of epilepsy, esp. in partial seizures (simple, complex and partial evolving to secondarily generalized seizures) and generalized seizures [absence (typical and atypical), myoclonic, clonic, tonic, tonic-clonic and atonic]. The term “epilepsy” includes epilepsy in patients having an abnormal serum level of anti-GluR3 auto-antibodies.

Furthermore, the compounds of the invention are useful as pharmaceuticals in the treatment of any pathology, disorder or clinical condition involving altered AMPA and/or kainate receptor function or AMPA and/or kainate receptor mediated neuronal damage, e.g. neurodegenerative disorders, such as multiple sclerosis, amyotrophic lateral sclerosis, neuronal ceroid lipofuscinosis (NCL; e.g. Batten disease, Infantile NCL, Late infantile NCL, Adult NCL, Finnish Late Infantile NCL, Portuguese Late Infantile NCL, Turkish Late Infantile NCL or Progressive Epilepsy with Mental Retardation), Rasmussen's encephalitis, Parkinson's Disease, Huntington's Disease or Alzheimers Disease, schizophrenia, esp. chronic schizophrenia, psychosis, anxiety, depression, bipolar mood disorders, sleep disorders, cognitive disorders, emesis, tinnitus, muscle spasticity, muscle rigidity, pain, neuropathic pain, migraine, migraine prophylaxis, tension headache, cluster headache, complex regional pain syndrome, myopia, tumor growth, drug-withdrawal symptoms, ischemic and hypoxic conditions such as stroke, subarachnoid haemorrhage, perinatal hypoxia, brain and spinal cord trauma, head injury, high intracranial pressure, and any surgical procedure potentially associated with hypoxia of the central nervous system, and conditions produced by the actions of environmental, exogenous neurotoxins, including those produced by infections as well as those produced by metabolic changes and hepatic encephalopathy associated with liver failure.

Compounds of the invention may be especially useful in the treatment of an indication selected from: epilepsy, migraine and tinnitus.

Thus, as a further embodiment, the invention provides a compound of the invention in free form or in pharmaceutically acceptable salt form for use as a medicament.

Thus, as a further embodiment, the invention provides the use of a compound of the invention in free form or in pharmaceutically acceptable salt form as a medicament.

As a further embodiment, the invention provides the use of a compound of the invention in free form or in pharmaceutically acceptable salt form in therapy.

In a further embodiment, the therapy is selected from a disease which is ameliorated by antagonism of AMPA and/or kainate receptors. In another embodiment, the disease is selected from the afore-mentioned list, suitably epilepsy, migraine and tinnitus.

In another embodiment, the invention provides a method of treating a disease which is ameliorated by antagonism of AMPA and/or kainate receptors comprising administration of a therapeutically acceptable amount of a compound of the invention in free form or in pharmaceutically acceptable salt form. In a further embodiment, the disease is selected from the afore-mentioned list, suitably epilepsy, migraine and tinnitus.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The pharmaceutical composition of the invention or—as described below—the combination of the invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The activity of a compound of the invention can be assessed by in vitro and/or in vivo methods described herein.

The compound of the invention may be administered either simultaneously with, or before or after, at least one other therapeutic agent. The compound of the invention may be administered separately, by the same or different route of administration, or together in the same pharmaceutical composition. The compounds of the invention can be combined, e.g. in the case of epilepsy, with other anti-epileptic drugs like barbiturates and derivatives thereof, benzodiazepines, carboxamides, hydantoins, succinimides, valproic acid and other fatty acid derivates, Lamotrigine, Levetiracetam and derivatives thereof, Topiramate, Pregabalin, Gabapentin, Zonisamide, Sultiam, Felbamate, Lacosamide, Retigabine and other AMPA-receptor and AMPA/kainate receptor antagonists. The compounds of the invention can also be combined with neuroleptic drugs selected from the list consisting of atypical antipsychotic drugs such as clozapine, olanzapine, risperidone and typical antipsychotic drugs such as haloperidol.

Thus, the invention further provides a combination comprising a therapeutically effective amount of a compound of the invention and one or more therapeutically active agents, in one embodiment, said combination is a combined preparation.

Thus, in further aspects, the present invention relates to
Combinations comprising a compound of the invention and Anti-epileptic Drugs, suitable for the treatment of Neurological Disorders

The present invention also relates to combinations suitable for the treatment of neurological disorders, in particular epilepsy, e.g a combination which comprises at least two anti-epileptic drugs, one being a compound of the invention and the other being selected from the list consisting of barbiturates and derivatives thereof, benzodiazepines, carboxamides, hydantoins, succinimides, valproic acid and other fatty acid derivates, AMPA-receptor antagonists and other anti-epileptic drugs.
Combinations comprising a compound of the invention for affective and attention disorders

The present invention also relates to combinations suitable for the treatment of neurological/psychiatric disorders, in particular affective and attention disorders, e.g a combination which comprises at least one compound of the invention and at least one compound selected from the group consisting of lithium, valproic acid sodium salt, conventional antipsychotics, atypical antipsychotics, lamotrigine, methylphenidate, antidepressants and antiepileptics.
Combinations comprising a compound of the invention suitable for the treatment of neurological/psychiatric disorders

The present invention also relates to combinations suitable for the treatment of neurological/psychiatric disorders, in particular anxiety disorders or other psychiatric disorders with underlying anxiety symptomatologies, e.g a combination which comprises at least one compound of the invention and at least one compound selected from the group consisting of benzodiazepines, selective serotonin reuptake inhibitors (SSRIs), selective serotonin and norepinephrine reuptake inhibitors (SNRIs), buspirone and pregabalin.
Combinations comprising a compound of the invention suitable for the treatment of ocular disorders, in particular myopia

The present invention also relates to combinations suitable for the treatment of ocular disorders, in particular myopia, e.g a combination which comprises at least one compound of the invention and at least one compound selected from the group consisting of pirenzepine, telenzepine, ortho-methoxy-sila-hexocyclium, gamma-amino butyric acid (GABA) and GABA-receptor agonists.
Combinations comprising a compound of the invention suitable for the treatment of pain, especially neuropathic pain

The present invention also relates to combinations suitable for the treatment of pain, especially neuropathic pain, e.g. a combination which comprises at least one compound of the invention and at least one combination partner selected from the group consisting of cyclooxygenase inhibitors, vanilloid receptor antagonists, opioids, tricyclic antidepressants, cathepsin S inhibitors, cannabinoid receptor antagonists and GABA_{B} receptor agonists.
Combinations comprising a compound of the invention suitable for the treatment of migraine

The present invention also relates to combinations suitable for the treatment of migraine, e.g. a combination which comprises at least one compound of the invention and at least one combination partner selected from the group consisting of 5-HT_{1B/1D} receptor agonists (e.g. “triptans”), antiemetics, ergot derivatives and analgesics, e.g. NSAIDs. Examples of 5-HT_{1B/1D} receptor agonists are tryptamine-based drugs (also known as “triptans”), e.g. almotriptan (“Axert”™, Almogran”™), eletriptan (“Relpax”™), frovatriptan (“Frova”™, “Migard”™), naratriptan (“Amerge”™, “Naramig”™), rizatriptan (“Maxalt”™), sumatriptan (“Imitrex”™, “Imigran”™) or zolmitriptan (“Zomig”™); ergotamine; or dihydroergotamine.
Combinations comprising a compound of the invention suitable for the treatment of psychiatric/neurological disorders, in particular schizophrenia.

The present invention also relates to combinations suitable for the treatment of psychiatric/neurological disorders, in particular schizophrenia. E.g. a combination, such as a combined preparation or pharmaceutical composition, which comprises at least one compound of the invention and at least one compound selected from the group consisting of conventional antipsychotics or atypical antipsychotics including metabotropic glutamate receptor active compounds.
Combinations comprising a compound of the invention suitable for the treatment of Parkinson's disease.

The present invention also relates to combinations suitable for the treatment of Parkinson's disease, e.g. a combination which comprises at least one compound of the invention and at least one combination partner selected from the group consisting of dopaminergic agonists (e.g. levodopa), anticholinergic drugs, or antihistamines.
Combinations comprising a compound of the invention suitable for the use in anesthesia.

The present invention also relates to combinations suitable for the use in anesthesia e.g. a combination which comprises at least one compound of the invention and at least one combination partner selected from the group consisting of inhalation anesthetics (e.g. halothane, isoflurane), other injectable anesthetics (e.g. propofol), injectable analgesics (e.g. opioids) and injectable sedatives (e.g. benzodiazepines).

The term “a combined preparation”, as used herein defines especially a “kit of parts” in the sense that the first and second active ingredient as defined above can be dosed independently or by use of different fixed combinations with distinguished amounts of the ingredients, i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient 1 to the active ingredient 2 to be administered in the combined preparation can be varied, e.g., in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the first and second active ingredient, in particular a synergism, e.g. a more than additive effect, additional advantageous effects, less side effects, a combined therapeutical effect in a non-effective dosage of one or both of the first and second active ingredient, and especially a strong synergism between the first and second active ingredient.

It will be understood that in the discussion of methods, references to the active ingredients are meant to also include the pharmaceutically acceptable salts. If these active ingredients have, for example, at least one basic center, they can form acid addition salts. Corres-ponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

In particular, a therapeutically effective amount of each of the active ingredients of a combination may be administered simultaneously or sequentially and in any order, and the components may be administered separately or as a fixed combination. For example, the method of treatment of diseases according to the invention may comprise (i) administration of the first active ingredient in free or pharmaceutically acceptable salt form and (ii) administration of the second active ingredient in free or pharmaceutically acceptable salt form, simultaneously or sequentially in any order, in jointly therapeutically effective amounts, preferably in synergistically effective amounts, e.g. in daily dosages corresponding to the amounts described herein. The individual active ingredients of the combination can be administered separately at different times during the course of therapy or concurrently in divided or single combination forms. Furthermore, the term administering also encompasses the use of a prodrug of an active ingredient that convert in vivo to the active ingredient. The instant invention is therefore to be understood as embracing all such regimes of simultaneous or alternating treatment and the term “administering” is to be interpreted accordingly.

The following Examples illustrate the invention, but do not limit it.

TABLE 1
AMPA/KA receptor antagonists (parent compounds)
The invention provides prodrugs of AMPA/KA receptor
antagonists of formula (IIA) listed below in Table 1.

| | | | (IIA) |
|---|---|---|---|
| 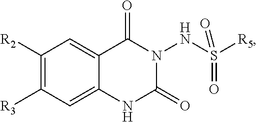 wherein R₂, R₃ and R₅ is as defined in Table 1. | | | |
| Example | | | |
| No | R₂ | R₃ | R₅ |
| 1.0 | 2-methyl-2H-pyrazol-3-yl | trifluoromethyl | methyl |
| 2.0 | 2-methyl-2H-pyrazol-3-yl | ethyl | methyl |
| 3.0 | 2-methyl-2H-pyrazol-3-yl | isopropyl | methyl |
| 4.0 | imidazol-1-yl | trifluoromethyl | methyl |
| 5.0 | 2-isopropyl-2H-pyrazol-3-yl | trifluoromethyl | methyl |
| 6.0 | 2-isopropyl-2H-pyrazol-3-yl | difluoromethyl | methyl |
| 7.0 | 2-methyl-imidazol-1-yl | trifluoromethyl | methyl |
| 8.0 | pyrrol-1-yl | trifluoromethyl | methyl |
| 9.0 | [1,2,4]triazol-4-yl | trifluoromethyl | methyl |
| 10.0 | (R)-tetrahydrofuran-2-yl | trifluoromethyl | methyl |
| 11.0 | 1-methoxy-propyl | trifluoromethyl | methyl |
| 12.0 | 1-hydroxy-propyl | trifluoromethyl | methyl |
| 13.0 | 2-ethyl-2H-pyrazol-3-yl | ethyl | methyl |
| 14.0 | 2-ethyl-2H-pyrazol-3-yl | difluoromethyl | methyl |
| 15.0 | 2-ethyl-2H-pyrazol-3-yl | trifluoromethyl | methyl |

The synthesis of Examples 1.0, 2.0, 3.0, 4.0, 5.0, 6.0, 8.0, 9.0, 10.0, 12.0 and 13.0 is described in WO2006108591 and WO2006010591, respectively.

TABLE 1A
AMPA/KA receptor antagonists (parent compounds)
The invention further provides prodrugs of AMPA/KA receptor
antagonists being 2,4-dioxo-1,4-dihydro-2H-quinazolin-
3-yl-sulfonamide derivatives as listed below in Table 1A. AMPA-
receptor binding can be demonstrated as described for Table 2.

| Ex- | | |
|---|---|---|
| ample | | IC50 |
| No | Structure | (μM) |
| 16.0 | 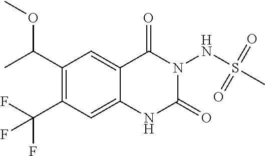 [SMILES: COC(C)c1cc2c(=O)n(NS(C)(=O)=O)c(=O)[nH]c2cc1C(F)(F)F] | 0.39 |
| 17.0 | 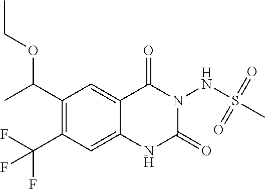 [SMILES: CCOC(C)c1cc2c(=O)n(NS(C)(=O)=O)c(=O)[nH]c2cc1C(F)(F)F] | 0.35 |
| 18.0 | 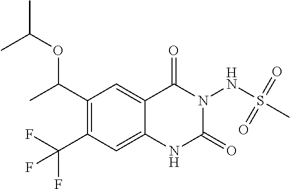 [SMILES: CC(C)OC(C)c1cc2c(=O)n(NS(C)(=O)=O)c(=O)[nH]c2cc1C(F)(F)F] | 0.32 |
| 19.0 | 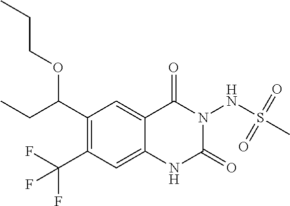 [SMILES: CCCOC(CC)c1cc2c(=O)n(NS(C)(=O)=O)c(=O)[nH]c2cc1C(F)(F)F] | 0.52 |
| 20.0 | 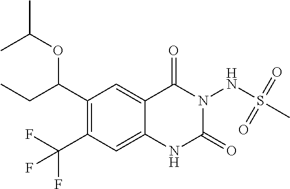 [SMILES: CCC(OC(C)C)c1cc2c(=O)n(NS(C)(=O)=O)c(=O)[nH]c2cc1C(F)(F)F] | 0.38 |
| 21.0 | 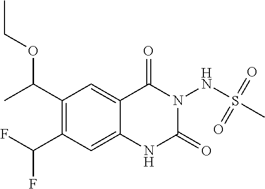 [SMILES: CCOC(C)c1cc2c(=O)n(NS(C)(=O)=O)c(=O)[nH]c2cc1C(F)F] | 0.90 |
| 22.0 | 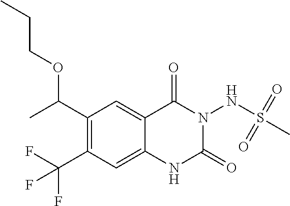 [SMILES: CCCOC(C)c1cc2c(=O)n(NS(C)(=O)=O)c(=O)[nH]c2cc1C(F)(F)F] | 0.98 |
| 23.0 | 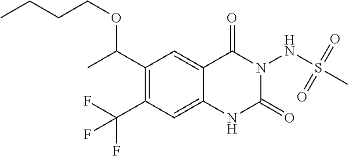 [SMILES: CCCCOC(C)c1cc2c(=O)n(NS(C)(=O)=O)c(=O)[nH]c2cc1C(F)(F)F] | 0.76 |
| 24.0 | 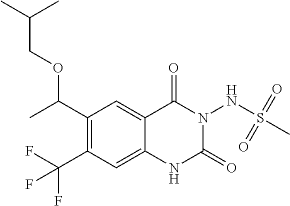 [SMILES: CC(C)COC(C)c1cc2c(=O)n(NS(C)(=O)=O)c(=O)[nH]c2cc1C(F)(F)F] | 1.81 |
| 25.0 | 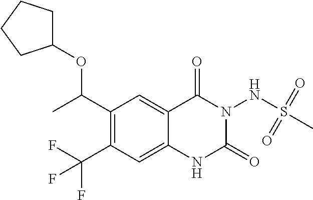 [SMILES: CC(OC1CCCC1)c1cc2c(=O)n(NS(C)(=O)=O)c(=O)[nH]c2cc1C(F)(F)F] | 0.54 |
| 26.0 | 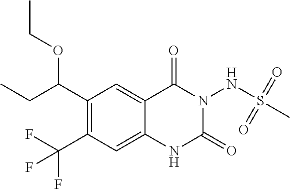 [SMILES: CCOC(CC)c1cc2c(=O)n(NS(C)(=O)=O)c(=O)[nH]c2cc1C(F)(F)F] | 0.51 |
| 27.0 | 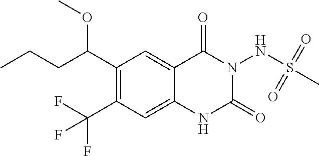 [SMILES: CCCC(OC)c1cc2c(=O)n(NS(C)(=O)=O)c(=O)[nH]c2cc1C(F)(F)F] | 0.72 |
| 28.0 | 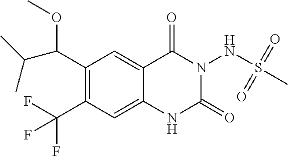 [SMILES: COC(c1cc2c(=O)n(NS(C)(=O)=O)c(=O)[nH]c2cc1C(F)(F)F)C(C)C] | 1.04 |
| 29.0 | 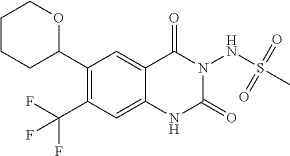 [SMILES: CS(=O)(=O)Nn1c(=O)[nH]c2cc(C(F)(F)F)c(C3CCCCO3)cc2c1=O] | 1.08 |
| 30.0 | 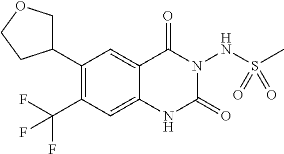 [SMILES: CS(=O)(=O)Nn1c(=O)[nH]c2cc(C(F)(F)F)c(C3CCOC3)cc2c1=O] | 0.22 |
| 31.0 | 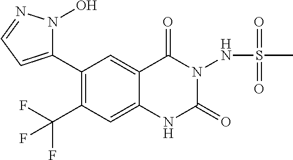 [SMILES: CS(=O)(=O)Nn1c(=O)[nH]c2cc(C(F)(F)F)c(-c3ccnn3O)cc2c1=O] | 0.14 |
| 32.0 | 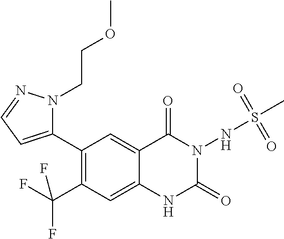 [SMILES: COCCn1nccc1-c1cc2c(=O)n(NS(C)(=O)=O)c(=O)[nH]c2cc1C(F)(F)F] | 0.31 |
| 33.0 | 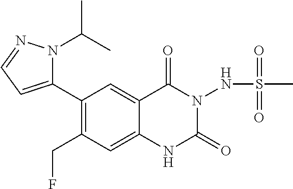 [SMILES: CC(C)n1nccc1-c1cc2c(=O)n(NS(C)(=O)=O)c(=O)[nH]c2cc1CF] | 0.44 |
| 34.0 | 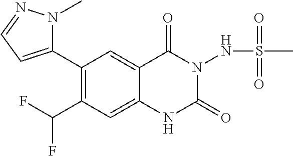 [SMILES: Cn1nccc1-c1cc2c(=O)n(NS(C)(=O)=O)c(=O)[nH]c2cc1C(F)F] | 0.26 |
| 35.0 | 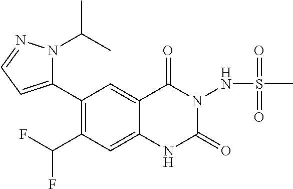 [SMILES: CC(C)n1nccc1-c1cc2c(=O)n(NS(C)(=O)=O)c(=O)[nH]c2cc1C(F)F] | 0.11 |
| 36.0 | 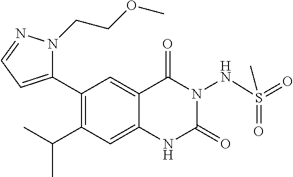 [SMILES: COCCn1nccc1-c1cc2c(=O)n(NS(C)(=O)=O)c(=O)[nH]c2cc1C(C)C] | 0.32 |
| 37.0 | 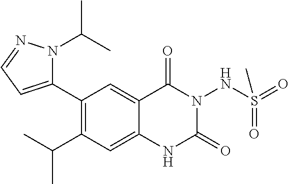 [SMILES: CC(C)c1cc2[nH]c(=O)n(NS(C)(=O)=O)c(=O)c2cc1-c1ccnn1C(C)C] | 0.43 |
| 38.0 | 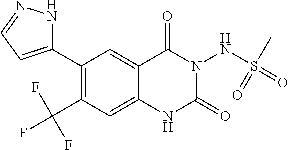 [SMILES: CS(=O)(=O)Nn1c(=O)[nH]c2cc(C(F)(F)F)c(-c3ccn[nH]3)cc2c1=O] | 0.47 |
| 39.0 | 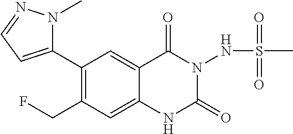 [SMILES: Cn1nccc1-c1cc2c(=O)n(NS(C)(=O)=O)c(=O)[nH]c2cc1CF] | 0.77 |
| 40.0 | 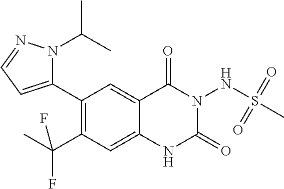 [SMILES: CC(C)n1nccc1-c1cc2c(=O)n(NS(C)(=O)=O)c(=O)[nH]c2cc1C(C)(F)F] | 0.21 |
| 41.0 | 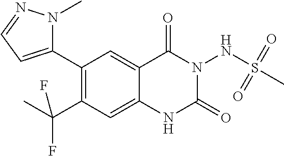 [SMILES: Cn1nccc1-c1cc2c(=O)n(NS(C)(=O)=O)c(=O)[nH]c2cc1C(C)(F)F] | 0.58 |
| 42.0 | 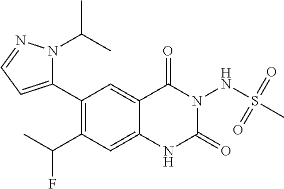 [SMILES: CC(F)c1cc2[nH]c(=O)n(NS(C)(=O)=O)c(=O)c2cc1-c1ccnn1C(C)C] | 0.22 |
| 43.0 | 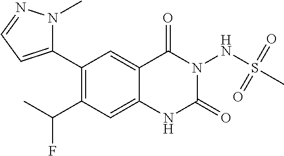 [SMILES: CC(F)c1cc2[nH]c(=O)n(NS(C)(=O)=O)c(=O)c2cc1-c1ccnn1C] | 0.70 |
| 44.0 | 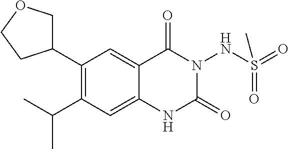 [SMILES: CC(C)c1cc2[nH]c(=O)n(NS(C)(=O)=O)c(=O)c2cc1C1CCOC1] | 2.20 |
| 45.0 | 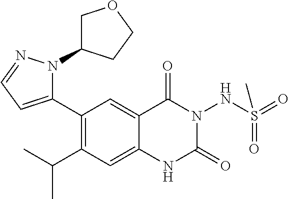 [SMILES: CC(C)c1cc2[nH]c(=O)n(NS(C)(=O)=O)c(=O)c2cc1-c1ccnn1[C@@H]1CCOC1] | 0.56 |
| 46.0 | 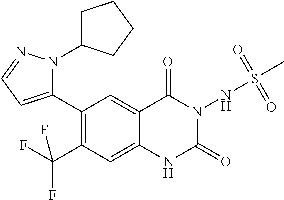 [SMILES: CS(=O)(=O)Nn1c(=O)[nH]c2cc(C(F)(F)F)c(-c3ccnn3C3CCCC3)cc2c1=O] | 1.20 |
| 47.0 | 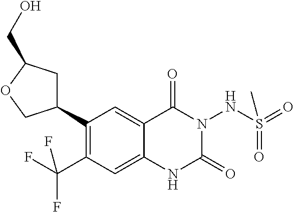 [SMILES: CS(=O)(=O)Nn1c(=O)[nH]c2cc(C(F)(F)F)c([C@H]3CO[C@@H](CO)C3)cc2c1=O] | 0.37 |
| 48.0 | 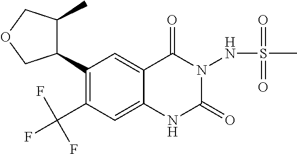 [SMILES: C[C@@H]1COC[C@@H]1c1cc2c(=O)n(NS(C)(=O)=O)c(=O)[nH]c2cc1C(F)(F)F] | 0.52 |
| 49.0 | 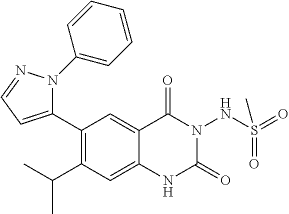 [SMILES: CC(C)c1cc2[nH]c(=O)n(NS(C)(=O)=O)c(=O)c2cc1-c1ccnn1-c1ccccc1] | 3.50 |
| 50.0 | 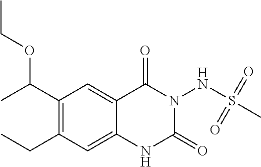 [SMILES: CCOC(C)c1cc2c(=O)n(NS(C)(=O)=O)c(=O)[nH]c2cc1CC] | 1.85 |
| 51.0 | 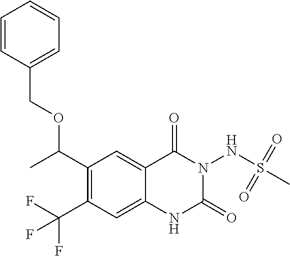 [SMILES: CC(OCc1ccccc1)c1cc2c(=O)n(NS(C)(=O)=O)c(=O)[nH]c2cc1C(F)(F)F] | 0.78 |
| 52.0 | 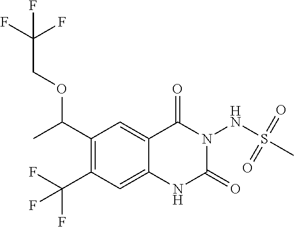 [SMILES: CC(OCC(F)(F)F)c1cc2c(=O)n(NS(C)(=O)=O)c(=O)[nH]c2cc1C(F)(F)F] | 1.12 |

### ABBREVIATIONS

AcOH acetic acid
AUC0-24 h area under the concentration curve for all measured time points (last time point: 24 h)
Boc tert-butoxycarbonyl
Cmax maximal concentration
d day(s)
DCM dichloromethane
DIC dicyclohexylcarbodiimide
DMF dimethylformamide
DMSO dimethylsulfoxide
ED50 50% effect-dose
EDC 1-(3-dimethylaminopropyl)-3-ethylcarbodiimide
ESIMS electrospray ionization mass spectrometry
EtOAc ethyl acetate
Et₂O diethyl ether
h hour(s)
Hex hexane
HOBt 1-Hydroxybenzotriazole trihydrate
HPLC high pressure liquid chromatography
i.p. intra-peritoneal (administration)
LCMS liquid chromatography mass spectroscopy
LDA lithium diisopropylamide min minute(s)
NMR nuclear magnetic resonance spectrometry
p.o. per os (oral administration)
quant. quantitative
Rt retention time
rt room temperature
s.c. sub-cutaneous (administration)
THF tetrahydrofuran
TFA trifluoroacetic acid
Tmax time point of the maximal exposure
Ts Tosyl
UPLC ultra performance liquid chromatography

### Chromatography and LC-MS Methods:

1. Flash Chromatography System:

ISCO System, CombiFlash Companion; IG Instrumenten-Gesellschaft AG. Cartusch System.

2. HPLC preparative Chromatography System:

Gilson System, Configuration: 331 Pump, 332 Pump, UV/VIS-155 and GX281 FC.

3. LC-MS System (analytical):

Agilent 1100 Series

3.1. LC-MS-Method I:

Column: PHENOMENEX Gemini C18; 100A, 3.0 um, 2.0×100 mm

Eluent: Water (+0.1% TFA): acetonitrile (+0.1% TFA) from 95:5 to 5:95 in 8.0 min, hold 95% B for 1.5 min, re-equilibrate for 0.5 min

Flow rate/Temperature: 0.6 ml/min at 50° C.

3.2. LC-MS-Method II:

Column: Agilent StableBond C-18, 1.8 um, 3.0×30 mm

Eluent: Water (+0.1% TFA): acetonitrile (+0.1% TFA) from 95:5 to 5:95 in 3.0 min, hold 95% B for 1.5 min, re-equilibrate for 0.5 min

Flow rate/Temperature: 0.6-0.8 ml/min at 37° C.

3.3. LC-MS-Method III:

Column: Agilent StableBond C-18, 1.8 um, 3.0×30 mm

Eluent: Water (+0.1% TFA): acetonitrile (+0.1% TFA) from 95:5 to 5:95 in 8.0 min, hold 95% B for 1.5 min, re-equilibrate for 0.5 min

Flow rate/Temperature: 0.5 ml/min at 37° C.

3.4. LC-MS-Method IV:

Column: VWR Chromolith SpeedRod RP-18e, 3.5 um, 4.6×50 mm

Eluent: Water (+0.1% TFA): acetonitrile (+0.1% TFA) from 95:5 to 5:95 in 8.0 min, hold 95% B for 1.5 min, re-equilibrate for 0.5 min

Flow rate/Temperature: 1.0 ml/min at 37° C.

3.5. LC-MS-Method V:

Column: VWR Chromolith Performance RP-18e, 3.5 um, 3.0×100 mm

Eluent: Water (+0.1% TFA): acetonitrile (+0.1% TFA) from 95:5 to 5:95 in 8.0 min, hold 95% B for 1.5 min, re-equilibrate for 0.5 min

Flow rate/Temperature: 1.0 ml/min at 37° C. 3.6 LC-MS-Method VI

Column: Ascentis Expresse C-18, 2.7 um, 2.1×30 mm;

Eluent: Water (+0.05% formic acid+3.75 mM ammonium acetate): acetonitrile (+0.04% formic acid) equilibrate for 0.5 min, from 98:2 to 2:98 in 1.4 min, hold 98% for 0.75 min;

Flow rate/Temperature: 1.2 ml/min at 50° C.

3.7 LC-MS-Method VII

Column: Zorbax SB-C18, 1.8 um, 3.0×30 mm;

Eluent: Water (+0.05% TFA): acetonitrile (+0.05% TFA) from 90:10 to 0:100 in 3.25 min, hold 100% B for 0.75 min, re-equilibrate for 0.25 min;

Flow rate/Temperature: 0.7 ml/min at 35° C.

3.8 LC-MS-Method VIII

Column: Zorbax SB-C18, 1.8 um, 3.0×30 mm;

Eluent: Water (+0.05% TFA): acetonitrile (+0.05% TFA) from 70:30 to 0:100 in 3.25 min, hold 100% B for 0.75 min, re-equilibrate for 0.25 min;

Flow rate/Temperature: 0.7 ml/min at 35° C.

4.0 UPLC-MS System (analytical)

Waters Acquity UPLC

4.1 UPLC-MS-Method IX

Column: Water Acquity HSS T3 1.8 um, 2.1×50 mm;

Eluent: Water (+0.05% formic acid+3.75 mM ammonium acetate): acetonitrile (+0.04% formic acid) from 98:2 to 2:98 in 1.4 min, hold for 0.75 min;

Flow rate/Temperature: 1.2 ml/min at 50° C.

### Synthesis of AMPA/KA Receptor Antagonists (Parent Compounds)

### EXAMPLE 7.0

### N-[6-(2-Methyl-imidazol-1-yl)-2,4-dioxo-7-trifluoromethyl-1,4-dihydro-2H-quinazolin-3-yl]-methanesulfonamide

### a) 5-(2-Methyl-imidazol-1-yl)-2-nitro-4-trifluoromethyl-benzoic acid methyl ester

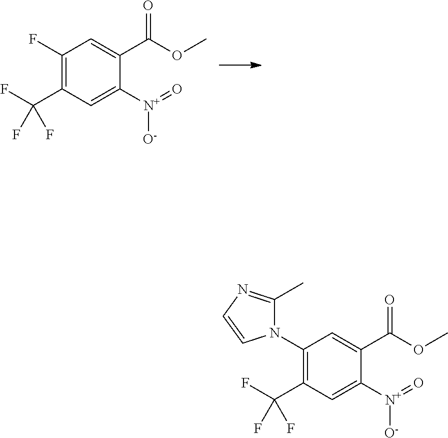 [SMILES: COC(=O)c1cc(F)c(C(F)(F)F)cc1[N+](=O)[O-] ; COC(=O)c1cc(-n2ccnc2C)c(C(F)(F)F)cc1[N+](=O)[O-]]

A suspension of 5-fluoro-2-nitro-4-trifluoromethyl-benzoic acid methyl ester (2.67 g, 10 mmol, synthesis as described in WO2006010591) and 2-methyl-1H-imidazole (1.15 g, 14 mmol) in dioxane (20 ml) was heated in the microwave oven (Biotage Initiator Microwave) at 100° C. for 30 min. Subsequently the reaction mixture was evaporated and the crude product purified by flash chromatography (ISCO Companion Flash; 40 g silica gel cartridge; dichloromethane/ethanol gradient: ethanol 0% to 10%) and the solvent evaporated to yield 5-(2-methyl-imidazol-1-yl)-2-nitro-4-trifluoromethyl-benzoic acid methyl ester (3.14 g, 9.53 mmol, 95% yield) as beige powder. LC-MS at 254 nm; M+H 330; Rt 5.611 min (method: LC-MS Method I). ¹H-NMR (360 MHz; DMSO-d⁶) δ ppm 2.11 (s, 3H), 3.90 (s, 3H), 6.96 (d, J=1.3 Hz, 1H), 7.31 (d, J=1.3 Hz, 1H), 8.19 (s, 1H), 8.67 (s, 1H).

### b) 2-Amino-5-(2-methyl-imidazol-1-yl)-4-trifluoromethyl-benzoic acid methyl ester

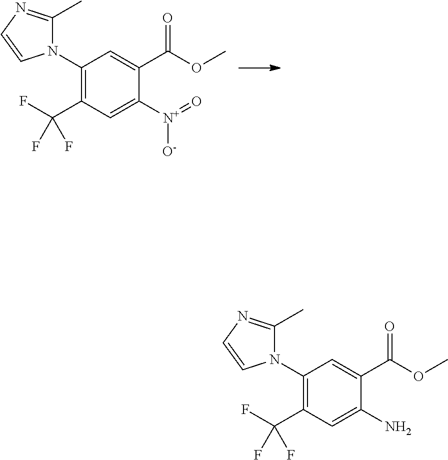 [SMILES: COC(=O)c1cc(-n2ccnc2C)c(C(F)(F)F)cc1[N+](=O)[O-] ; COC(=O)c1cc(-n2ccnc2C)c(C(F)(F)F)cc1N]

A suspension of 5-(2-methyl-imidazol-1-yl)-2-nitro-4-trifluoromethyl-benzoic acid methyl ester (3.30 g, 10 mmol) and palladium-charcoal-10% (0.5 g) in methanol (40 ml) was hydrogenated (H₂, 0.1 bar) at 23° C. for 19 hours. Subsequently the catalyst was filtered off and the filtrate evaporated. The residue was dried in vacuum at 40° C. for 60 min to yield 2-amino-5-(2-methyl-imidazol-1-yl)-4-trifluoromethyl-benzoic acid methyl ester (2.84 g, 9.5 mmol, 95% yield) as beige powder. LC-MS at 254 nm; M+H 300; Rt 2.101 min (method: LC-MS Method II). ¹H-NMR (360 MHz; DMSO-d⁶) δ ppm 2.02 (s, 3H), 3.81 (s, 3H), 6.85 (d, J=1.3 Hz, 1H), 7.08 (d, J=1.3 Hz, 1H), 7.35 (s, 1H), 7.65 (s, 1H).

### c) N-[6-(2-Methyl-imidazol-1-yl)-2,4-dioxo-7-trifluoromethyl-1,4-dihydro-2H-quinazolin-3-yl]-methanesulfonamide

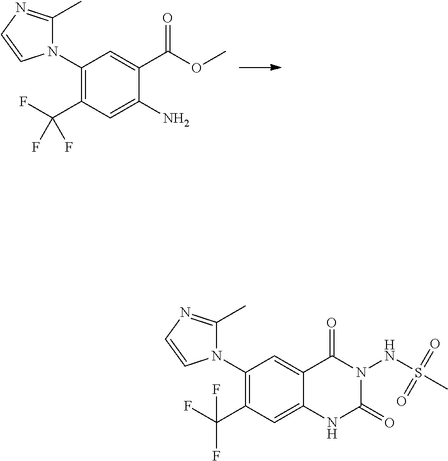 [SMILES: COC(=O)c1cc(-n2ccnc2C)c(C(F)(F)F)cc1N ; Cc1nccn1-c1cc2c(=O)n(NS(C)(=O)=O)c(=O)[nH]c2cc1C(F)(F)F]

A suspension of 2-amino-5-(2-methyl-imidazol-1-yl)-4-trifluoromethyl-benzoic acid methyl ester (2.7 g, 9.02 mmol) in tetrahydrofuran (36 ml) under nitrogen at 22° C. was treated with triphosgene (0.94 g, 3.16 mmol). The reaction mixture was stirred for 30 min at 22° C. and then cooled to 10° C. Subsequently triethylamine (1.26 ml, 9.02 mmol) was added carefully while maintaining the reaction temperature between 10° C. and 18° C. Then the yellow suspension was stirred for 180 min at 22° C. Methanesulfonylhydrazine (994 mg, 9.02 mmol) was added, and the reaction was stirred for 90 min at 22° C. Subsequently 1N aqueous sodium hydroxide (9 ml) was added slowly, and the reaction was stirred for 60 min at 22° C. Then the reaction was quenched by addition of 2N hydrochloric acid to reach pH5. The aqueous layer was extracted three times with ethyl acetate. The combined organic layers are dried over sodium sulfate, filtered, concentrated and dried under vacuum. The resulting crude product was purified by flash chromatography (ISCO Companion Flash; 80 g silica gel cartridge; dichloromethane/ethanol gradient: ethanol 0% to 10%) and the solvent evaporated to yield N-[6-(2-methyl-imidazol-1-yl)-2,4-dioxo-7-trifluoromethyl-1,4-dihydro-2H-quinazolin-3-yl]-methanesulfonamide (1.26 g, 3.13 mmol, 34.7% yield) as beige powder. LC-MS at 254 nm; M+H 404; Rt 0.491 min (method: LC-MS Method II). ¹H-NMR (600 MHz; DMSO-d⁶) δ ppm 2.05 (s, 3H), 3.20 (s, 3H), 6.92 (d, J=1 Hz, 1H), 7.19 (d, J=1 Hz, 1H), 7.69 (s, 1H), 8.01 (s, 1H).

### EXAMPLE 11.0

### N-[2,4-Dioxo-6-(1-methoxy-propyl)-7-trifluoromethyl-1,4-dihydro-2H-quinazolin-3-yl]-methanesulfonamide

### a) 2-Amino-5-(1-methoxy-propyl)-4-trifluoromethyl-benzoic acid methyl ester

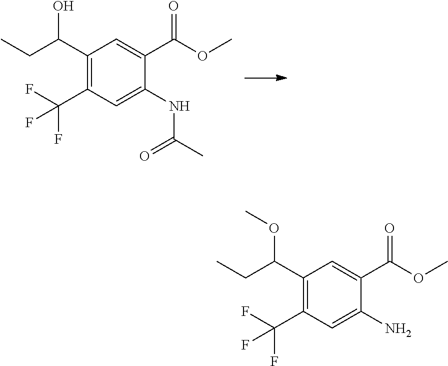 [SMILES: CCC(O)c1cc(C(=O)OC)c(NC(C)=O)cc1C(F)(F)F ; CCC(OC)c1cc(C(=O)OC)c(N)cc1C(F)(F)F]

A solution of 2-acetylamino-5-(1-hydroxy-propyl)-4-trifluoromethyl-benzoic acid methyl ester (2.1 g, 6.58 mmol) in methanol (10 ml) was treated with para-toluenesulfonic acid monohydrate (375 mg, 1.97 mmol), and the reaction was stirred for 72 hours at 22° C. The reaction mixture was then diluted with ethyl acetate and saturated aqueous NaHCO₃. The organic layer was separated, dried over MgSO4, filtered, and concentrated in vacuo. The residue was purified by flash chromatography (500 g, EtOAc/hexanes (1:99→1:4)) to give 2-amino-5-(1-methoxy-propyl)-4-trifluoromethyl-benzoic acid methyl ester (1.00 g, 51.7% yield) as white crystals. ¹H-NMR (600 MHz; DMSO-d⁶) δ ppm 0.85 (t, J=7.3 Hz, 3H), 1.57 (m, 2H), 3.05 (s, 3H), 3.83 (s, 3H), 4.18 (m, 1H), 6.94 (s, 2H), 7.16 (s, 1H), 7.90 (s, 1H).

### b) N-[2,4-Dioxo-6-(1-methoxy-propyl)-7-trifluoromethyl-1,4-dihydro-2H-quinazolin-3-yl]-methanesulfonamide

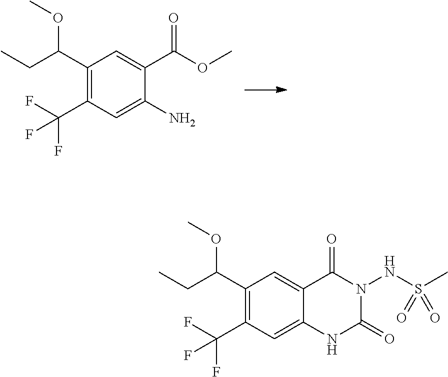 [SMILES: CCC(OC)c1cc(C(=O)OC)c(N)cc1C(F)(F)F ; CCC(OC)c1cc2c(=O)n(NS(C)(=O)=O)c(=O)[nH]c2cc1C(F)(F)F]

A solution of 2-amino-5-(1-methoxy-propyl)-4-trifluoromethyl-benzoic acid methyl ester (5.2 g, 17.85 mmol) in THF (120 ml) under argon at 22° C. was treated with triphosgene (1.85 g, 6.25 mmol). Triethylamine (2.74 ml, 19.64 mmol) was added then, and the reaction (yellow suspension) was stirred for 1 h at 22° C. Methanesulfonhydrazine (2.16 g, 19.64 mmol) was added, and the reaction was stirred for 90 min at 22° C. 2N aqueous NaOH was added slowly, and the reaction was stirred for 1 h at 22° C. The reaction was quenched by addition of brine (50 ml) and water (30 ml), and diluted with EtOAc (100 ml). The organic layer was separated and dried over Na2SO4, filtered, and concentrated in vacuo. The residue was purified by flash chromatography (125 g, CH₂Cl₂/EtOH (98:2)) to give N-[2,4-dioxo-6-(1-methoxy-propyl)-7-trifluoromethyl-1,4-dihydro-2H-quinazolin-3-yl]-methanesulfonamide (5.60 g, 79% yield) as a foam. ¹H-NMR (600 MHz; DMSO-d⁶) δ ppm 0.90 (t, J=7.5 Hz, 3H), 1.64 (m, 2H), 3.11 (s, 3H), 3.16 (s, 3H), 4.36 (m, 1H), 7.54 (s, 1H), 8.14 (s, 1H), 10.39 (s, 1H), 11.99 (s, 1H).

### EXAMPLE 14.0

### N-[7-Difluoromethyl-6-(2-ethyl-2H-pyrazol-3-yl)-2,4-dioxo-1,4-dihydro-2H-quinazolin-3-yl]-methanesulfonamide

### a) 2-Amino-4-difluoromethyl-5-(2-ethyl-2H-pyrazol-3-yl)-benzoic acid methyl ester

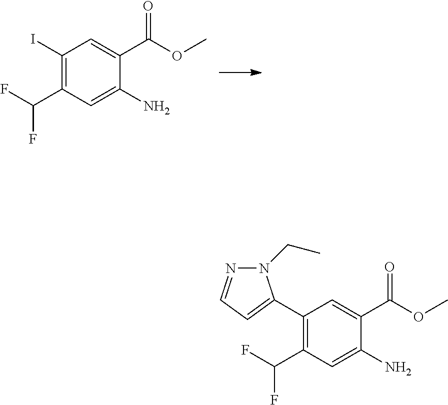 [SMILES: COC(=O)c1cc(I)c(C(F)F)cc1N ; CCn1nccc1-c1cc(C(=O)OC)c(N)cc1C(F)F]

To a solution of 2-amino-4-difluoromethyl-5-iodo-benzoic acid methyl ester (4.05 g, 12.38 mmol; synthesis as described in WO2006108591) in dioxane (50 mL), subsequently 1-ethyl-5-tributylstannanyl-1H-pyrazole (5.25 g, 13.62 mmol) and Pd(dppf)Cl2 (0.906 g, 1.238 mmol) was added. The reaction mixture was stirred at 100° C. for 24 hours. Then the solvent was evaporated and the residue suspended in ethyl acetate and filtered through hyflo. The solvent was evaporated. The crude product was washed with heptane and subsequently vacuum-dried to yield 2-amino-4-difluoromethyl-5-(2-ethyl-2H-pyrazol-3-yl)-benzoic acid methyl ester (4.28 g, 10.87 mmol; 88% yield; purity 75%); ¹H-NMR (360 MHz; DMSO-d⁶) δ ppm 1.22 (t, 3H), 3.83 (s, 3H), 3.89 (q, 2H), 6.64 (t, 1H), 7.12 (d, 1H), 7.21 (s, 1H), 7.52 (s, 1H), 7.66 (s, 1H).

### b) N-[7-Difluoromethyl-6-(2-ethyl-2H-pyrazol-3-yl)-2,4-dioxo-1,4-dihydro-2H-quinazolin-3-yl]-methanesulfonamide

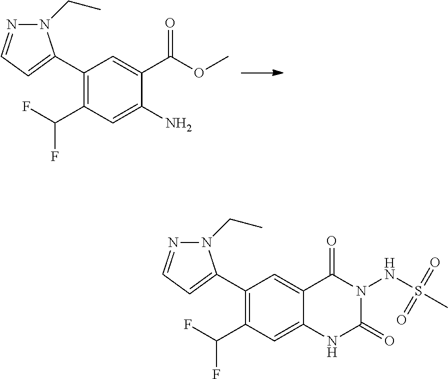 [SMILES: CCn1nccc1-c1cc(C(=O)OC)c(N)cc1C(F)F ; CCn1nccc1-c1cc2c(=O)n(NS(C)(=O)=O)c(=O)[nH]c2cc1C(F)F]

A suspension of 2-amino-4-difluoromethyl-5-(2-ethyl-2H-pyrazol-3-yl)-benzoic acid methyl ester (1.23 g, 3.12 mmol) in tetrahydrofuran (30 ml) under nitrogen at 22° C. was treated with triphosgene (0.306 g, 1.031 mmol). The reaction mixture was stirred for 30 min at 22° C. and then cooled to 10° C. Subsequently triethylamine (0.316 g, 3.12 mmol) was added carefully while maintaining the reaction temperature between 10° C. and 18° C. Then the yellow suspension was stirred for 180 min at 22° C. Methanesulfonylhydrazine (0.344 g, 3.12 mmol) was added, and the reaction was stirred for 90 min at 22° C. Subsequently 1N aqueous sodium hydroxide (5.62 ml) was added slowly, and the reaction was stirred for 30 min at 22° C. Then the reaction was quenched by addition of 2N hydrochloric acid to reach pH5. The aqueous layer was extracted three times with ethyl acetate. The combined organic layers are dried over sodium sulfate, filtered, concentrated and dried under vacuum. The resulting crude product was purified by flash chromatography (ISCO Companion Flash; 80 g silica gel cartridge; dichloromethane/methanol gradient: methanol 0% to 5%) and the solvent evaporated to yield N-[7-difluoromethyl-6-(2-ethyl-2H-pyrazol-3-yl)-2,4-dioxo-1,4-dihydro-2H-quinazolin-3-yl]-methanesulfonamide (685 mg, 1.715 mmol, 54.9% yield) as beige powder. LC-MS at 254 nm; M+H 400; Rt 3.678 min (LC-MS Method V). ¹H-NMR (600 MHz; DMSO-d⁶) δ ppm 1.18 (t, 3H), 3.19 (s, 3H), 3.91 (q, 2H), 6.36 (s, 1H), 6.84 (t, 1H), 7.58 (2s, 2H), 7.91 (s, 1H), 12.10 (s, 1H).

### EXAMPLE 15.0

### N-[6-(2-Ethyl-2H-pyrazol-3-yl)-2,4-dioxo-7-trifluoromethyl-1,4-dihydro-2H-quinazolin-3-yl]-methanesulfonamide

### a) 2-Amino-5-(2-ethyl-2H-pyrazol-3-yl)-4-trifluoromethyl-benzoic acid methyl ester

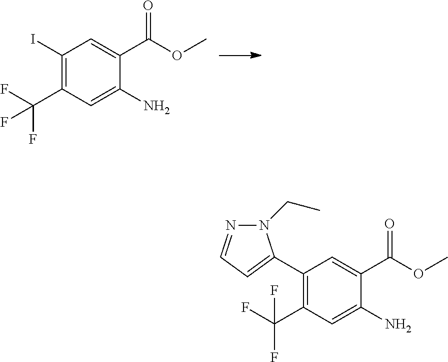 [SMILES: COC(=O)c1cc(I)c(C(F)(F)F)cc1N ; CCn1nccc1-c1cc(C(=O)OC)c(N)cc1C(F)(F)F]

To a solution of 2-amino-4-difluoromethyl-5-iodo-benzoic acid methyl ester (6.90 g, 20.0 mmol; synthesis as described in WO2006108591) in dioxane (80 mL), subsequently 1-ethyl-5-tributylstannanyl-1H-pyrazole (8.56 g, 22.0 mmol) and Pd(dppf)Cl2 (1.463 g, 2.0 mmol) was added. The reaction mixture was stirred at 100° C. for 28 hours. Then the solvent was evaporated and the residue suspended in ethyl acetate and filtered through hyflo. The solvent was evaporated. The crude product was washed with heptane and subsequently vacuum-dried to yield 2-amino-5-(2-ethyl-2H-pyrazol-3-yl)-4-trifluoromethyl-benzoic acid methyl ester (5.69 g, 15.44 mmol; 77% yield; purity 85%); LC-MS at 254 nm; M+H 314; Rt 2.909 min; LC-MS Method IV. ¹H-NMR (360 MHz; DMSO-d⁶) δ ppm 1.22 (t, 3H), 3.83 (s, 3H), 3.85 (q, 2H), 7.23 (s, 1H), 7.36 (s, 1H), 7.49 (s, 1H), 7.67 (s, 1H).

### b) N-[6-(2-Ethyl-2H-pyrazol-3-yl)-2,4-dioxo-7-trifluoromethyl-1,4-dihydro-2H-quinazolin-3-yl]-methanesulfonamide

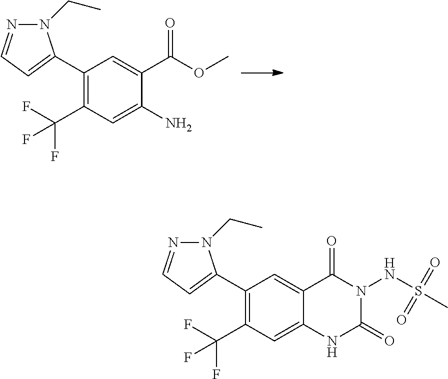 [SMILES: CCn1nccc1-c1cc(C(=O)OC)c(N)cc1C(F)(F)F ; CCn1nccc1-c1cc2c(=O)n(NS(C)(=O)=O)c(=O)[nH]c2cc1C(F)(F)F]

A suspension of 2-amino-5-(2-ethyl-2H-pyrazol-3-yl)-4-trifluoromethyl-benzoic acid methyl ester (1.80 g, 4.88 mmol) in tetrahydrofuran (40 ml) under nitrogen at 22° C. was treated with triphosgene (0.478 g, 1.612 mmol). The reaction mixture was stirred for 30 min at 22° C. and then cooled to 10° C. Subsequently triethylamine (0.494 g, 4.88 mmol) was added carefully while maintaining the reaction temperature between 10° C. and 18° C. Then the yellow suspension was stirred for 180 min at 22° C. Methanesulfonylhydrazine (0.538 g, 4.88 mmol) was added, and the reaction was stirred for 30 min at 22° C. Subsequently 1N aqueous sodium hydroxide (8.79 ml) was added slowly, and the reaction was stirred for 30 min at 22° C. Then the reaction was quenched by addition of 2N hydrochloric acid to reach pH5. The aqueous layer was extracted three times with ethyl acetate. The combined organic layers are dried over sodium sulfate, filtered, concentrated and dried under vacuum. The resulting crude product was purified by flash chromatography (ISCO Companion Flash; 80 g silica gel cartridge; dichloromethane/methanol gradient: methanol 0% to 5%) and the solvent evaporated to yield N-[6-(2-ethyl-2H-pyrazol-3-yl)-2,4-dioxo-7-trifluoromethyl-1,4-dihydro-2H-quinazolin-3-yl]-methanesulfonamide (1.47 g, 3.45 mmol, 70.7% yield) as yellow powder. LC-MS at 254 nm; M+H 418; Rt 3.989 min (LC-MS Method V). ¹H-NMR (600 MHz; DMSO-d₆) δ ppm 1.21 (t, 3H), 3.20 (s, 3H), 3.91 (q, 2H), 6.31 (s, 1H), 7.56 (d, 1H), 7.67 (s, 1H), 7.93 (s, 1H), 12.20 (broad s, 1H).

### Synthesis of Prodrugs of AM PA/KA Receptor Antagonists

### EXAMPLE 1.01

### N-[6-(2-Methyl-2H-pyrazol-3-yl)-2,4-dioxo-7-trifluoromethyl-1,4-dihydro-2H-guinazolin-3-yl]-N-pentanoyl-methanesulfonamide

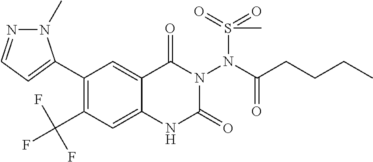 [SMILES: CCCCC(=O)N(n1c(=O)[nH]c2cc(C(F)(F)F)c(-c3ccnn3C)cc2c1=O)S(C)(=O)=O]

N-[6-(2-methyl-2H-pyrazol-3-yl)-2,4-dioxo-7-trifluoromethyl-1,4-dihydro-2H-quinazolin-3-yl]-methanesulfonamide (202 mg, 0.5 mmol) is dissolved in pyridine (2 ml) at room temperature (22° C.). Sequentially pentanoyl chloride (60.3 mg, 0.5 mmol) is added and the reaction mixture is stirred at room temperature for 1 hour. Subsequently the solvent is evaporated and the crude product is purified by silica gel flash chromatography (ISCO Companion Flash; 25 g silica gel cartridge; cyclohexane/ethyl acetate gradient: ethyl acetate 0% to 100%). Then the solvent is evaporated to yield N-[6-(2-methyl-2H-pyrazol-3-yl)-2,4-dioxo-7-trifluoromethyl-1,4-dihydro-2H-quinazolin-3-yl]-N-pentanoyl-methanesulfonamide (198.4 mg, 0.407 mmol, 81% yield) as white foam. LC-MS at 254 nm; [M+H] 488; Rt 6.682 min; LC-MS Method III. ¹H-NMR (600 MHz; DMSO-d⁶) δ ppm 0.85 (t, 3H), 1.25 (m, 2H), 1.51 (m, 2H), 2.46 (m, 2H), 3.60 (2 s, 6H), 6.36 (d, J=1 Hz, 1H), 7.54 (d, J=1 Hz, 1H), 7.28 (s, 1H), 8.06 (s, 1H), 12.60 (s, 1H).

### EXAMPLE 1.02

### N-Isobutyryl-N-[6-(2-methyl-2H-pyrazol-3-yl)-2,4-dioxo-7-trifluoromethyl-1,4-dihydro-2H-quinazolin-3-yl]-methanesulfonamide

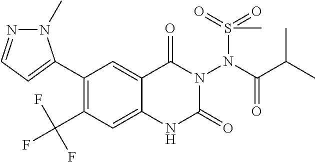 [SMILES: CC(C)C(=O)N(n1c(=O)[nH]c2cc(C(F)(F)F)c(-c3ccnn3C)cc2c1=O)S(C)(=O)=O]

Synthesis in analogy to Method C1 starting from N-[6-(2-methyl-2H-pyrazol-3-yl)-2,4-dioxo-7-trifluoromethyl-1,4-dihydro-2H-quinazolin-3-yl]-methanesulfonamide and isobutyryl chloride to yield N-isobutyryl-N-[6-(2-methyl-2H-pyrazol-3-yl)-2,4-dioxo-7-trifluoromethyl-1,4-dihydro-2H-quinazolin-3-yl]-methanesulfonamide. LC-MS at 254 nm; [M+H] 474; Rt 5.251 min; LC-MS Method III

### EXAMPLE 1.03

### N-Butyryl-N-[6-(2-methyl-2H-pyrazol-3-yl)-2,4-dioxo-7-trifluoromethyl-1,4-dihydro-2H-quinazolin-3-yl]-methanesulfonamide

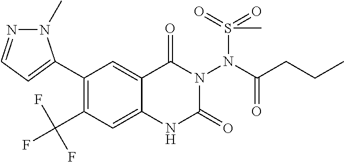 [SMILES: CCCC(=O)N(n1c(=O)[nH]c2cc(C(F)(F)F)c(-c3ccnn3C)cc2c1=O)S(C)(=O)=O]

Synthesis in analogy to Method C1 starting from N-[6-(2-methyl-2H-pyrazol-3-yl)-2,4-dioxo-7-trifluoromethyl-1,4-dihydro-2H-quinazolin-3-yl]-methanesulfonamide and butyryl chloride to yield N-butyryl-N-[6-(2-methyl-2H-pyrazol-3-yl)-2,4-dioxo-7-trifluoromethyl-1,4-dihydro-2H-quinazolin-3-yl]-methanesulfonamide. LC-MS at 254 nm; [M+H] 474; Rt 5.406 min; LC-MS Method III

### EXAMPLE 1.04

### N-Hexanoyl-N-[6-(2-methyl-2H-pyrazol-3-yl)-2,4-dioxo-7-trifluoromethyl-1,4-dihydro-2H-quinazolin-3-yl]-methanesulfonamide

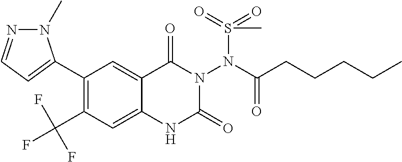 [SMILES: CCCCCC(=O)N(n1c(=O)[nH]c2cc(C(F)(F)F)c(-c3ccnn3C)cc2c1=O)S(C)(=O)=O]

Synthesis in analogy to Method C1 starting from N-[6-(2-methyl-2H-pyrazol-3-yl)-2,4-dioxo-7-trifluoromethyl-1,4-dihydro-2H-quinazolin-3-yl]-methanesulfonamide and hexanoyl chloride to yield N-hexanoyl-N-[6-(2-methyl-2H-pyrazol-3-yl)-2,4-dioxo-7-trifluoromethyl-1,4-dihydro-2H-quinazolin-3-yl]-methanesulfonamide. LC-MS at 254 nm; [M+H] 502; Rt 6.971 min; LC-MS Method III

### EXAMPLE 1.05

### Methanesulfonyl-[6-(2-methyl-2H-pyrazol-3-yl)-2,4-dioxo-7-trifluoromethyl-1,4-dihydro-2H-quinazolin-3-yl]-carbamic acid methyl ester

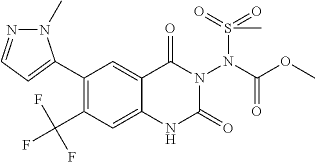 [SMILES: COC(=O)N(n1c(=O)[nH]c2cc(C(F)(F)F)c(-c3ccnn3C)cc2c1=O)S(C)(=O)=O]

Synthesis in analogy to Method C1 starting from N-[6-(2-methyl-2H-pyrazol-3-yl)-2,4-dioxo-7-trifluoromethyl-1,4-dihydro-2H-quinazolin-3-yl]-methanesulfonamide and methyl chloroformate to yield methanesulfonyl-[6-(2-methyl-2H-pyrazol-3-yl)-2,4-dioxo-7-trifluoromethyl-1,4-dihydro-2H-quinazolin-3-yl]-carbamic acid methyl ester. LC-MS at 254 nm; [M+H] 462; Rt 4.890 min; LC-MS Method III

### EXAMPLE 1.06

### Methanesulfonyl-[6-(2-methyl-2H-pyrazol-3-yl)-2,4-dioxo-7-trifluoromethyl-1,4-dihydro-2H-quinazolin-3-yl]-carbamic acid isobutyl ester

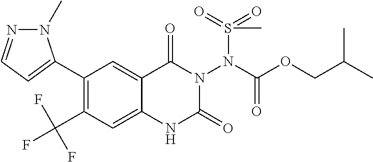 [SMILES: CC(C)COC(=O)N(n1c(=O)[nH]c2cc(C(F)(F)F)c(-c3ccnn3C)cc2c1=O)S(C)(=O)=O]

Synthesis in analogy to Method C1 starting from N-[6-(2-methyl-2H-pyrazol-3-yl)-2,4-dioxo-7-trifluoromethyl-1,4-dihydro-2H-quinazolin-3-yl]-methanesulfonamide and isobutyl chloroformate to yield methanesulfonyl-[6-(2-methyl-2H-pyrazol-3-yl)-2,4-dioxo-7-trifluoromethyl-1,4-dihydro-2H-quinazolin-3-yl]-carbamic acid isobutyl ester. LC-MS at 254 nm; [M+H] 504; Rt 5.804 min; LC-MS Method III

### EXAMPLE 1.07

### Methanesulfonyl-[6-(2-methyl-2H-pyrazol-3-yl)-2,4-dioxo-7-trifluoromethyl-1,4-dihydro-2H-quinazolin-3-yl]-carbamic acid 2-methoxy-ethyl ester

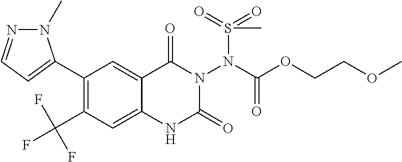 [SMILES: COCCOC(=O)N(n1c(=O)[nH]c2cc(C(F)(F)F)c(-c3ccnn3C)cc2c1=O)S(C)(=O)=O]

Synthesis in analogy to Method C1 starting from N-[6-(2-methyl-2H-pyrazol-3-yl)-2,4-dioxo-7-trifluoromethyl-1,4-dihydro-2H-quinazolin-3-yl]-methanesulfonamide and 2-methoxyethyl chloroformate to yield methanesulfonyl-[6-(2-methyl-2H-pyrazol-3-yl)-2,4-dioxo-7-trifluoromethyl-1,4-dihydro-2H-quinazolin-3-yl]-carbamic acid 2-methoxy-ethyl ester. LC-MS at 254 nm; [M+H] 506; Rt 6.990 min; LC-MS Method III

### EXAMPLE 2.01

### N-Acetyl-N-[7-ethyl-6-(2-methyl-2H-pyrazol-3-yl)-2,4-dioxo-1,4-dihydro-2H-quinazolin-3-yl]-methanesulfonamide

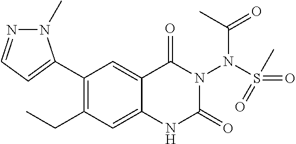 [SMILES: CCc1cc2[nH]c(=O)n(N(C(C)=O)S(C)(=O)=O)c(=O)c2cc1-c1ccnn1C]

N-[7-ethyl-6-(2-methyl-2H-pyrazol-3-yl)-2,4-dioxo-1,4-dihydro-2H-quinazolin-3-yl]-methanesulfonamide (150 mg, 0.41 mmol) was dissolved in DMF (5 ml) at room temperature (22° C.). NaH (17 mg of a 60% dispersion, 0.41 mmol) was added, and the reaction mixture was stirred for 1 h. Acetyl chloride (32 mg, 0.41 mmol) was added, and the reaction was stirred for 1 h. The reaction was then quenched by addition of brine and EtOAc. The organic solvents were separated, washed with brine, dried over Na2SO4, filtered, and concentrated in vacuo. The residue was purified by silica gel flash chromatography (20 g silica gel; toluene/CH₂Cl₂/EtOH in a ratio of 60/35/5) to yield N-acetyl-N-[7-ethyl-6-(2-methyl-2H-pyrazol-3-yl)-2,4-dioxo-1,4-dihydro-2H-quinazolin-3-yl]-methanesulfonamide (107 mg, 0.26 mmol, 64% yield) as a white solid. LC-MS at 254 nm; [M+H] 406; Rt 2.896 min; (LC-MS Method VII). ¹H-NMR (600 MHz; DMSO-d⁶) δ ppm 1.04 (t, J=7.6 Hz, 3H), 2.12 (s, 3H), 2.50 (m, 2H), 3.58 (s, 3H), 3.59 (s, 3H), 6.32 (s, 1H), 7.26 (s, 1H), 7.53 (s, 1H), 7.79 (s, 1H), 12.24 (s, 1H).

### EXAMPLE 2.02

### N-Butyryl-N-[7-ethyl-6-(2-methyl-2H-pyrazol-3-yl)-2,4-dioxo-1,4-dihydro-2H-quinazolin-3-yl]-methanesulfonamide

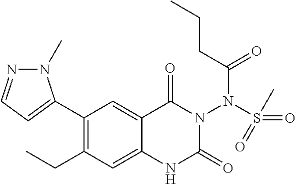 [SMILES: CCCC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and butyryl chloride to yield N-butyryl-N-[7-ethyl-6-(2-methyl-2H-pyrazol-3-yl)-2,4-dioxo-1,4-dihydro-2H-quinazolin-3-yl]-methanesulfonamide. LC-MS at 254 nm; [M+H] 434; Rt 3.198 min; (LC-MS Method VII)

### EXAMPLE 2.03

### N-[7-Ethyl-6-(2-methyl-2H-pyrazol-3-yl)-2,4-dioxo-1,4-dihydro-2H-quinazolin-3-yl]-N-hexanoyl-methanesulfonamide

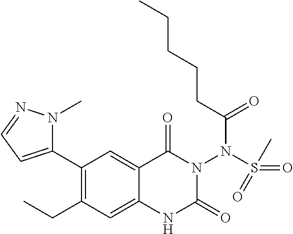 [SMILES: CCCCCC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and hexanoyl chloride to yield N-[7-ethyl-6-(2-methyl-2H-pyrazol-3-yl)-2,4-dioxo-1,4-dihydro-2H-quinazolin-3-yl]-N-hexanoyl-methanesulfonamide. LC-MS at 254 nm; [M+H] 462; Rt 3.515 min; (LC-MS Method VII)

### EXAMPLE 2.04

### Methanesulfonyl-[7-ethyl-6-(2-methyl-2H-pyrazol-3-yl)-2,4-dioxo-1,4-dihydro-2H-quinazolin-3-yl]-carbamic acid isobutyl ester

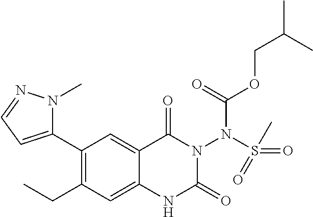 [SMILES: CCc1cc2[nH]c(=O)n(N(C(=O)OCC(C)C)S(C)(=O)=O)c(=O)c2cc1-c1ccnn1C]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and isobutyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid isobutyl ester. LC-MS at 254 nm; [M+H] 464; Rt 3.365 min; (LC-MS Method VII)

### EXAMPLE 2.05

### Methanesulfonyl-[7-ethyl-6-(2-methyl-2H-pyrazol-3-yl)-2,4-dioxo-1,4-dihydro-2H-quinazolin-3-yl]-carbamic acid 2-methoxy-ethyl ester

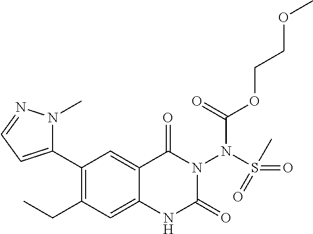 [SMILES: CCc1cc2[nH]c(=O)n(N(C(=O)OCCOC)S(C)(=O)=O)c(=O)c2cc1-c1ccnn1C]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and 2-methoxy-ethyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid 2-methoxy-ethyl ester. LC-MS at 254 nm; [M+H] 466; Rt 3.009 min; (LC-MS Method VII)

### EXAMPLE 2.06

### Methanesulfonyl-[7-ethyl-6-(2-methyl-2H-pyrazol-3-yl)-2,4-dioxo-1,4-dihydro-2H-quinazolin-3-yl]-carbamic acid propyl ester

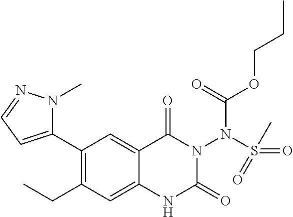 [SMILES: CCCOC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and propyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid propyl ester. LC-MS at 254 nm; [M+H] 450; Rt 3.221 min; (LC-MS Method VII)

### EXAMPLE 2.07

### Methanesulfonyl-[7-ethyl-6-(2-methyl-2H-pyrazol-3-yl)-2,4-dioxo-1,4-dihydro-2H-quinazolin-3-yl]-carbamic acid methyl ester

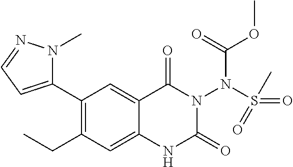 [SMILES: CCc1cc2[nH]c(=O)n(N(C(=O)OC)S(C)(=O)=O)c(=O)c2cc1-c1ccnn1C]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and methyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid methyl ester. LC-MS at 254 nm; [M+H] 422; Rt 2.951 min; (LC-MS Method VII)

### EXAMPLE 2.08

### Methanesulfonyl-[7-ethyl-6-(2-methyl-2H-pyrazol-3-yl)-2,4-dioxo-1,4-dihydro-2H-quinazolin-3-yl]-carbamic acid ethyl ester

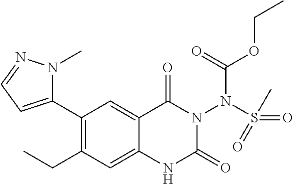 [SMILES: CCOC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and ethyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid ethyl ester. LC-MS at 254 nm; [M+H] 436; Rt 3.079 min; (LC-MS Method VII)

### EXAMPLE 2.09

### Methanesulfonyl-[7-ethyl-6-(2-methyl-2H-pyrazol-3-yl)-2,4-dioxo-1,4-dihydro-2H-quinazolin-3-yl]-carbamic acid butyl ester

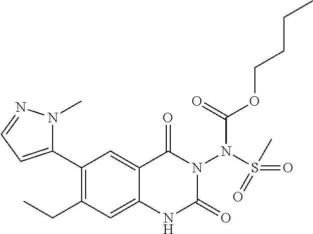 [SMILES: CCCCOC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and butyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid butyl ester. LC-MS at 254 nm; [M+H] 464; Rt 3.371 min; (LC-MS Method VII)

### EXAMPLE 2.10

### N-[7-Ethyl-6-(2-methyl-2H-pyrazol-3-yl)-2,4-dioxo-1,4-dihydro-2H-quinazolin-3-yl]-N-propionyl-methanesulfonamide

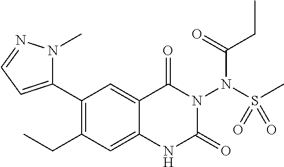 [SMILES: CCC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and propionyl chloride to yield N-[7-ethyl-6-(2-methyl-2H-pyrazol-3-yl)-2,4-dioxo-1,4-dihydro-2H-quinazolin-3-yl]-N-propionyl-methanesulfonamide. LC-MS at 254 nm; [M+H] 420; Rt 0.99 min; (LC-MS Method VI)

### EXAMPLE 2.11

### N-[7-Ethyl-6-(2-methyl-2H-pyrazol-3-yl)-2,4-dioxo-1,4-dihydro-2H-quinazolin-3-yl]-N-pentanoyl-methanesulfonamide

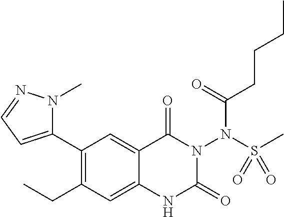 [SMILES: CCCCC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and pentanoyl chloride to yield N-[7-ethyl-6-(2-methyl-2H-pyrazol-3-yl)-2,4-dioxo-1,4-dihydro-2H-quinazolin-3-yl]-N-pentanoyl-methanesulfonamide. LC-MS at 254 nm; [M+H] 448; Rt 1.08 min; (LC-MS Method VI)

### EXAMPLE 2.12

### Methanesulfonyl-[7-ethyl-6-(2-methyl-2H-pyrazol-3-yl)-2,4-dioxo-1,4-dihydro-2H-quinazolin-3-yl]-carbamic acid isopropyl ester

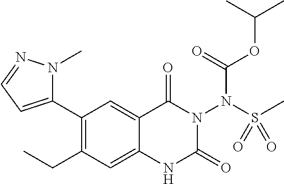 [SMILES: CCc1cc2[nH]c(=O)n(N(C(=O)OC(C)C)S(C)(=O)=O)c(=O)c2cc1-c1ccnn1C]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and isopropyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid isopropyl ester. LC-MS at 254 nm; [M+H] 450; Rt 1.04 min; (LC-MS Method VI)

### EXAMPLE 2.13

### Methanesulfonyl-[7-ethyl-6-(2-methyl-2H-pyrazol-3-yl)-2,4-dioxo-1,4-dihydro-2H-quinazolin-3-yl]-carbamic acid pentyl ester

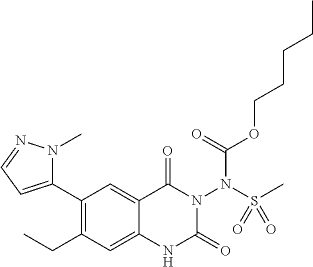 [SMILES: CCCCCOC(=O)N(n1c(=O)[nH]c2cc(CC)c(-c3ccnn3C)cc2c1=O)S(C)(=O)=O]

Synthesis in analogy to Method B1 starting from N-[7-ethyl-6-(2-methyl-2H-pyrazol-3-yl)-2,4-dioxo-1,4-dihydro-2H-quinazolin-3-yl]-methanesulfonamide and pentyl chloroformate to yield methanesulfonyl-[7-ethyl-6-(2-methyl-2H-pyrazol-3-yl)-2,4-dioxo-1,4-dihydro-2H-quinazolin-3-yl]-carbamic acid pentyl ester. LC-MS at 254 nm; [M+NH₄] 495; Rt 1.09 min; (LC-MS Method VI)

### EXAMPLE 2.14

### N-Acetyl-N-[1-acetyl-7-ethyl-6-(2-methyl-2H-pyrazol-3-yl)-2,4-dioxo-1,4-dihydro-2H-quinazolin-3-yl]-methanesulfonamide

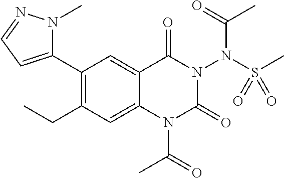 [SMILES: CCc1cc2c(cc1-c1ccnn1C)c(=O)n(N(C(C)=O)S(C)(=O)=O)c(=O)n2C(C)=O]

Synthesis in analogy to Method A1 starting from N-[7-ethyl-6-(2-methyl-2H-pyrazol-3-yl)-2,4-dioxo-1,4-dihydro-2H-quinazolin-3-yl]-methanesulfonamide and acetyl chloride to yield N-acetyl-N-[1-acetyl-7-ethyl-6-(2-methyl-2H-pyrazol-3-yl)-2,4-dioxo-1,4-dihydro-2H-quinazolin-3-yl]-methanesulfonamide. LC-MS at 254 nm; [M+H] 448; Rt 1.02 min; (LC-MS Method VI)

### EXAMPLE 3.01

### N-Acetyl-N-[7-isopropyl-6-(2-methyl-2H-pyrazol-3-yl)-2,4-dioxo-1,4-dihydro-2H-quinazolin-3-yl]-methanesulfonamide

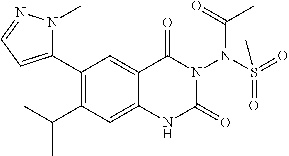 [SMILES: CC(=O)N(n1c(=O)[nH]c2cc(C(C)C)c(-c3ccnn3C)cc2c1=O)S(C)(=O)=O]

N-[7-isopropyl-6-(2-methyl-2H-pyrazol-3-yl)-2,4-dioxo-1,4-dihydro-2H-quinazolin-3-yl]-methanesulfonamide (755.0 mg, 2.0 mmol) is suspended in triethylamine (0.837 mL) and dry dichloromethane (2 mL) at room temperature (22° C.). Sequentially acetyl chloride (0.178 mL, 6 mmol) is added while keeping the reaction temperature between 3-8° C. Then the reaction mixture is stirred at room temperature for 1 hours. Subsequently the crude reaction mixture is poured into a flask containing saturated sodium hydrogen carbonate solution and extracted the crude product with methylene chloride. The combined organic layers were dried over Na2SO4, filtered, concentrated and subjected to silica gel flash chromatography (ISCO CombiFlash); 12 g silica gel cartridge; cyclohexane/ethyl acetate gradient: ethyl acetate 0% to 100%. Then the solvents are evaporated to yield N-acetyl-N-[7-isopropyl-6-(2-methyl-2H-pyrazol-3-yl)-2,4-dioxo-1,4-dihydro-2H-quinazolin-3-yl]-methanesulfonamide (805.0 mg, 1.92 mmol, 96%) as beige foam. LC-MS at 254 nm; [M+H] 420; Rt 6.388 min; LC-MS Method I. ¹H-NMR (600 MHz; DMSO-d⁶) δ ppm 1.12 (2 d, 6H), 2.15 (broad s, 3H), 2.74 (m, 1H), 3.57 (2 s, 6H), 6.30 (d, J=1 Hz, 1H), 7.33 (s, 1H), 7.50 (d, J=1 Hz, 1H), 7.80 (s, 1H), 12.20 (s, 1H).

### EXAMPLE 3.02

### [7-Isopropyl-6-(2-methyl-2H-pyrazol-3-yl)-2,4-dioxo-1,4-dihydro-2H-quinazolin-3-yl]-methanesulfonyl-carbamic acid 2-methoxy-ethyl ester

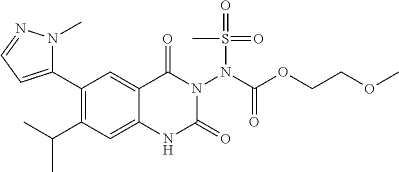 [SMILES: COCCOC(=O)N(n1c(=O)[nH]c2cc(C(C)C)c(-c3ccnn3C)cc2c1=O)S(C)(=O)=O]

Synthesis in analogy to Method B1 starting from N-[7-isopropyl-6-(2-methyl-2H-pyrazol-3-yl)-2,4-dioxo-1,4-dihydro-2H-quinazolin-3-yl]-methanesulfonamide and 2-methoxyethyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid 2-methoxy-ethyl ester. LC-MS at 254 nm; [M+H] 480; Rt 5.852 min; LC-MS Method I

### EXAMPLE 3.03

### [7-Isopropyl-6-(2-methyl-2H-pyrazol-3-yl)-2,4-dioxo-1,4-dihydro-2H-quinazolin-3-yl]-methanesulfonyl-carbamic acid isobutyl ester

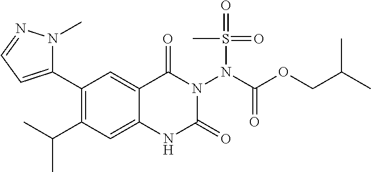 [SMILES: CC(C)COC(=O)N(n1c(=O)[nH]c2cc(C(C)C)c(-c3ccnn3C)cc2c1=O)S(C)(=O)=O]

Synthesis in analogy to Method B1 starting from N-[7-isopropyl-6-(2-methyl-2H-pyrazol-3-yl)-2,4-dioxo-1,4-dihydro-2H-quinazolin-3-yl]-methanesulfonamide and isobutyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid isobutyl ester. LC-MS at 254 nm; [M+H] 478; Rt 7.246 min; LC-MS Method I

### EXAMPLE 3.04

### 3-(Isobutoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid isobutyl ester

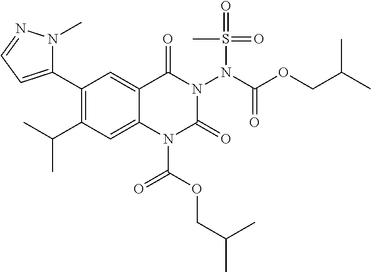 [SMILES: CC(C)COC(=O)N(n1c(=O)c2cc(-c3ccnn3C)c(C(C)C)cc2n(C(=O)OCC(C)C)c1=O)S(C)(=O)=O]

Synthesis in analogy to Method B1 starting from N-[7-isopropyl-6-(2-methyl-2H-pyrazol-3-yl)-2,4-dioxo-1,4-dihydro-2H-quinazolin-3-yl]-methanesulfonamide and isobutyl chloroformate to yield 3-(isobutoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid isobutyl ester. LC-MS at 254 nm; [M+H] 578; Rt 8.150 min; LC-MS Method I

### EXAMPLE 3.05

### [7-Isopropyl-6-(2-methyl-2H-pyrazol-3-yl)-2,4-dioxo-1,4-dihydro-2H-quinazolin-3-yl]-methanesulfonyl-carbamic acid methyl ester

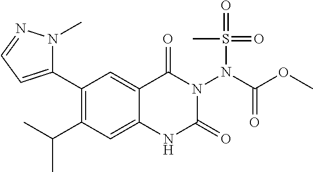 [SMILES: COC(=O)N(n1c(=O)[nH]c2cc(C(C)C)c(-c3ccnn3C)cc2c1=O)S(C)(=O)=O]

Synthesis in analogy to Method B1 starting from N-[7-isopropyl-6-(2-methyl-2H-pyrazol-3-yl)-2,4-dioxo-1,4-dihydro-2H-quinazolin-3-yl]-methanesulfonamide and methyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid methyl ester. LC-MS at 254 nm; [M+H] 436; Rt 5.767 min; LC-MS Method I

### EXAMPLE 3.06

### 4-{[7-Isopropyl-6-(2-methyl-2H-pyrazol-3-yl)-2,4-dioxo-1,4-dihydro-2H-quinazolin-3-yl]-methanesulfonyl-amino}-4-oxo-butyric acid ethyl ester

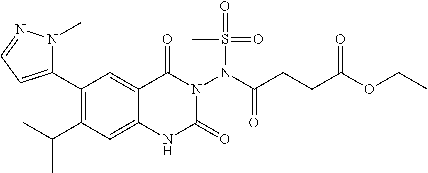 [SMILES: CCOC(=O)CCC(=O)N(n1c(=O)[nH]c2cc(C(C)C)c(-c3ccnn3C)cc2c1=O)S(C)(=O)=O]

Synthesis in analogy to Method E1 starting from N-[7-isopropyl-6-(2-methyl-2H-pyrazol-3-yl)-2,4-dioxo-1,4-dihydro-2H-quinazolin-3-yl]-methanesulfonamide and 3-chlorocarbonyl-propionic acid ethyl ester to yield 4-{[7-isopropyl-6-(2-methyl-2H-pyrazol-3-yl)-2,4-dioxo-1,4-dihydro-2H-quinazolin-3-yl]-methanesulfonyl-amino}-4-oxo-butyric acid ethyl ester. LC-MS at 254 nm; [M+H] 506; Rt 7.862 min; LC-MS Method I

### EXAMPLE 3.07

### 3-{[7-Isopropyl-6-(2-methyl-2H-pyrazol-3-yl)-2,4-dioxo-1,4-dihydro-2H-quinazolin-3-yl]-methanesulfonyl-amino}-3-oxo-propionic acid ethyl ester

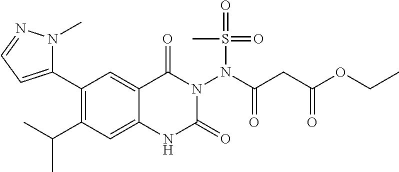 [SMILES: CCOC(=O)CC(=O)N(n1c(=O)[nH]c2cc(C(C)C)c(-c3ccnn3C)cc2c1=O)S(C)(=O)=O]

Synthesis in analogy to Method E1 starting from N-[7-isopropyl-6-(2-methyl-2H-pyrazol-3-yl)-2,4-dioxo-1,4-dihydro-2H-quinazolin-3-yl]-methanesulfonamide and ethyl malonyl chloride to yield 3-{[7-isopropyl-6-(2-methyl-2H-pyrazol-3-yl)-2,4-dioxo-1,4-dihydro-2H-quinazolin-3-yl]-methanesulfonyl-amino}-3-oxo-propionic acid ethyl ester. LC-MS at 254 nm; [M+H] 492; Rt 2.881 min; LC-MS Method II

### EXAMPLE 3.08

### 5-{[7-Isopropyl-6-(2-methyl-2H-pyrazol-3-yl)-2,4-dioxo-1,4-dihydro-2H-quinazolin-3-yl]-methanesulfonyl-amino}-5-oxo-pentanoic acid ethyl ester

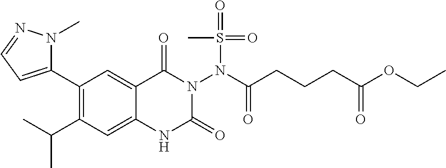 [SMILES: CCOC(=O)CCCC(=O)N(n1c(=O)[nH]c2cc(C(C)C)c(-c3ccnn3C)cc2c1=O)S(C)(=O)=O]

Synthesis in analogy to Method E1 starting from N-[7-isopropyl-6-(2-methyl-2H-pyrazol-3-yl)-2,4-dioxo-1,4-dihydro-2H-quinazolin-3-yl]-methanesulfonamide and ethyl glutaryl chloride to yield 5-{[7-isopropyl-6-(2-methyl-2H-pyrazol-3-yl)-2,4-dioxo-1,4-dihydro-2H-quinazolin-3-yl]-methanesulfonyl-amino}-5-oxo-pentanoic acid ethyl ester. LC-MS at 254 nm; [M+H] 520; Rt 2.968 min; LC-MS Method II

### EXAMPLE 3.09

### N-[7-Isopropyl-6-(2-methyl-2H-pyrazol-3-yl)-2,4-dioxo-1,4-dihydro-2H-quinazolin-3-yl]-N-pentanoyl-methanesulfonamide

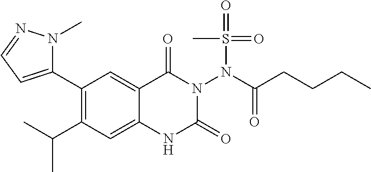 [SMILES: CCCCC(=O)N(n1c(=O)[nH]c2cc(C(C)C)c(-c3ccnn3C)cc2c1=O)S(C)(=O)=O]

Synthesis in analogy to Method C1 starting from N-[7-isopropyl-6-(2-methyl-2H-pyrazol-3-yl)-2,4-dioxo-1,4-dihydro-2H-quinazolin-3-yl]-methanesulfonamide and pentanoyl chloride to yield N-[7-isopropyl-6-(2-methyl-2H-pyrazol-3-yl)-2,4-dioxo-1,4-dihydro-2H-quinazolin-3-yl]-N-pentanoyl-methanesulfonamide. LC-MS at 254 nm; [M+H] 462; Rt 6.894 min; LC-MS Method III

### EXAMPLE 3.10

### Acetic acid 2-{[7-isopropyl-6-(2-methyl-2H-pyrazol-3-yl)-2,4-dioxo-1,4-dihydro-2H-quinazolin-3-yl]-methanesulfonyl-amino}-1,1-dimethyl-2-oxo-ethyl ester

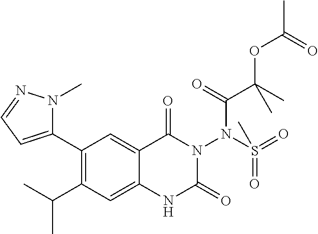 [SMILES: CC(=O)OC(C)(C)C(=O)N(n1c(=O)[nH]c2cc(C(C)C)c(-c3ccnn3C)cc2c1=O)S(C)(=O)=O]

Synthesis in analogy to Method E1 starting from N-[7-isopropyl-6-(2-methyl-2H-pyrazol-3-yl)-2,4-dioxo-1,4-dihydro-2H-quinazolin-3-yl]-methanesulfonamide and acetic acid 1-chlorocarbonyl-1-methyl-ethyl ester to yield acetic acid 2-{[7-isopropyl-6-(2-methyl-2H-pyrazol-3-yl)-2,4-dioxo-1,4-dihydro-2H-quinazolin-3-yl]-methanesulfonyl-amino}-1,1-dimethyl-2-oxo-ethyl ester. LC-MS at 254 nm; [M+H] 506; double peak Rt 7.228 and 7.365 min; LC-MS Method I

### EXAMPLE 3.11

### Acetic acid 2-[3-[(2-acetoxy-2-methyl-propionyl)-methanesulfonyl-amino]-7-isopropyl-6-(2-methyl-2H-pyrazol-3-yl)-2,4-dioxo-3,4-dihydro-2H-quinazolin-1-yl]-1,1-dimethyl-2-oxo-ethyl ester

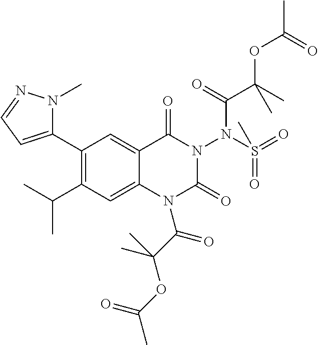 [SMILES: CC(=O)OC(C)(C)C(=O)N(n1c(=O)c2cc(-c3ccnn3C)c(C(C)C)cc2n(C(=O)C(C)(C)OC(C)=O)c1=O)S(C)(=O)=O]

Synthesis in analogy to Method E1 starting from N-[7-isopropyl-6-(2-methyl-2H-pyrazol-3-yl)-2,4-dioxo-1,4-dihydro-2H-quinazolin-3-yl]-methanesulfonamide and acetic acid 1-chlorocarbonyl-1-methyl-ethyl ester to yield acetic acid 2-[3-[(2-acetoxy-2-methyl-propionyl)-methanesulfonyl-amino]-7-isopropyl-6-(2-methyl-2H-pyrazol-3-yl)-2,4-dioxo-3,4-dihydro-2H-quinazolin-1-yl]-1,1-dimethyl-2-oxo-ethyl ester. LC-MS at 254 nm; [M+H] 634; Rt 3.872 min; LC-MS Method II

### EXAMPLE 3.12

### N-[7-Isopropyl-6-(2-methyl-2H-pyrazol-3-yl)-2,4-dioxo-1,4-dihydro-2H-quinazolin-3-yl]-N-propionyl-methanesulfonamide

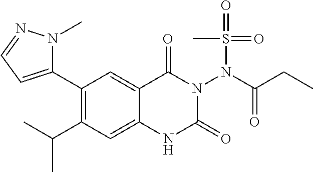 [SMILES: CCC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and propionyl chloride to yield N-[7-isopropyl-6-(2-methyl-2H-pyrazol-3-yl)-2,4-dioxo-1,4-dihydro-2H-quinazolin-3-yl]-N-propionyl-methanesulfonamide. LC-MS at 254 nm; [M+H] 434; Rt 4.465 min; LC-MS Method V

### EXAMPLE 3.13

### N-Butyryl-N-[7-isopropyl-6-(2-methyl-2H-pyrazol-3-yl)-2,4-dioxo-1,4-dihydro-2H-quinazolin-3-yl]-methanesulfonamide

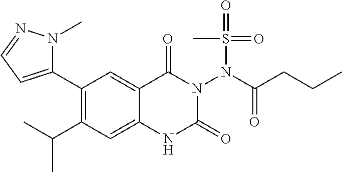 [SMILES: CCCC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and butyryl chloride to yield N-butyryl-N-[7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 448; Rt 4.791 min; LC-MS Method V

### EXAMPLE 3.14

### N-Hexanoyl-N-[7-isopropyl-6-(2-methyl-2H-pyrazol-3-yl)-2,4-dioxo-1,4-dihydro-2H-quinazolin-3-yl]-methanesulfonamide

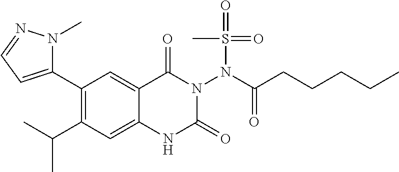 [SMILES: CCCCCC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and hexanoyl chloride to yield N-hexanoyl-N-[7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 476; Rt 5.460 min; LC-MS Method V

### EXAMPLE 3.15

### N-Decanoyl-N-[7-isopropyl-6-(2-methyl-2H-pyrazol-3-yl)-2,4-dioxo-1,4-dihydro-2H-quinazolin-3-yl]-methanesulfonamide

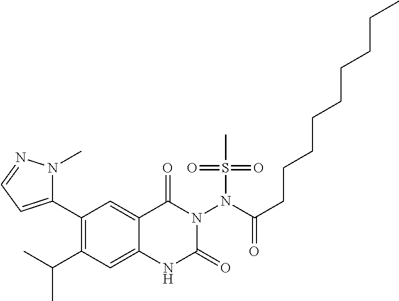 [SMILES: CCCCCCCCCC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and decanoyl chloride to yield N-decanoyl-N-[7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 532; Rt 7.063 min; LC-MS Method V

### EXAMPLE 3.16

### N-Isobutyryl-N-[7-isopropyl-6-(2-methyl-2H-pyrazol-3-yl)-2,4-dioxo-1,4-dihydro-2H-quinazolin-3-yl]-methanesulfonamide

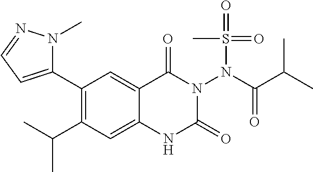 [SMILES: CC(C)C(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and isobutyryl chloride to yield N-isobutyryl-N-[7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 448; Rt 4.739 min; LC-MS Method V

### EXAMPLE 3.17

### Acetic acid 2-{[7-isopropyl-6-(2-methyl-2H-pyrazol-3-yl)-2,4-dioxo-1,4-dihydro-2H-quinazolin-3-yl]-methanesulfonyl-amino}-2-oxo-ethyl ester

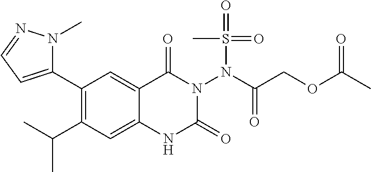 [SMILES: CC(=O)OCC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and acetic acid chlorocarbonylmethyl ester to yield acetic acid 2-{[7-isopropyl-6-(2-methyl-2H-pyrazol-3-yl)-2,4-dioxo-1,4-dihydro-2H-quinazolin-3-yl]-methanesulfonyl-amino}-2-oxo-ethyl ester. LC-MS at 254 nm; [M+H] 478; Rt 4.372 min; LC-MS Method V

### EXAMPLE 3.18

### N-[7-Isopropyl-6-(2-methyl-2H-pyrazol-3-yl)-2,4-dioxo-1,4-dihydro-2H-quinazolin-3-yl]-N-(3-methylsulfanyl-propionyl)-methanesulfonamide

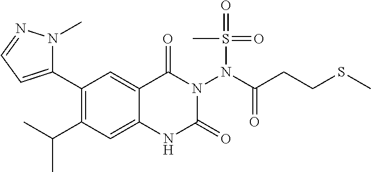 [SMILES: CSCCC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and 3-methylthiopropionyl chloride to yield N-[7-isopropyl-6-(2-methyl-2H-pyrazol-3-yl)-2,4-dioxo-1,4-dihydro-2H-quinazolin-3-yl]-N-(3-methylsulfanyl-propionyl)-methanesulfonamide. LC-MS at 254 nm; [M+H] 480; Rt 4.728 min; LC-MS Method V

### EXAMPLE 3.19

### [7-Isopropyl-6-(2-methyl-2H-pyrazol-3-yl)-2,4-dioxo-1,4-dihydro-2H-quinazolin-3-yl]-methanesulfonyl-carbamic acid ethyl ester

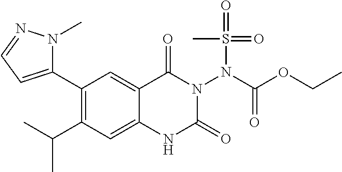 [SMILES: CCOC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and ethyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid ethyl ester. LC-MS at 254 nm; [M+H] 450; Rt 4.540 min; LC-MS Method V

### EXAMPLE 3.20

### [7-Isopropyl-6-(2-methyl-2H-pyrazol-3-yl)-2,4-dioxo-1,4-dihydro-2H-quinazolin-3-yl]-methanesulfonyl-carbamic acid propyl ester

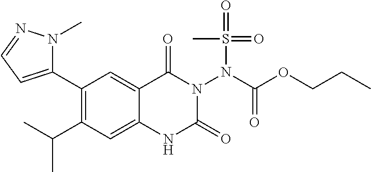 [SMILES: CCCOC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and propyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid propyl ester. LC-MS at 254 nm; [M+H] 464; Rt 4.835 min; LC-MS Method V

### EXAMPLE 3.21

### [7-Isopropyl-6-(2-methyl-2H-pyrazol-3-yl)-2,4-dioxo-1,4-dihydro-2H-quinazolin-3-yl]-methanesulfonyl-carbamic acid butyl ester

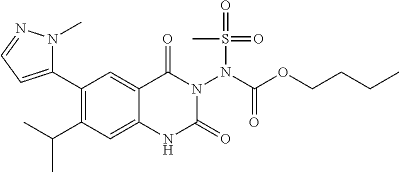 [SMILES: CCCCOC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and butyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid butyl ester. LC-MS at 254 nm; [M+H] 478; Rt 5.150 min; LC-MS Method V

### EXAMPLE 3.22

### [7-Isopropyl-6-(2-methyl-2H-pyrazol-3-yl)-2,4-dioxo-1,4-dihydro-2H-quinazolin-3-yl]-methanesulfonyl-carbamic acid hexyl ester

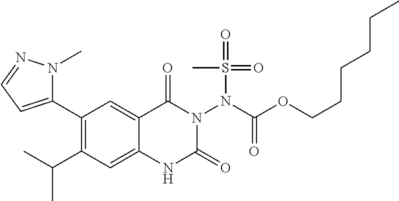 [SMILES: CCCCCCOC(=O)N(n1c(=O)[nH]c2cc(C(C)C)c(-c3ccnn3C)cc2c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and hexyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid hexyl ester. LC-MS at 254 nm; [M+H] 506; Rt 6.028 min; LC-MS Method V

### EXAMPLE 3.23

### N-(2,2-Dimethyl-propionyl)-N-[7-isopropyl-6-(2-methyl-2H-pyrazol-3-yl)-2,4-dioxo-1,4-dihydro-2H-quinazolin-3-yl]-methanesulfonamide

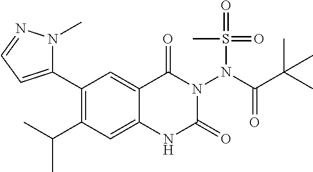 [SMILES: CC(C)c1cc2[nH]c(=O)n(N(C(=O)C(C)(C)C)S(C)(=O)=O)c(=O)c2cc1-c1ccnn1C]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and t-butyl chloroformate to yield N-(2,2-dimethyl-propionyl)-N-[7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 462; Rt 4.966 min; LC-MS Method V

### EXAMPLE 3.24

### N-Acetyl-N-[1-acetyl-7-isopropyl-6-(2-methyl-2H-pyrazol-3-yl)-2,4-dioxo-1,4-dihydro-2H-quinazolin-3-yl]-methanesulfonamide

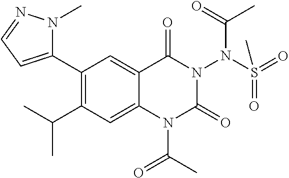 [SMILES: CC(=O)N(n1c(=O)c2cc(-c3ccnn3C)c(C(C)C)cc2n(C(C)=O)c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and acetyl chloride to yield N-acetyl-N-[1-acetyl-7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 462; Rt 5.296 min; LC-MS Method IV

### EXAMPLE 3.25

### N-[7-Isopropyl-6-(2-methyl-2H-pyrazol-3-yl)-2,4-dioxo-1-propionyl-1,4-dihydro-2H-quinazolin-3-yl]-N-propionyl-methanesulfonamide

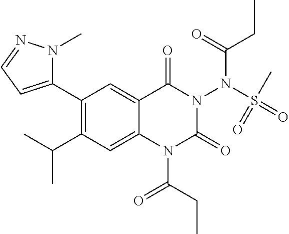 [SMILES: CCC(=O)N(n1c(=O)c2cc(-c3ccnn3C)c(C(C)C)cc2n(C(=O)CC)c1=O)S(C)(=O)=O]

Synthesis in analogy to Method D starting from N-[7-isopropyl-6-(2-methyl-2H-pyrazol-3-yl)-2,4-dioxo-1,4-dihydro-2H-quinazolin-3-yl]-methanesulfonamide and propionyl chloride to yield N-[7-isopropyl-6-(2-methyl-2H-pyrazol-3-yl)-2,4-dioxo-1-propionyl-1,4-dihydro-2H-quinazolin-3-yl]-N-propionyl-methanesulfonamide. LC-MS at 254 nm; [M+H] 490; Rt 5.589 min; LC-MS Method V

### EXAMPLE 3.26

### N-Butyryl-N-[1-butyryl-7-isopropyl-6-(2-methyl-2H-pyrazol-3-yl)-2,4-dioxo-1,4-dihydro-2H-quinazolin-3-yl]-methanesulfonamide

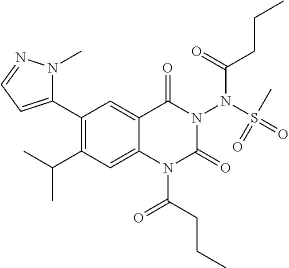 [SMILES: CCCC(=O)N(n1c(=O)c2cc(-c3ccnn3C)c(C(C)C)cc2n(C(=O)CCC)c1=O)S(C)(=O)=O]

Synthesis in analogy to Method D starting from N-[7-isopropyl-6-(2-methyl-2H-pyrazol-3-yl)-2,4-dioxo-1,4-dihydro-2H-quinazolin-3-yl]-methanesulfonamide and butyryl chloride to yield N-butyryl-N-[1-butyryl-7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 518; Rt 6.444 min; LC-MS Method V

### EXAMPLE 3.27

### [1-Acetyl-7-isopropyl-6-(2-methyl-2H-pyrazol-3-yl)-2,4-dioxo-1,4-dihydro-2H-quinazolin-3-yl]-methanesulfonyl-carbamic acid benzyl ester

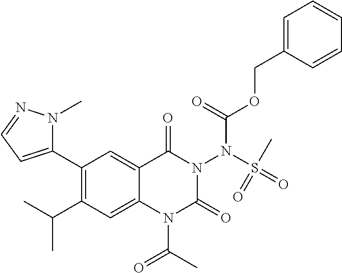 [SMILES: CC(=O)n1c(=O)n(N(C(=O)OCc2ccccc2)S(C)(=O)=O)c(=O)c2cc(-c3ccnn3C)c(C(C)C)cc21]

Synthesis in two steps. In the first step in analogy to Method B1 starting from N-[7-isopropyl-6-(2-methyl-2H-pyrazol-3-yl)-2,4-dioxo-1,4-dihydro-2H-quinazolin-3-yl]-methanesulfonamide and benzyl chloroformate to yield [7-isopropyl-6-(2-methyl-2H-pyrazol-3-yl)-2,4-dioxo-1,4-dihydro-2H-quinazolin-3-yl]-methanesulfonyl-carbamic acid benzyl ester LC-MS at 254 nm; [M+2H] 513; Rt 7.331 min (LC-MS Method I) and in a second step in analogy to Method D starting from [7-isopropyl-6-(2-methyl-2H-pyrazol-3-yl)-2,4-dioxo-1,4-dihydro-2H-quinazolin-3-yl]-methanesulfonyl-carbamic acid benzyl ester and acetyl chloride to yield [1-acetyl-7-isopropyl-6-(2-methyl-2H-pyrazol-3-yl)-2,4-dioxo-1,4-dihydro-2H-quinazolin-3-yl]-methanesulfonyl-carbamic acid benzyl ester. LC-MS at 254 nm; [M+H] 554; Rt 7.471 min; LC-MS Method V

### EXAMPLE 3.28

### N-[7-Isopropyl-6-(2-methyl-2H-pyrazol-3-yl)-2,4-dioxo-1,4-dihydro-2H-quinazolin-3-yl]-N-[2-[2-(2-methoxy-ethoxy)-ethoxy]-acetyl]-methanesulfonamide

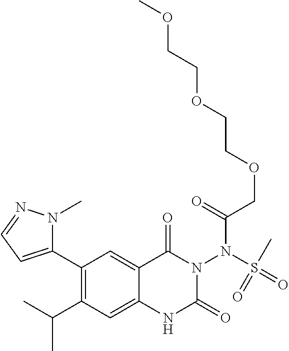 [SMILES: COCCOCCOCC(=O)N(n1c(=O)[nH]c2cc(C(C)C)c(-c3ccnn3C)cc2c1=O)S(C)(=O)=O]

Synthesis in analogy to Method D starting from N-[7-isopropyl-6-(2-methyl-2H-pyrazol-3-yl)-2,4-dioxo-1,4-dihydro-2H-quinazolin-3-yl]-methanesulfonamide and [2-(2-methoxy-ethoxy)-ethoxy]-acetyl chloride to yield N-[7-isopropyl-6-(2-methyl-2H-pyrazol-3-yl)-2,4-dioxo-1,4-dihydro-2H-quinazolin-3-yl]-N-{2-[2-(2-methoxy-ethoxy)-ethoxy]-acetyl}-methanesulfonamide. LC-MS at 254 nm; [M+H] 538; Rt 4.249 min; LC-MS Method V

### EXAMPLE 3.29

### N-(2-Benzyloxy-acetyl)-N-[7-isopropyl-6-(2-methyl-2H-pyrazol-3-yl)-2,4-dioxo-1,4-dihydro-2H-quinazolin-3-yl]-methanesulfonamide

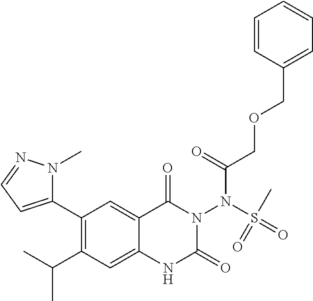 [SMILES: CC(C)c1cc2[nH]c(=O)n(N(C(=O)COCc3ccccc3)S(C)(=O)=O)c(=O)c2cc1-c1ccnn1C]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and benzyloxy-acetyl chloride to yield N-(2-benzyloxy-acetyl)-N-[7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 526; Rt 5.388 min; LC-MS Method V

### EXAMPLE 3.30

### N-(4-Benzyloxy-butyryl)-N-[7-isopropyl-6-(2-methyl-2H-pyrazol-3-yl)-2,4-dioxo-1,4-dihydro-2H-quinazolin-3-yl]-methanesulfonamide

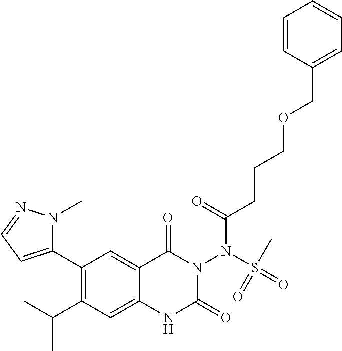 [SMILES: CC(C)c1cc2[nH]c(=O)n(N(C(=O)CCCOCc3ccccc3)S(C)(=O)=O)c(=O)c2cc1-c1ccnn1C]

Synthesis in analogy to Method F starting from N-[7-isopropyl-6-(2-methyl-2H-pyrazol-3-yl)-2,4-dioxo-1,4-dihydro-2H-quinazolin-3-yl]-methanesulfonamide and 4-benzyloxy-butyric acid to yield N-(4-benzyloxy-butyryl)-N-[7-isopropyl-6-(2-methyl-2H-pyrazol-3-yl)-2,4-dioxo-1,4-dihydro-2H-quinazolin-3-yl]-methanesulfonamide. LC-MS at 254 nm; [M+H] 554 Rt 5.331 min; LC-MS Method V

### EXAMPLE 3.31

### N-[7-Isopropyl-6-(2-methyl-2H-pyrazol-3-yl)-2,4-dioxo-1,4-dihydro-2H-quinazolin-3-yl]-N-(2-morpholin-4-yl-acetyl)-methanesulfonamide

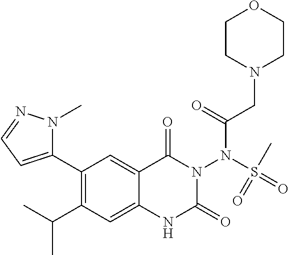 [SMILES: CC(C)c1cc2[nH]c(=O)n(N(C(=O)CN3CCOCC3)S(C)(=O)=O)c(=O)c2cc1-c1ccnn1C]

Synthesis in analogy to Method F starting from N-[7-isopropyl-6-(2-methyl-2H-pyrazol-3-yl)-2,4-dioxo-1,4-dihydro-2H-quinazolin-3-yl]-methanesulfonamide and morpholin-4-yl-acetic acid to yield N-[7-isopropyl-6-(2-methyl-2H-pyrazol-3-yl)-2,4-dioxo-1,4-dihydro-2H-quinazolin-3-yl]-N-(2-morpholin-4-yl-acetyp-methanesulfonamide. LC-MS at 254 nm; [M+H] 505; Rt 3.886 min; LC-MS Method V

### EXAMPLE 3.32

### 7-Isopropyl-3-(methoxycarbonyl-methanesulfonyl-amino)-6-(2-methyl-2H-pyrazol-3-yl)-2,4-dioxo-3,4-dihydro-2H-quinazoline-1-carboxylic acid methyl ester

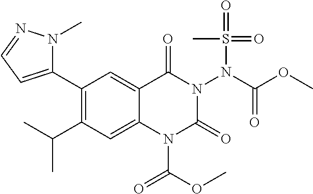 [SMILES: COC(=O)N(n1c(=O)c2cc(-c3ccnn3C)c(C(C)C)cc2n(C(=O)OC)c1=O)S(C)(=O)=O]

Synthesis in analogy to Method B1 starting from N-[7-isopropyl-6-(2-methyl-2H-pyrazol-3-yl)-2,4-dioxo-1,4-dihydro-2H-quinazolin-3-yl]-methanesulfonamide and methyl chloroformate to yield 7-isopropyl-3-(methoxycarbonyl-methanesulfonyl-amino)-6-(2-methyl-2H-pyrazol-3-yl)-2,4-dioxo-3,4-dihydro-2H-quinazoline-1-carboxylic acid methyl ester. LC-MS at 254 nm; [M+H] 494; Rt 6.563 min; LC-MS Method I

### EXAMPLE 3.33

### 3-(Ethoxycarbonyl-methanesulfonyl-amino)-7-isopropyl-6-(2-methyl-2H-pyrazol-3-y0-2,4-dioxo-3,4-dihydro-2H-quinazoline-1-carboxylic acid ethyl ester

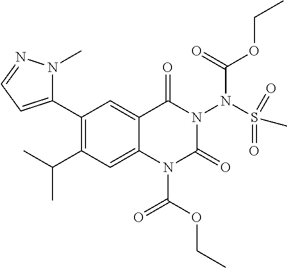 [SMILES: CCOC(=O)N(n1c(=O)c2cc(-c3ccnn3C)c(C(C)C)cc2n(C(=O)OCC)c1=O)S(C)(=O)=O]

Synthesis in analogy to Method B2.1 starting from N-[7-isopropyl-6-(2-methyl-2H-pyrazol-3-yl)-2,4-dioxo-1,4-dihydro-2H-quinazolin-3-yl]-methanesulfonamide and ethyl chloroformate to yield 3-(ethoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid ethyl ester. LC-MS at 254 nm; [M+H] 522; Rt 5.861 min; LC-MS Method V.

### EXAMPLE 3.34

### 7-Isopropyl-3-(methanesulfonyl-propoxycarbonyl-amino)-6-(2-methyl-2H-pyrazol-3-yl)-2,4-dioxo-3,4-dihydro-2H-quinazoline-1-carboxylic acid propyl ester

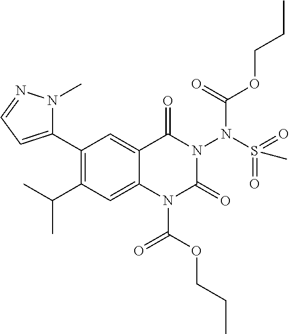 [SMILES: CCCOC(=O)N(n1c(=O)c2cc(-c3ccnn3C)c(C(C)C)cc2n(C(=O)OCCC)c1=O)S(C)(=O)=O]

Synthesis in analogy to Method B2.1 starting from N-[7-isopropyl-6-(2-methyl-2H-pyrazol-3-yl)-2,4-dioxo-1,4-dihydro-2H-quinazolin-3-yl]-methanesulfonamide and propyl chloroformate to yield 7-isopropyl-3-(methanesulfonyl-propoxycarbonyl-amino)-6-(2-methyl-2H-pyrazol-3-yl)-2,4-dioxo-3,4-dihydro-2H-quinazoline-1-carboxylic acid propyl ester. LC-MS at 254 nm; [M+H] 550; Rt 6.118 min; LC-MS Method V

### EXAMPLE 3.35

### 3-(Butoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid butyl ester

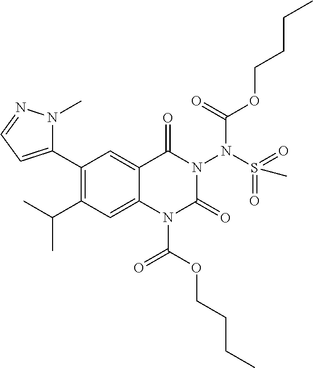 [SMILES: CCCCOC(=O)N(n1c(=O)c2cc(-c3ccnn3C)c(C(C)C)cc2n(C(=O)OCCCC)c1=O)S(C)(=O)=O]

Synthesis in analogy to Method A1 starting from N-[7-isopropyl-6-(2-methyl-2H-pyrazol-3-yl)-2,4-dioxo-1,4-dihydro-2H-quinazolin-3-yl]-methanesulfonamide and butyl chloroformate to yield 3-(butoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid butyl ester. LC-MS at 254 nm; [M+H] 578; Rt 6.938 min; LC-MS Method V

### EXAMPLE 3.36

### 3-(Acetyl-methanesulfonyl-amino)-7-isopropyl-6-(2-methyl-2H-pyrazol-3-yl)-2,4-dioxo-3,4-dihydro-2H-quinazoline-1-carboxylic acid methyl ester

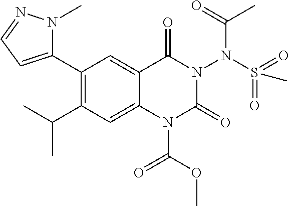 [SMILES: COC(=O)n1c(=O)n(N(C(C)=O)S(C)(=O)=O)c(=O)c2cc(-c3ccnn3C)c(C(C)C)cc21]

Synthesis in analogy to Method B2.2 starting from N-acetyl-N-[7-isopropyl-6-(2-methyl-2H-pyrazol-3-yl)-2,4-dioxo-1,4-dihydro-2H-quinazolin-3-yl]-methanesulfonamide (Example 3.01) and methyl chloroformate to yield 3-(acetyl-methanesulfonyl-amino)-7-isopropyl-6-(2-methyl-2H-pyrazol-3-yl)-2,4-dioxo-3,4-dihydro-2H-quinazoline-1-carboxylic acid methyl ester. LC-MS at 254 nm; [M+H] 478; Rt 5.213 min; LC-MS Method V

### EXAMPLE 3.37

### N-[7-Isopropyl-6-(2-methyl-2H-pyrazol-3-yl)-2,4-dioxo-1,4-dihydro-2H-quinazolin-3-yl]-N-(4-morpholin-4-yl-butyryl)-methanesulfonamide

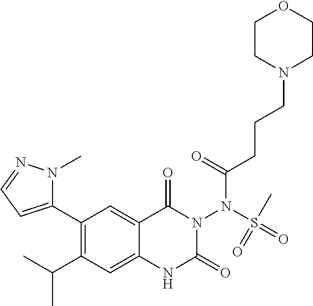 [SMILES: CC(C)c1cc2[nH]c(=O)n(N(C(=O)CCCN3CCOCC3)S(C)(=O)=O)c(=O)c2cc1-c1ccnn1C]

Synthesis in analogy to Method F starting from N-[7-isopropyl-6-(2-methyl-2H-pyrazol-3-yl)-2,4-dioxo-1,4-dihydro-2H-quinazolin-3-yl]-methanesulfonamide and 4-morpholin-4-yl-butyric acid to yield N-[7-isopropyl-6-(2-methyl-2H-pyrazol-3-yl)-2,4-dioxo-1,4-dihydro-2H-quinazolin-3-yl]-N-(4-morpholin-4-yl-butyryl)-methanesulfonamide. LC-MS at 254 nm; [M+H] 533; Rt 3.949 min; LC-MS Method V

### EXAMPLE 3.38

### N-[7-Isopropyl-6-(2-methyl-2H-pyrazol-3-yl)-2,4-dioxo-1,4-dihydro-2H-quinazolin-3-yl]-N-(3-morpholin-4-yl-propionyl)-methanesulfonamide

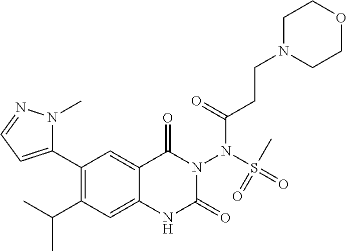 [SMILES: CC(C)c1cc2[nH]c(=O)n(N(C(=O)CCN3CCOCC3)S(C)(=O)=O)c(=O)c2cc1-c1ccnn1C]

Synthesis in analogy to Method F starting from N-[7-isopropyl-6-(2-methyl-2H-pyrazol-3-yl)-2,4-dioxo-1,4-dihydro-2H-quinazolin-3-yl]-methanesulfonamide and 3-morpholin-4-yl-propionic acid to yield N-[7-isopropyl-6-(2-methyl-2H-pyrazol-3-yl)-2,4-dioxo-1,4-dihydro-2H-quinazolin-3-yl]-N-(3-morpholin-4-yl-propionyl)-methanesulfonamide. LC-MS at 254 nm; [M+H] 519; Rt 3.932 min; LC-MS Method IV

### EXAMPLE 4.01

### 4-[(6-Imidazol-1-yl-2,4-dioxo-7-trifluoromethyl-1,4-dihydro-2H-quinazolin-3-yl)-methanesulfonyl-amino]-4-oxo-butyric acid ethyl ester

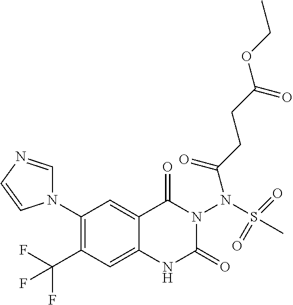 [SMILES: CCOC(=O)CCC(=O)N(n1c(=O)[nH]c2cc(C(F)(F)F)c(-n3ccnc3)cc2c1=O)S(C)(=O)=O]

N-(6-imidazol-1-yl-2,4-dioxo-7-trifluoromethyl-1,4-dihydro-2H-quinazolin-3-yl)-methanesulfonamide (584.0 mg, 1.5 mmol) is suspended in diisopropyl-ethylamine (0.321 mL; 1.875 mmol) and dry dichloromethane (15 mL) at room temperature (22° C.). Sequentially 3-chlorocarbonyl-propionic acid ethyl ester (272.0 mg, 1.650 mmol) is added and the reaction mixture is stirred at room temperature. After 1.5 hours the crude reaction mixture is poured on water and extracted three times with dichloromethane. The organic layer was dried over sodiumsulfate and evaporated. The crude product was purified by flash chromatography (ISCO CombiFlash; 40 g silica gel cartridge; dichloromethane/ethanol gradient: ethanol 0% to 10%) and the solvents evaporated to yield 4-[(6-imidazol-1-yl-2,4-dioxo-7-trifluoromethyl-1,4-dihydro-2H-quinazolin-3-yl)-methanesulfonyl-amino]-4-oxo-butyric acid ethyl ester. LC-MS at 254 nm; [M+H] 518; Rt 5.465 min; LC-MS Method III; ¹H-NMR (600 MHz; DMSO-d⁶) δ ppm 1.17 (t, 3H), 2.52 (t, 2H), 2.71 (m, 2H), 3.57 (s, 3H), 4.02 (q, 2H), 7.10 (d, J=0.5 Hz, 1H), 7.41 (d, J=0.5 Hz, 1H), 7.74 (s, 1H), 7.85 (s, 1H), 8.09 (s, 1H), 12.67 (s, 1H).

### EXAMPLE 4.02

### N-(6-Imidazol-1-yl-2,4-dioxo-7-trifluoromethyl-1,4-dihydro-2H-quinazolin-3-yl)-N-pentanoyl-methanesulfonamide

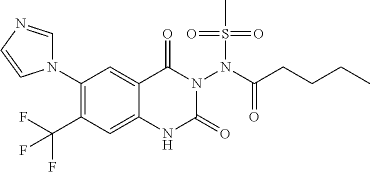 [SMILES: CCCCC(=O)N(n1c(=O)[nH]c2cc(C(F)(F)F)c(-n3ccnc3)cc2c1=O)S(C)(=O)=O]

Synthesis in analogy to Method E2 starting from N-(6-imidazol-1-yl-2,4-dioxo-7-trifluoromethyl-1,4-dihydro-2H-quinazolin-3-yl)-methanesulfonamide and pentanoyl chloride to yield N-(6-imidazol-1-yl-2,4-dioxo-7-trifluoromethyl-1,4-dihydro-2H-quinazolin-3-yl)-N-pentanoyl-methanesulfonamide. LC-MS at 254 nm; [M+H] 474; Rt 2.920 min; LC-MS Method II

### EXAMPLE 4.03

### N-Acetyl-N-(6-imidazol-1-yl-2,4-dioxo-7-trifluoromethyl-1,4-dihydro-2H-quinazolin-3-yl)-methanesulfonamide

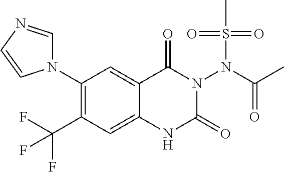 [SMILES: CC(=O)N(n1c(=O)[nH]c2cc(C(F)(F)F)c(-n3ccnc3)cc2c1=O)S(C)(=O)=O]

Synthesis in analogy to Method E2 starting from N-(6-imidazol-1-yl-2,4-dioxo-7-trifluoromethyl-1,4-dihydro-2H-quinazolin-3-yl)-methanesulfonamide and acetyl chloride to yield N-acetyl-N-(6-imidazol-1-yl-2,4-dioxo-7-trifluoromethyl-1,4-dihydro-2H-quinazolin-3-yl)-methanesulfonamide. LC-MS at 254 nm; [M+H] 432; Rt 2.015 min; LC-MS Method II

### EXAMPLE 4.04

### N-(6-Imidazol-1-yl-2,4-dioxo-7-trifluoromethyl-1,4-dihydro-2H-quinazolin-3-yl)-N-propionyl-methanesulfonamide

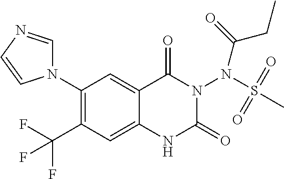 [SMILES: CCC(=O)N(n1c(=O)[nH]c2cc(C(F)(F)F)c(-n3ccnc3)cc2c1=O)S(C)(=O)=O]

Synthesis in analogy to Method E1 starting from N-(6-imidazol-1-yl-2,4-dioxo-7-trifluoromethyl-1,4-dihydro-2H-quinazolin-3-yl)-methanesulfonamide and propionyl chloride to yield N-(6-imidazol-1-yl-2,4-dioxo-7-trifluoromethyl-1,4-dihydro-2H-quinazolin-3-yl)-N-propionyl-methanesulfonamide. LC-MS at 254 nm; [M+H] 446; Rt 1.167 min; LC-MS Method I

### EXAMPLE 4.05

### N-(6-Imidazol-1-yl-2,4-dioxo-7-trifluoromethyl-1,4-dihydro-2H-quinazolin-3-yl)-N-isobutyryl-methanesulfonamide

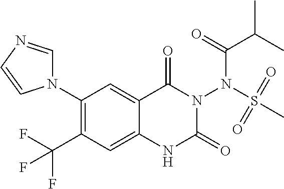 [SMILES: CC(C)C(=O)N(n1c(=O)[nH]c2cc(C(F)(F)F)c(-n3ccnc3)cc2c1=O)S(C)(=O)=O]

Synthesis in analogy to Method E1 starting from N-(6-imidazol-1-yl-2,4-dioxo-7-trifluoromethyl-1,4-dihydro-2H-quinazolin-3-yl)-methanesulfonamide and isobutyryl chloride to yield N-(6-imidazol-1-yl-2,4-dioxo-7-trifluoromethyl-1,4-dihydro-2H-quinazolin-3-yl)-N-isobutyryl-methanesulfonamide. LC-MS at 254 nm; [M+H] 460; Rt 1.054 min; LC-MS Method I

### EXAMPLE 4.06

### N-(6-Imidazol-1-yl-2,4-dioxo-7-trifluoromethyl-1,4-dihydro-2H-quinazolin-3-yl)-N-(3-methyl-butyryl)-methanesulfonamide

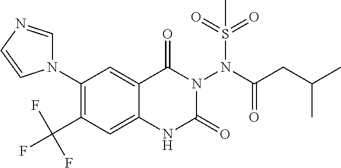 [SMILES: CC(C)CC(=O)N(n1c(=O)[nH]c2cc(C(F)(F)F)c(-n3ccnc3)cc2c1=O)S(C)(=O)=O]

Synthesis in analogy to Method E2 starting from N-(6-imidazol-1-yl-2,4-dioxo-7-trifluoromethyl-1,4-dihydro-2H-quinazolin-3-yl)-methanesulfonamide and 3-methyl-butyryl chloride to yield N-(6-imidazol-1-yl-2,4-dioxo-7-trifluoromethyl-1,4-dihydro-2H-quinazolin-3-yl)-N-(3-methyl-butyryl)-methanesulfonamide. LC-MS at 254 nm; [M+H] 474; Rt 1.140 min; LC-MS Method I

### EXAMPLE 4.07

### N-Cyclopentanecarbonyl-N-(6-imidazol-1-yl-2,4-dioxo-7-trifluoromethyl-1,4-dihydro-2H-quinazolin-3-yp-methanesulfonamide

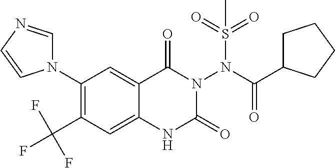 [SMILES: CS(=O)(=O)N(C(=O)C1CCCC1)n1c(=O)[nH]c2cc(C(F)(F)F)c(-n3ccnc3)cc2c1=O]

Synthesis in analogy to Method E2 starting from N-(6-imidazol-1-yl-2,4-dioxo-7-trifluoromethyl-1,4-dihydro-2H-quinazolin-3-yl)-methanesulfonamide and cyclopentanecarbonyl chloride to yield N-cyclopentanecarbonyl-N-(6-imidazol-1-yl-2,4-dioxo-7-trifluoromethyl-1,4-dihydro-2H-quinazolin-3-yl)-methanesulfonamide. LC-MS at 254 nm; [M+H] 486; Rt 5.198 min; LC-MS Method I

### EXAMPLE 4.08

### N-Hexanoyl-N-(6-imidazol-1-yl-2,4-dioxo-7-trifluoromethyl-1,4-dihydro-2H-quinazolin-3-yl)-methanesulfonamide

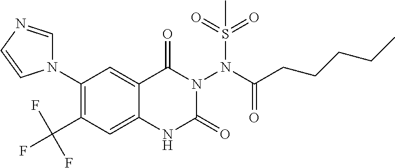 [SMILES: CCCCCC(=O)N(n1c(=O)[nH]c2cc(C(F)(F)F)c(-n3ccnc3)cc2c1=O)S(C)(=O)=O]

Synthesis in analogy to Method E2 starting from N-(6-imidazol-1-yl-2,4-dioxo-7-trifluoromethyl-1,4-dihydro-2H-quinazolin-3-yl)-methanesulfonamide and hexanoyl chloride to yield N-hexanoyl-N-(6-imidazol-1-yl-2,4-dioxo-7-trifluoromethyl-1,4-dihydro-2H-quinazolin-3-yl)-methanesulfonamide. LC-MS at 254 nm; [M+H] 488; Rt 5.620 min; LC-MS Method I

### EXAMPLE 4.09

### N-Butyryl-N-(6-imidazol-1-yl-2,4-dioxo-7-trifluoromethyl-1,4-dihydro-2H-quinazolin-3-yl)-methanesulfonamide

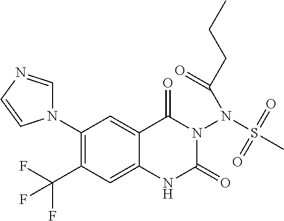 [SMILES: CCCC(=O)N(n1c(=O)[nH]c2cc(C(F)(F)F)c(-n3ccnc3)cc2c1=O)S(C)(=O)=O]

Synthesis in analogy to Method E1 starting from N-(6-imidazol-1-yl-2,4-dioxo-7-trifluoromethyl-1,4-dihydro-2H-quinazolin-3-yl)-methanesulfonamide and butyryl chloride to yield N-butyryl-N-(6-imidazol-1-yl-2,4-dioxo-7-trifluoromethyl-1,4-dihydro-2H-quinazolin-3-yl)-methanesulfonamide. LC-MS at 254 nm; [M+H] 460; Rt 1.297 min; LC-MS Method I

### EXAMPLE 5.01

### N-Acetyl-N-[6-(2-isopropyl-2H-pyrazol-3-yl)-2,4-dioxo-7-trifluoromethyl-1,4-dihydro-2H-quinazolin-3-yl]-methanesulfonamide

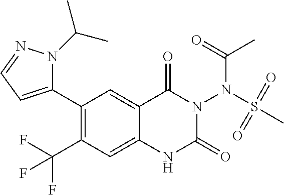 [SMILES: CC(=O)N(n1c(=O)[nH]c2cc(C(F)(F)F)c(-c3ccnn3C(C)C)cc2c1=O)S(C)(=O)=O]

N-[6-(2-isopropyl-2H-pyrazol-3-yl)-2,4-dioxo-7-trifluoromethyl-1,4-dihydro-2H-quinazolin-3-yl]-methanesulfonamide (108.0 mg, 0.250 mmol) is dissolved in dry dichloromethane (1 mL) and pyridine (24.72 mg, 0.313 mmol) at room temperature (22° C.). Sequentially the acetyl chloride (21.59 mg, 0.275 mmol) is added and the reaction mixture is stirred at room temperature for 1.5 hour. Subsequently the solvent is evaporated and the crude product is purified by silica gel flash chromatography (ISCO CombiFlash; 24 g silica gel cartridge; cyclohexane/ethyl acetate gradient: ethyl acetate 0% to 75%) to yield N-acetyl-N-[6-(2-isopropyl-2H-pyrazol-3-yl)-2,4-dioxo-7-trifluoromethyl-1,4-dihydro-2H-quinazolin-3-yl]-methanesulfonamide. LC-MS at 254 nm; [M+H] 474; Rt 5.123 min, LC-MS Method IV; ¹H-NMR (600 MHz; DMSO-d⁶) δ ppm 1.30 (broad d, 6H), 2.18 (broad s, 3H), 3.59 (s, 3H), 4.12 (m, 1H), 6.29 (broad d, 1H), 7.58 (d, J=0.5 Hz, 1H), 7.77 (s, 1H), 7.98 (s, 1H), 12.60 (s, 1H).

### EXAMPLE 5.02

### N-[6-(2-Isopropyl-2H-pyrazol-3-yl)-2,4-dioxo-7-trifluoromethyl-1,4-dihydro-2H-quinazolin-3-yl]-N-propionyl-methanesulfonamide

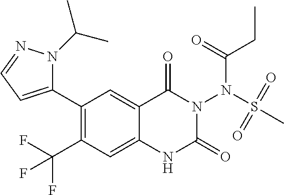 [SMILES: CCC(=O)N(n1c(=O)[nH]c2cc(C(F)(F)F)c(-c3ccnn3C(C)C)cc2c1=O)S(C)(=O)=O]

Synthesis in analogy to Method C2 starting from N-[6-(2-isopropyl-2H-pyrazol-3-yl)-2,4-dioxo-7-trifluoromethyl-1,4-dihydro-2H-quinazolin-3-yl]-methanesulfonamide and propionyl chloride to yield N-[6-(2-isopropyl-2H-pyrazol-3-yl)-2,4-dioxo-7-trifluoromethyl-1,4-dihydro-2H-quinazolin-3-yl]-N-propionyl-methanesulfonamide. LC-MS at 254 nm; [M+H] 488; Rt 5.591 min; LC-MS Method IV

### EXAMPLE 5.03

### N-Butyryl-N-[6-(2-isopropyl-2H-pyrazol-3-yl)-2,4-dioxo-7-trifluoromethyl-1,4-dihydro-2H-quinazolin-3-yl]-methanesulfonamide

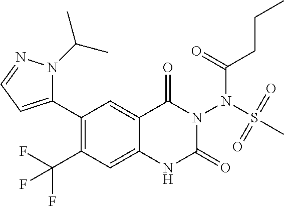 [SMILES: CCCC(=O)N(n1c(=O)[nH]c2cc(C(F)(F)F)c(-c3ccnn3C(C)C)cc2c1=O)S(C)(=O)=O]

Synthesis in analogy to Method C2 starting from N-[6-(2-isopropyl-2H-pyrazol-3-yl)-2,4-dioxo-7-trifluoromethyl-1,4-dihydro-2H-quinazolin-3-yl]-methanesulfonamide and butyryl chloride to yield N-butyryl-N-[6-(2-isopropyl-2H-pyrazol-3-yl)-2,4-dioxo-7-trifluoromethyl-1,4-dihydro-2H-quinazolin-3-yl]-methanesulfonamide. LC-MS at 254 nm; [M+H] 502; Rt 5.914 min; LC-MS Method IV

### EXAMPLE 5.04

### N-Hexanoyl-N-[6-(2-isopropyl-2H-pyrazol-3-yl)-2,4-dioxo-7-trifluoromethyl-1,4-dihydro-2H-quinazolin-3-yl]-methanesulfonamide

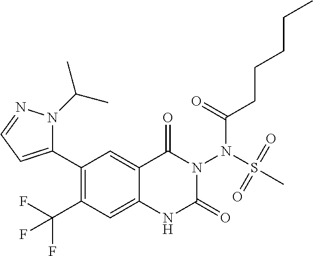 [SMILES: CCCCCC(=O)N(n1c(=O)[nH]c2cc(C(F)(F)F)c(-c3ccnn3C(C)C)cc2c1=O)S(C)(=O)=O]

Synthesis in analogy to Method C2 starting from N-[6-(2-isopropyl-2H-pyrazol-3-yl)-2,4-dioxo-7-trifluoromethyl-1,4-dihydro-2H-quinazolin-3-yl]-methanesulfonamide and hexanoyl chloride to yield N-hexanoyl-N-[6-(2-isopropyl-2H-pyrazol-3-yl)-2,4-dioxo-7-trifluoromethyl-1,4-dihydro-2H-quinazolin-3-yl]-methanesulfonamide. LC-MS at 254 nm; [M+H] 530; Rt 6.575 min; LC-MS Method IV

### EXAMPLE 5.05

### N-Isobutyryl-N-[6-(2-isopropyl-2H-pyrazol-3-yl)-2,4-dioxo-7-trifluoromethyl-1,4-dihydro-2H-quinazolin-3-yl]-methanesulfonamide

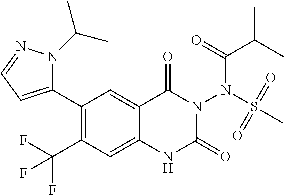 [SMILES: CC(C)C(=O)N(n1c(=O)[nH]c2cc(C(F)(F)F)c(-c3ccnn3C(C)C)cc2c1=O)S(C)(=O)=O]

Synthesis in analogy to Method C2 starting from N-[6-(2-isopropyl-2H-pyrazol-3-yl)-2,4-dioxo-7-trifluoromethyl-1,4-dihydro-2H-quinazolin-3-yl]-methanesulfonamide and isobutyryl chloride to yield N-isobutyryl-N-[6-(2-isopropyl-2H-pyrazol-3-yl)-2,4-dioxo-7-trifluoromethyl-1,4-dihydro-2H-quinazolin-3-yl]-methanesulfonamide. LC-MS at 254 nm; [M+H] 502; Rt 6.145 min; LC-MS Method IV

### EXAMPLE 5.06

### [6-(2-Isopropyl-2H-pyrazol-3-yl)-2,4-dioxo-7-trifluoromethyl-1,4-dihydro-2H-quinazolin-3-yl]-methanesulfonyl-carbamic acid ethyl ester

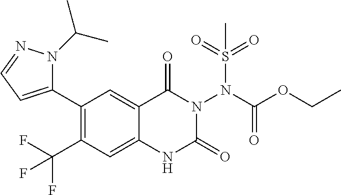 [SMILES: CCOC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and ethyl chloroformate to yield [6-(2-isopropyl-2H-pyrazol-3-yl)-2,4-dioxo-7-trifluoromethyl-1,4-dihydro-2H-quinazolin-3-yl]-methanesulfonyl-carbamic acid ethyl ester. LC-MS at 216 nm; [M+H] 504; Rt 5.653 min; LC-MS Method III

### EXAMPLE 5.07

### [6-(2-Isopropyl-2H-pyrazol-3-yl)-2,4-dioxo-7-trifluoromethyl-1,4-dihydro-2H-quinazolin-3-yl]-methanesulfonyl-carbamic acid propyl ester

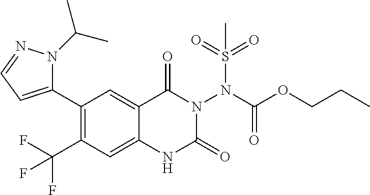 [SMILES: CCCOC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and propyl chloroformate to yield [6-(2-isopropyl-2H-pyrazol-3-yl)-2,4-dioxo-7-trifluoromethyl-1,4-dihydro-2H-quinazolin-3-yl]-methanesulfonyl-carbamic acid propyl ester. LC-MS at 216 nm; [M+H] 518; Rt 6.274 min; LC-MS Method III

### EXAMPLE 5.08

### [6-(2-Isopropyl-2H-pyrazol-3-yl)-2,4-dioxo-7-trifluoromethyl-1,4-dihydro-2H-quinazolin-3-yl]-methanesulfonyl-carbamic acid butyl ester

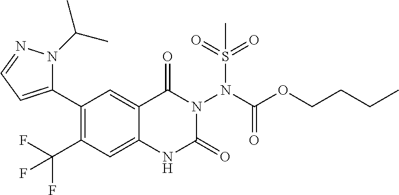 [SMILES: CCCCOC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and butyl chloroformate to yield [6-(2-isopropyl-2H-pyrazol-3-yl)-2,4-dioxo-7-trifluoromethyl-1,4-dihydro-2H-quinazolin-3-yl]-methanesulfonyl-carbamic acid butyl ester. LC-MS at 216 nm; [M+H] 532; Rt 6.236 min; LC-MS Method IV

### EXAMPLE 5.09

### [6-(2-Isopropyl-2H-pyrazol-3-yl)-2,4-dioxo-7-trifluoromethyl-1,4-dihydro-2H-quinazolin-3-yl]-methanesulfonyl-carbamic acid isobutyl ester

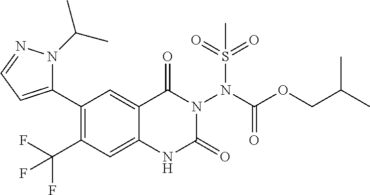 [SMILES: CC(C)COC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and isobutyl chloroformate to yield [6-(2-isopropyl-2H-pyrazol-3-yl)-2,4-dioxo-7-trifluoromethyl-1,4-dihydro-2H-quinazolin-3-yl]-methanesulfonyl-carbamic acid isobutyl ester. LC-MS at 216 nm; [M+H] 532; Rt 6.260 min; LC-MS Method IV

### EXAMPLE 5.10

### [6-(2-Isopropyl-2H-pyrazol-3-yl)-2,4-dioxo-7-trifluoromethyl-1,4-dihydro-2H-quinazolin-3-yl]-methanesulfonyl-carbamic acid 2-methoxy-ethyl ester

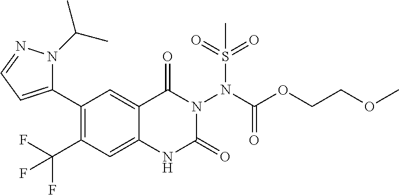 [SMILES: COCCOC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and 2-methoxyethyl chloroformate to yield [6-(2-isopropyl-2H-pyrazol-3-yl)-2,4-dioxo-7-trifluoromethyl-1,4-dihydro-2H-quinazolin-3-yl]-methanesulfonyl-carbamic acid 2-methoxy-ethyl ester. LC-MS at 254 nm; [M+H] 534; Rt 5.751 min; LC-MS Method IV

### EXAMPLE 5.11

### 5-{[6-(2-Isopropyl-2H-pyrazol-3-yl)-2,4-dioxo-7-trifluoromethyl-1,4-dihydro-2H-quinazolin-3-yl]-methanesulfonyl-amino}-5-oxo-pentanoic acid ethyl ester

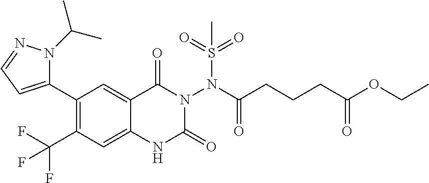 [SMILES: CCOC(=O)CCCC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and ethyl glutaryl chloride to yield 5-{[6-(2-isopropyl-2H-pyrazol-3-yl)-2,4-dioxo-7-trifluoromethyl-1,4-dihydro-2H-quinazolin-3-yl]-methanesulfonyl-amino}-5-oxo-pentanoic acid ethyl ester. LC-MS at 216 nm; [M+H] 574; Rt 5.877 min; LC-MS Method IV

### EXAMPLE 5.12

### 3-{[6-(2-Isopropyl-2H-pyrazol-3-yl)-2,4-dioxo-7-trifluoromethyl-1,4-dihydro-2H-quinazolin-3-yl]-methanesulfonyl-amino}-3-oxo-propionic acid ethyl ester

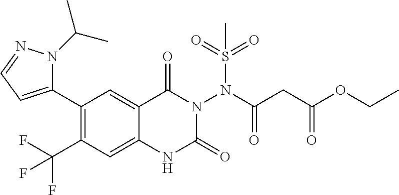 [SMILES: CCOC(=O)CC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and ethyl malonyl chloride to yield 3-{[6-(2-isopropyl-2H-pyrazol-3-yl)-2,4-dioxo-7-trifluoromethyl-1,4-dihydro-2H-quinazolin-3-yl]-methanesulfonyl-amino}-3-oxo-propionic acid ethyl ester. LC-MS at 216 nm; [M+H] 546; Rt 5.964 min; LC-MS Method IV

### EXAMPLE 5.13

### N-[6-(2-Isopropyl-2H-pyrazol-3-yl)-2,4-dioxo-7-trifluoromethyl-1,4-dihydro-2H-quinazolin-3-yl]-N-(3-methylsulfanyl-propionyl)-methanesulfonamide

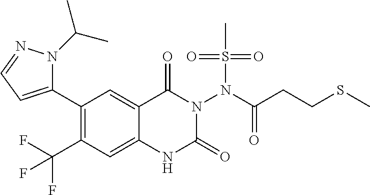 [SMILES: CSCCC(=O)N(n1c(=O)[nH]c2cc(C(F)(F)F)c(-c3ccnn3C(C)C)cc2c1=O)S(C)(=O)=O]

Synthesis in analogy to Method A2 starting from N-[6-(2-isopropyl-2H-pyrazol-3-yl)-2,4-dioxo-7-trifluoromethyl-1,4-dihydro-2H-quinazolin-3-yl]-methanesulfonamide and 3-methylthiopropionyl chloride to yield N-[6-(2-isopropyl-2H-pyrazol-3-yl)-2,4-dioxo-7-trifluoromethyl-1,4-dihydro-2H-quinazolin-3-yl]-N-(3-methylsulfanyl-propionyl)-methanesulfonamide. LC-MS at 216 nm; [M+H] 534; Rt 5.998 min; LC-MS Method IV

### EXAMPLE 6.01

### N-Acetyl-N-[7-difluoromethyl-6-(2-isopropyl-2H-pyrazol-3-yl)-2,4-dioxo-1,4-dihydro-2H-quinazolin-3-yl]-methanesulfonamide

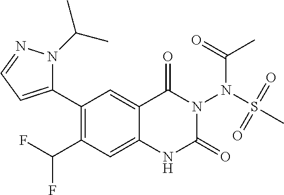 [SMILES: CC(=O)N(n1c(=O)[nH]c2cc(C(F)F)c(-c3ccnn3C(C)C)cc2c1=O)S(C)(=O)=O]

N-[7-difluoromethyl-6-(2-isopropyl-2H-pyrazol-3-yl)-2,4-dioxo-1,4-dihydro-2H-quinazolin-3-yl]-methanesulfonamide (90.0 mg, 0.218 mmol) is dissolved in pyridine (0.871 mL) at room temperature (22° C.). Sequentially acetyl chloride (18.8 mg, 0.239 mmol) is added and the reaction mixture is stirred at room temperature for 1 hour. Subsequently the solvent is evaporated and the crude product is purified by silica gel flash chromatography (ISCO CombiFlash; 12 g silica gel cartridge; methylene chloride/ethanol gradient: ethanol 0% to 5%) to yield N-acetyl-N-[7-difluoromethyl-6-(2-isopropyl-2H-pyrazol-3-yl)-2,4-dioxo-1,4-dihydro-2H-quinazolin-3-yl]-methanesulfonamide. LC-MS at 254 nm; [M+H] 456; Rt 5.003 min; LC-MS Method IV; ¹H-NMR (600 MHz; DMSO-d⁶) δ ppm 1.31 (broad d, 6H), 2.15 (broad s, 3H), 3.60 (s, 3H), 4.16 (m, 1H), 6.35 (d, J=0.5 Hz, 1H), 6.87 (t, 1H), 7.61 (d, J=0.5 Hz, 1H), 7.64 (s, 1H), 7.93 (s, 1H), 12.50 (s, 1H).

### EXAMPLE 6.02

### N-Butyryl-N-[7-difluoromethyl-6-(2-isopropyl-2H-pyrazol-3-yl)-2,4-dioxo-1,4-dihydro-2H-quinazolin-3-yl]-methanesulfonamide

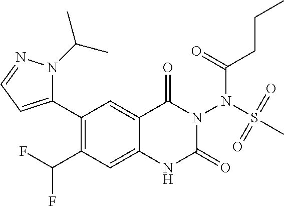 [SMILES: CCCC(=O)N(n1c(=O)[nH]c2cc(C(F)F)c(-c3ccnn3C(C)C)cc2c1=O)S(C)(=O)=O]

Synthesis in analogy to Method C1 starting from N-[7-difluoromethyl-6-(2-isopropyl-2H-pyrazol-3-yl)-2,4-dioxo-1,4-dihydro-2H-quinazolin-3-yl]-methanesulfonamide and butyryl chloride to yield N-butyryl-N-[7-difluoromethyl-6-(2-isopropyl-2H-pyrazol-3-yl)-2,4-dioxo-1,4-dihydro-2H-quinazolin-3-yl]-methanesulfonamide. LC-MS at 254 nm; [M+H] 484; Rt 5.693 min; LC-MS Method IV

### EXAMPLE 6.03

### N-[7-Difluoromethyl-6-(2-isopropyl-2H-pyrazol-3-yl)-2,4-dioxo-1,4-dihydro-2H-quinazolin-3-yl]-N-pentanoyl-methanesulfonamide

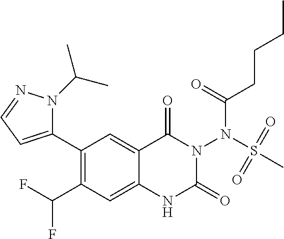 [SMILES: CCCCC(=O)N(n1c(=O)[nH]c2cc(C(F)F)c(-c3ccnn3C(C)C)cc2c1=O)S(C)(=O)=O]

Synthesis in analogy to Method C1 starting from N-[7-difluoromethyl-6-(2-isopropyl-2H-pyrazol-3-yl)-2,4-dioxo-1,4-dihydro-2H-quinazolin-3-yl]-methanesulfonamide and pentanoyl chloride to yield N-[7-difluoromethyl-6-(2-isopropyl-2H-pyrazol-3-yl)-2,4-dioxo-1,4-dihydro-2H-quinazolin-3-yl]-N-pentanoyl-methanesulfonamide. LC-MS at 254 nm; [M+H] 498; Rt 6.330 min.; LC-MS Method IV

### EXAMPLE 6.04

### [7-Difluoromethyl-6-(2-isopropyl-2H-pyrazol-3-yl)-2,4-dioxo-1,4-dihydro-2H-quinazolin-3-yl]-methanesulfonyl-carbamic acid propyl ester

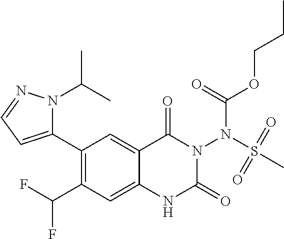 [SMILES: CCCOC(=O)N(n1c(=O)[nH]c2cc(C(F)F)c(-c3ccnn3C(C)C)cc2c1=O)S(C)(=O)=O]

Synthesis in analogy to Method C1 starting from N-[7-difluoromethyl-6-(2-isopropyl-2H-pyrazol-3-yl)-2,4-dioxo-1,4-dihydro-2H-quinazolin-3-yl]-methanesulfonamide and propyl chloroformate to yield [7-difluoromethyl-6-(2-isopropyl-2H-pyrazol-3-yl)-2,4-dioxo-1,4-dihydro-2H-quinazolin-3-yl]-methanesulfonyl-carbamic acid propyl ester. LC-MS at 254 nm; [M+H] 500; Rt 6.051 min; LC-MS Method IV

### EXAMPLE 6.05

### N-[7-Difluoromethyl-6-(2-isopropyl-2H-pyrazol-3-yl)-2,4-dioxo-1,4-dihydro-2H-quinazolin-3-yl]-N-propionyl-methanesulfonamide

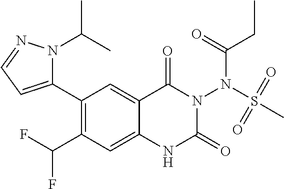 [SMILES: CCC(=O)N(n1c(=O)[nH]c2cc(C(F)F)c(-c3ccnn3C(C)C)cc2c1=O)S(C)(=O)=O]

Synthesis in analogy to Method A2 starting from N-[7-difluoromethyl-6-(2-isopropyl-2H-pyrazol-3-yl)-2,4-dioxo-1,4-dihydro-2H-quinazolin-3-yl]-methanesulfonamide and propionyl chloride to yield N-[7-difluoromethyl-6-(2-isopropyl-2H-pyrazol-3-yl)-2,4-dioxo-1,4-dihydro-2H-quinazolin-3-yl]-N-propionyl-methanesulfonamide. LC-MS at 254 nm; [M+H] 470; Rt 5.241 min; LC-MS Method IV

### EXAMPLE 6.06

### [7-Difluoromethyl-6-(2-isopropyl-2H-pyrazol-3-yl)-2,4-dioxo-1,4-dihydro-2H-quinazolin-3-yl]-methanesulfonyl-carbamic acid ethyl ester

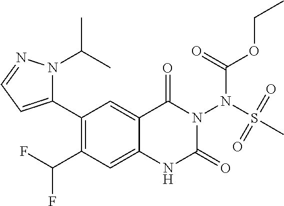 [SMILES: CCOC(=O)N(n1c(=O)[nH]c2cc(C(F)F)c(-c3ccnn3C(C)C)cc2c1=O)S(C)(=O)=O]

Synthesis in analogy to Method A2 starting from N-[7-difluoromethyl-6-(2-isopropyl-2H-pyrazol-3-yl)-2,4-dioxo-1,4-dihydro-2H-quinazolin-3-yl]-methanesulfonamide and ethyl chloroformate to yield [7-difluoromethyl-6-(2-isopropyl-2H-pyrazol-3-yl)-2,4-dioxo-1,4-dihydro-2H-quinazolin-3-yl]-methanesulfonyl-carbamic acid ethyl ester. LC-MS at 254 nm; [M+H] 486; Rt 5.647 min; LC-MS Method IV

### EXAMPLE 6.07

### [7-Difluoromethyl-6-(2-isopropyl-2H-pyrazol-3-yl)-2,4-dioxo-1,4-dihydro-2H-quinazolin-3-yl]-methanesulfonyl-carbamic acid 2-methoxy-ethyl ester

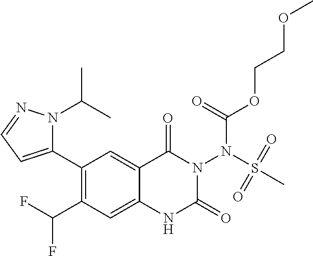 [SMILES: COCCOC(=O)N(n1c(=O)[nH]c2cc(C(F)F)c(-c3ccnn3C(C)C)cc2c1=O)S(C)(=O)=O]

Synthesis in analogy to Method A2 starting from N-[7-difluoromethyl-6-(2-isopropyl-2H-pyrazol-3-yl)-2,4-dioxo-1,4-dihydro-2H-quinazolin-3-yl]-methanesulfonamide and 2-methoxyethyl chloroformate to yield [7-difluoromethyl-6-(2-isopropyl-2H-pyrazol-3-yl)-2,4-dioxo-1,4-dihydro-2H-quinazolin-3-yl]-methanesulfonyl-carbamic acid 2-methoxy-ethyl ester. LC-MS at 254 nm; [M+H] 516; Rt 5.382 min; LC-MS Method IV

### EXAMPLE 7.01

### Methanesulfonyl-[6-(2-methyl-imidazol-1-yl)-2,4-dioxo-7-trifluoromethyl-1,4-dihydro-2H-quinazolin-3-yl]-carbamic acid ethyl ester

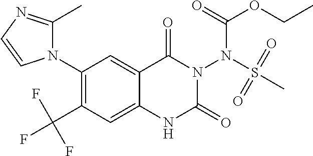 [SMILES: CCOC(=O)N(n1c(=O)[nH]c2cc(C(F)(F)F)c(-n3ccnc3C)cc2c1=O)S(C)(=O)=O]

N-[6-(2-Methyl-imidazol-1-yl)-2,4-dioxo-7-trifluoromethyl-1,4-dihydro-2H-quinazolin-3-yl]-methanesulfonamide (60 mg, 0.149 mmol) is dissolved in pyridine (1.5 ml) at room temperature (22° C.). Sequentially ethyl chloroformate (24.22 mg, 0.223 mmol) is added and the reaction mixture is stirred at room temperature for 1 hour. Subsequently the solvent is evaporated and the crude product is purified by silica gel flash chromatography (ISCO Companion Flash; 4 g silica gel cartridge; hexane/ethyl acetate gradient: ethyl acetate 0% to 100%). Then the solvent is evaporated to yield methanesulfonyl-[6-(2-methyl-imidazol-1-yl)-2,4-dioxo-7-trifluoromethyl-1,4-dihydro-2H-quinazolin-3-yl]-carbamic acid ethyl ester (34 mg, 0.070 mmol, 47% yield) as white resin. LC-MS at 254 nm; M+H 476; Rt 4.457 min (method: LC-MS Method I). ¹H-NMR (600 MHz; DMSO-d⁶) [symbol] ppm 1.27 (m, 3H), 2.06 (s, 3H), 3.67 (2s, 3H), 4.32 (m, 2H), 6.93 (d, J=1 Hz, 1H), 7.21 (d, J=1 Hz, 1H), 7.75 (s, 1H), 8.12 (2s, 1H).

### EXAMPLE 7.02

### N-Butyryl-N-[6-(2-methyl-imidazol-1-yl)-2,4-dioxo-7-trifluoromethyl-1,4-dihydro-2H-quinazolin-3-yl]-methanesulfonamide

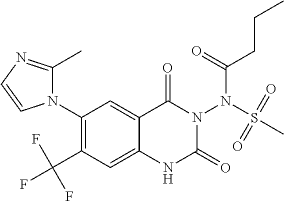 [SMILES: CCCC(=O)N(n1c(=O)[nH]c2cc(C(F)(F)F)c(-n3ccnc3C)cc2c1=O)S(C)(=O)=O]

Synthesis in analogy to Method C1 starting from N-[6-(2-methyl-imidazol-1-yl)-2,4-dioxo-7-trifluoromethyl-1,4-dihydro-2H-quinazolin-3-yl]-methanesulfonamide and butyryl chloride to yield N-butyryl-N-[6-(2-methyl-imidazol-1-yl)-2,4-dioxo-7-trifluoromethyl-1,4-dihydro-2H-quinazolin-3-yl]-methanesulfonamide. LC-MS at 254 nm; [M+H] 474; Rt 1.110 min; (LC-MS

Method I)

### EXAMPLE 7.03

### Methanesulfonyl-[6-(2-methyl-imidazol-1-yl)-2,4-dioxo-7-trifluoromethyl-1,4-dihydro-2H-quinazolin-3-yl]-carbamic acid propyl ester

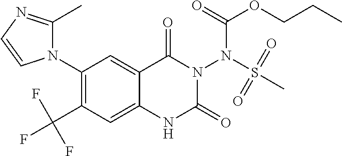 [SMILES: CCCOC(=O)N(n1c(=O)[nH]c2cc(C(F)(F)F)c(-n3ccnc3C)cc2c1=O)S(C)(=O)=O]

Synthesis in analogy to Method C1 starting from N-[6-(2-methyl-imidazol-1-yl)-2,4-dioxo-7-trifluoromethyl-1,4-dihydro-2H-quinazolin-3-yl]-methanesulfonamide and propyl chloroformate to yield methanesulfonyl-[6-(2-methyl-imidazol-1-yl)-2,4-dioxo-7-trifluoromethyl-1,4-dihydro-2H-quinazolin-3-yl]-carbamic acid propyl ester. LC-MS at 254 nm; [M+H] 490; Rt 4.828 min; (LC/MS-Method I)

### EXAMPLE 7.04

### Methanesulfonyl-[6-(2-methyl-imidazol-1-yl)-2,4-dioxo-7-trifluoromethyl-1,4-dihydro-2H-quinazolin-3-yl]-carbamic acid isobutyl ester

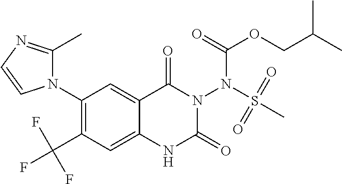 [SMILES: Cc1nccn1-c1cc2c(=O)n(N(C(=O)OCC(C)C)S(C)(=O)=O)c(=O)[nH]c2cc1C(F)(F)F]

Synthesis in analogy to Method C1 starting from N-[6-(2-methyl-imidazol-1-yl)-2,4-dioxo-7-trifluoromethyl-1,4-dihydro-2H-quinazolin-3-yl]-methanesulfonamide and isobutyl chloroformate to yield methanesulfonyl-[6-(2-methyl-imidazol-1-yl)-2,4-dioxo-7-trifluoromethyl-1,4-dihydro-2H-quinazolin-3-yl]-carbamic acid isobutyl ester. LC-MS at 254 nm; [M+H] 504; Rt 5.159 min; (LC-MS Method I)

### EXAMPLE 8.01

### N-(2,4-Dioxo-6-pyrrol-1-yl-7-trifluoromethyl-1,4-dihydro-2H-quinazolin-3-yl)-N-propionyl-methanesulfonamide

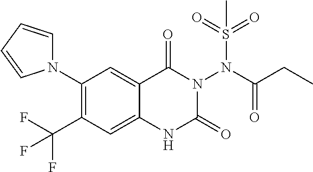 [SMILES: CCC(=O)N(n1c(=O)[nH]c2cc(C(F)(F)F)c(-n3cccc3)cc2c1=O)S(C)(=O)=O]

N-(2,4-dioxo-6-pyrrol-1-yl-7-trifluoromethyl-1,4-dihydro-2H-quinazolin-3-yl)-methanesulfonamide (194.0 mg, 0.5 mmol) is suspended in triethylamine (63.2 mg, 0.625 mmol) and dry dichloro methane (2 mL) at room temperature (22° C.). Sequentially propionyl chloride (50.9 mg, 0.550 mmol) is added and the reaction mixture is stirred at room temperature for 1 hour. Subsequently the crude reaction mixture is poured onto a flash column and subjected to silica gel flash chromatography (ISCO CombiFlash, 24 g silica gel cartridge, heptan/ethyl acetate gradient, ethyl acetate 0% to 50%) to yield N-(2,4-dioxo-6-pyrrol-1-yl-7-trifluoromethyl-1,4-dihydro-2H-quinazolin-3-yl)-N-propionyl-methanesulfonamide. LC-MS at 216 nm; [M+H] 445; Rt 5.923 min; LC-MS Method IV; ¹H-NMR (600 MHz; DMSO-d⁶) δ ppm 0.98 (t, 3H), 2.46 (m, 2H), 3.59 (s, 3H), 6.27 (m, 2H), 6.97 (m, 2H), 7.73 (s, 1H), 7.94 (s, 1H), 12.60 (s, 1H).

### EXAMPLE 8.02

### N-Butyryl-N-(2,4-dioxo-6-pyrrol-1-yl-7-trifluoromethyl-1,4-dihydro-2H-quinazolin-3-yl)-methanesulfonamide

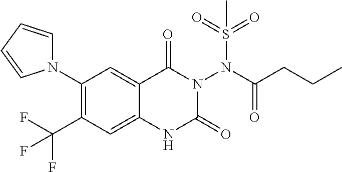 [SMILES: CCCC(=O)N(n1c(=O)[nH]c2cc(C(F)(F)F)c(-n3cccc3)cc2c1=O)S(C)(=O)=O]

Synthesis in analogy to Method A1 starting from N-(2,4-dioxo-6-pyrrol-1-yl-7-triflu oromethyl-1,4-dihydro-2H-quinazolin-3-yl)-methanesulfonamide and butyryl chloride to yield N-butyryl-N-(2,4-dioxo-6-pyrrol-1-yl-7-trifluoromethyl-1,4-dihydro-2H-quinazolin-3-yl)-methanesulfonamide. LC-MS at 216 nm; [M+H] 459; Rt 6.513 min; LC-MS Method IV

### EXAMPLE 8.03

### Acetic acid 2-[(2,4-dioxo-6-pyrrol-1-yl-7-trifluoromethyl-1,4-dihydro-2H-quinazolin-3-yl)-methanesulfonyl-amino]-2-oxo-ethyl ester

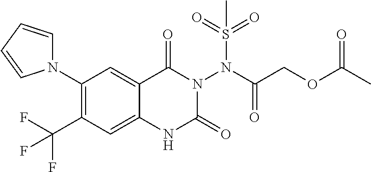 [SMILES: CC(=O)OCC(=O)N(n1c(=O)[nH]c2cc(C(F)(F)F)c(-n3cccc3)cc2c1=O)S(C)(=O)=O]

Synthesis in analogy to Method A1 starting from N-(2,4-dioxo-6-pyrrol-1-yl-7-triflu oromethyl-1,4-dihydro-2H-quinazolin-3-yl)-methanesulfonamide and acetic acid chlorocarbonylmethyl ester to yield acetic acid 2-[(2,4-dioxo-6-pyrrol-1-yl-7-trifluoromethyl-1,4-dihydro-2H-quinazolin-3-yl)-methanesulfonyl-amino]-2-oxo-ethyl ester. LC-MS at 216 nm; [M+H] 489; Rt 6.073 min; LC-MS Method IV

### EXAMPLE 8.04

### (2,4-Dioxo-6-pyrrol-1-yl-7-trifluoromethyl-1,4-dihydro-2H-quinazolin-3-yl)-methanesulfonyl-carbamic acid methyl ester

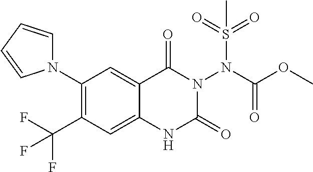 [SMILES: COC(=O)N(n1c(=O)[nH]c2cc(C(F)(F)F)c(-n3cccc3)cc2c1=O)S(C)(=O)=O]

Synthesis in analogy to Method A1 starting from N-(2,4-dioxo-6-pyrrol-1-yl-7-triflu oromethyl-1,4-dihydro-2H-quinazolin-3-yl)-methanesulfonamide and methyl chloroformate to yield (2,4-dioxo-6-pyrrol-1-yl-7-trifluoromethyl-1,4-dihydro-2H-quinazolin-3-yl)-methanesulfonyl-carbamic acid methyl ester. LC-MS at 216 nm; [M+H] 447; Rt 5.128 min; LC-MS Method V

### EXAMPLE 8.05

### (2,4-Dioxo-6-pyrrol-1-yl-7-trifluoromethyl-1,4-dihydro-2H-quinazolin-3-yl)-methanesulfonyl-carbamic acid ethyl ester

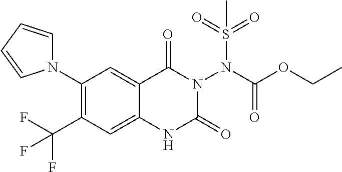 [SMILES: CCOC(=O)N(n1c(=O)[nH]c2cc(C(F)(F)F)c(-n3cccc3)cc2c1=O)S(C)(=O)=O]

Synthesis in analogy to Method A1 starting from N-(2,4-dioxo-6-pyrrol-1-yl-7-triflu oromethyl-1,4-dihydro-2H-quinazolin-3-yl)-methanesulfonamide and ethyl chloroformate to yield (2,4-dioxo-6-pyrrol-1-yl-7-trifluoromethyl-1,4-dihydro-2H-quinazolin-3-yl)-methanesulfonyl-carbamic acid ethyl ester. LC-MS at 216 nm; [M+H] 461; Rt 5.369 min; LC-MS Method V

### EXAMPLE 9.01

### N-(2,4-Dioxo-6-[1,2,4]triazol-4-yl-7-trifluoromethyl-1,4-dihydro-2H-quinazolin-3-yl)-N-(3-methyl-butyryl)-methanesulfonamide

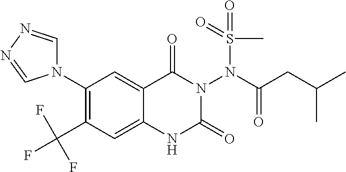 [SMILES: CC(C)CC(=O)N(n1c(=O)[nH]c2cc(C(F)(F)F)c(-n3cnnc3)cc2c1=O)S(C)(=O)=O]

N-(2,4-dioxo-6-[1,2,4]triazol-4-yl-7-trifluoromethyl-1,4-dihydro-2H-quinazolin-3-yl)-methanesulfonamide (195.0 mg, 0.5 mmol) is dissolved in pyridine (2 mL) at room temperature (2° C.). Sequentially 3-methyl-butyryl chloride (60.3 mg, 0.5 mmol) is added and the reaction mixture is stirred at room temperature for 0.25 hour. Subsequently the solvent is evaporated and the crude product is purified by silica gel flash chromatography (ISCO CombiFlash; 12 g silica gel cartridge; cyclohexane/ethyl acetate gradient: ethyl acetate 0% to 100%) to yield N-(2,4-dioxo-6-[1,2,4]triazol-4-yl-7-trifluoromethyl-1,4-dihydro-2H-quinazolin-3-yl)-N-(3-methyl-butyryl)-methanesulfonamide. LC-MS at 254 nm; [M+H] 475; Rt 4.160 min; LC-MS Method III; ¹H-NMR (600 MHz; DMSO-d⁶) δ ppm 0.88 (dd, 6H), 2.04 (m, 1H), 2.33 (m, 2H), 3.60 (s, 3H), 7.77 (s, 1H), 8.33 (s, 1H), 8.79 (s, 2H), 12.70 (s, 1H).

### EXAMPLE 9.02

### N-(2,4-Dioxo-6-[1,2,4]triazol-4-yl-7-trifluoromethyl-1,4-dihydro-2H-quinazolin-3-yl)-N-hexanoyl-methanesulfonamide

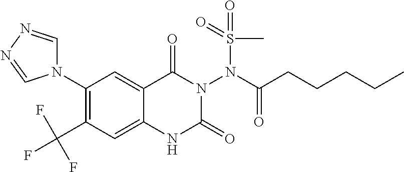 [SMILES: CCCCCC(=O)N(n1c(=O)[nH]c2cc(C(F)(F)F)c(-n3cnnc3)cc2c1=O)S(C)(=O)=O]

Synthesis in analogy to Method C1 starting from N-(2,4-dioxo-6-[1,2,4]triazol-4-yl-7-trifluoromethyl-1,4-dihydro-2H-quinazolin-3-yl)-methanesulfonamide and hexanoyl chloride to yield N-(2,4-dioxo-6-[1,2,4]triazol-4-yl-7-trifluoromethyl-1,4-dihydro-2H-quinazolin-3-yl)-N-hexanoyl-methanesulfonamide. LC-MS at 254 nm; [M+H] 489; Rt 5.050 min; LC-MS Method III

### EXAMPLE 9.03

### N-(2,4-Dioxo-6-[1,2,4]triazol-4-yl-7-trifluoromethyl-1,4-dihydro-2H-quinazolin-3-yl)-N-pentanoyl-methanesulfonamide

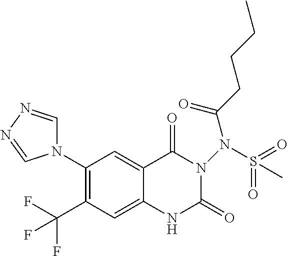 [SMILES: CCCCC(=O)N(n1c(=O)[nH]c2cc(C(F)(F)F)c(-n3cnnc3)cc2c1=O)S(C)(=O)=O]

Synthesis in analogy to Method E1 starting from N-(2,4-dioxo-6-[1,2,4]triazol-4-yl-7-trifluoromethyl-1,4-dihydro-2H-quinazolin-3-yh-methanesulfonamide and pentanoyl chloride to yield N-(2,4-dioxo-6-[1,2,4]triazol-4-yl-7-trifluoromethyl-1,4-dihydro-2H-quinazolin-3-yh-N-pentanoyl-methanesulfonamide. LC-MS at 254 nm; [M+H] 475; Rt 7.224 min; LC-MS Method I

### EXAMPLE 9.04

### N-Butyryl-N-(2,4-dioxo-6-[1,2,4]triazol-4-yl-7-trifluoromethyl-1,4-dihydro-2H-quinazolin-3-yl)-methanesulfonamide

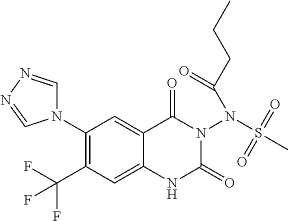 [SMILES: CCCC(=O)N(n1c(=O)[nH]c2cc(C(F)(F)F)c(-n3cnnc3)cc2c1=O)S(C)(=O)=O]

Synthesis in analogy to Method C1 starting from N-(2,4-dioxo-6-[1,2,4]triazol-4-yl-7-trifluoromethyl-1,4-dihydro-2H-quinazolin-3-yh-methanesulfonamide and butyryl chloride to yield N-butyryl-N-(2,4-dioxo-6-[1,2,4]triazol-4-yl-7-trifluoromethyl-1,4-dihydro-2H-quinazolin-3-yl]-methanesulfonamide. LC-MS at 254 nm; [M+H] 461; Rt 6.918 min; LC-MS Method I

### EXAMPLE 10.01

### N-Acetyl-N—((R)-2,4-dioxo-6-tetrahydro-furan-2-yl-7-trifluoromethyl-1,4-dihydro-2H-quinazolin-3-yl)-methanesulfonamide

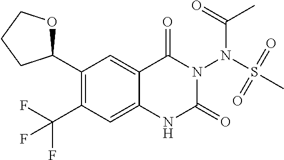 [SMILES: CC(=O)N(n1c(=O)[nH]c2cc(C(F)(F)F)c([C@H]3CCCO3)cc2c1=O)S(C)(=O)=O]

N—((R)-2,4-dioxo-6-tetrahydro-furan-2-yl-7-trifluoromethyl-1,4-dihydro-2H-quinazolin-3-yl)-methanesulfonamide (3.00 g, 7.63 mmol) was dissolved in CH₂Cl₂ (100 ml) at room temperature (22° C.). Pyridine (0.77 ml, 9.53 mmol) was added, followed by acetyl chloride (0.60 ml, 8.39 mmol). The reaction mixture was stirred for 18 h at room temperature (22° C.), and was then concentrated in vacuo. The residue was purified by column chromatography (70 g silica gel; hexane/EtOAc in a ratio of 1/1) to give an oil, which was completely dissolved in tert-butylmethylether. Pentane was then added in order to precipitate N-acetyl-N—((R)-2,4-dioxo-6-tetrahydro-furan-2-yl-7-trifluoromethyl-1,4-dihydro-2H-quinazolin-3-yl)-methanesulfonamide (2.86 g, 6.57 mmol, 86%) as a white solid. LC-MS at 254 nm; [M+NH₄] 453; Rt 0.97 min; (UPLC-MS Method IX). ¹H-NMR (600 MHz; DMSO-d⁶) δ ppm 1.63 (m, 1H), 2.00 (m, 2H), 2.15 (s, 3H), 2.34 (m, 1H), 3.59 (s, 3H), 3.86 (m, 1H), 4.15 (m, 1H), 5.05 (m, 1H), 7.58 (s, 1H), 8.26 (s, 1H), 12.38 (s, 1H).

### EXAMPLE 10.02

### N—((R)-2,4-Dioxo-6-tetrahydro-furan-2-yl-7-trifluoromethyl-1,4-dihydro-2H-quinazolin-3-yl)-N-propionyl-methanesulfonamide

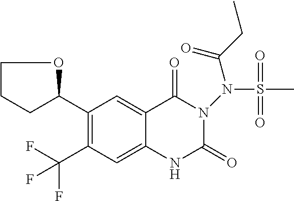 [SMILES: CCC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and propionyl chloride to yield N—((R)-2,4-dioxo-6-tetrahydro-furan-2-yl-7-trifluoromethyl-1,4-dihydro-2H-quinazolin-3-yl)-N-propionyl-methanesulfonamide. LC-MS at 254 nm; [M+NH₄] 467; Rt 1.11 min; (LC-MS Method VI)

### EXAMPLE 10.03

### N-Butyryl-N—((R)-2,4-dioxo-6-tetrahydro-furan-2-yl-7-trifluoromethyl-1,4-dihydro-2H-quinazolin-3-yp-methanesulfonamide

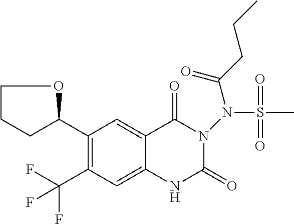 [SMILES: CCCC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and butyryl chloride to yield N-butyryl-N—((R)-2,4-dioxo-6-tetrahydro-furan-2-yl-7-trifluoromethyl-1,4-dihydro-2H-quinazolin-3-yl)-methanesulfonamide. LC-MS at 254 nm; [M+NH₄] 481; Rt 1.15 min; (LC-MS Method VI)

### EXAMPLE 10.04

### Methanesulfonyl-N—((R)-2,4-dioxo-6-tetrahydro-furan-2-yl-7-trifluoromethyl-1,4-dihydro-2H-quinazolin-3-yl)-carbamic acid methyl ester

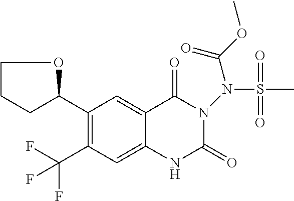 [SMILES: COC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and methyl chloroformate to yield methanesulfonyl-N—((R)-2,4-dioxo-6-tetrahydro-furan-2-yl-7-trifluoromethyl-1,4-dihydro-2H-quinazolin-3-yl)-carbamic acid methyl ester. LC-MS at 254 nm; [M+NH₄] 469; Rt 1.07 min; (LC-MS Method VI)

### EXAMPLE 10.05

### Methanesulfonyl-N—((R)-2,4-dioxo-6-tetrahydro-furan-2-yl-7-trifluoromethyl-1,4-dihydro-2H-quinazolin-3-yl)-carbamic acid ethyl ester

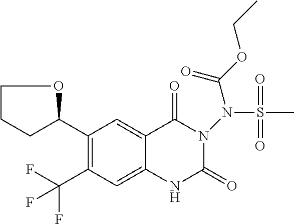 [SMILES: CCOC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and ethyl chloroformate to yield methanesulfonyl-N—((R)-2,4-dioxo-6-tetrahydro-furan-2-yl-7-trifluoromethyl-1,4-dihydro-2H-quinazolin-3-yl)-carbamic acid ethyl ester. LC-MS at 254 nm; [M+NH₄] 483; Rt 1.10 min; (LC-MS Method VI)

### EXAMPLE 10.06

### Methanesulfonyl-N—((R)-2,4-dioxo-6-tetrahydro-furan-2-yl-7-trifluoromethyl-1,4-dihydro-2H-quinazolin-3-yl)-carbamic acid butyl ester

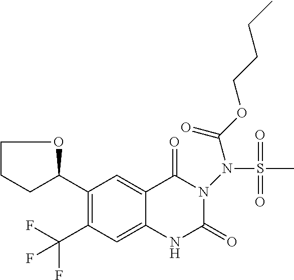 [SMILES: CCCCOC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and butyl chloroformate to yield methanesulfonyl-N—((R)-2,4-dioxo-6-tetrahydro-furan-2-yl-7-trifluoromethyl-1,4-dihydro-2H-quinazolin-3-yl)-carbamic acid butyl ester. LC-MS at 254 nm; [M+NH₄] 511; Rt 1.20 min; (LC-MS Method VI)

### EXAMPLE 10.07

### Methanesulfonyl-N—((R)-2,4-dioxo-6-tetrahydro-furan-2-yl-7-trifluoromethyl-1,4-dihydro-2H-quinazolin-3-yl)-carbamic acid 2-methoxy-ethyl ester

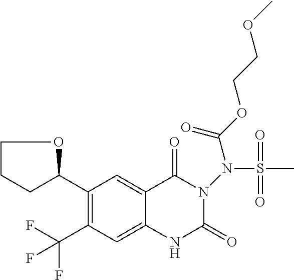 [SMILES: COCCOC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and 2-methoxy-ethyl chloroformate to yield methanesulfonyl-N—((R)-2,4-dioxo-6-tetrahydro-furan-2-yl-7-trifluoromethyl-1,4-dihydro-2H-quinazolin-3-yl)-carbamic acid 2-methoxy-ethyl ester. LC-MS at 254 nm; [M+NH₄] 513; Rt 1.10 min; (LC-MS Method VI)

### EXAMPLE 10.08

### Methanesulfonyl-N—((R)-2,4-dioxo-6-tetrahydro-furan-2-yl-7-trifluoromethyl-1,4-dihydro-2H-quinazolin-3-yl)-carbamic acid isobutyl ester

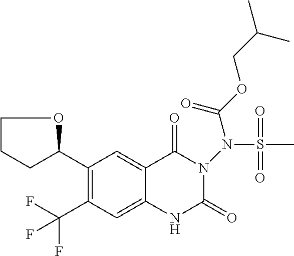 [SMILES: CC(C)COC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and isobutyl chloroformate to yield methanesulfonyl-N—((R)-2,4-dioxo-6-tetrahydro-furan-2-yl-7-trifluoromethyl-1,4-dihydro-2H-quinazolin-3-yl)-carbamic acid isobutyl ester. LC-MS at 254 nm; [M+NH₄] 511; Rt 1.20 min; (LC-MS Method VI)

### EXAMPLE 10.09

### N—((R)-2,4-Dioxo-6-tetrahydro-furan-2-yl-7-trifluoromethyl-1,4-dihydro-2H-quinazolin-3-yl)-N-hexanoyl-methanesulfonamide

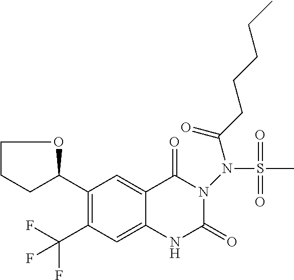 [SMILES: CCCCCC(=O)N(n1c(=O)[nH]c2cc(C(F)(F)F)c([C@H]3CCCO3)cc2c1=O)S(C)(=O)=O]

Synthesis in analogy to Method B1 starting from N—((R)-2,4-dioxo-6-tetrahydro-furan-2-yl-7-trifluoromethyl-1,4-dihydro-2H-quinazolin-3-yl)-methanesulfonamide and hexanoyl chloride to yield N—((R)-2,4-dioxo-6-tetrahydro-furan-2-yl-7-trifluoromethyl-1,4-dihydro-2H-quinazolin-3-yl)-N-hexanoyl-methanesulfonamide. LC-MS at 254 nm; [M+NH₄] 509; Rt 1.24 min; (LC-MS Method VI)

### EXAMPLE 10.10

### 4-R(R)-2,4-Dioxo-6-tetrahydro-furan-2-yl-7-trifluoromethyl-1,4-dihydro-2H-quinazolin-3-yl)-methanesulfonyl-amino]-4-oxo-butyric acid ethyl ester

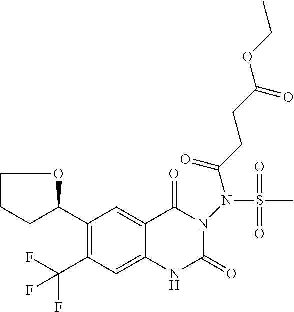 [SMILES: CCOC(=O)CCC(=O)N(n1c(=O)[nH]c2cc(C(F)(F)F)c([C@H]3CCCO3)cc2c1=O)S(C)(=O)=O]

Synthesis in analogy to Method E2 starting from N—((R)-2,4-dioxo-6-tetrahydro-furan-2-yl-7-trifluoromethyl-1,4-dihydro-2H-quinazolin-3-yl)-methanesulfonamide and 3-chlorocarbonyl-propionic acid ethyl ester to yield 4-R(R)-2,4-dioxo-6-tetrahydro-furan-2-yl-7-trifluoromethyl-1,4-dihydro-2H-quinazolin-3-yl)-methanesulfonyl-amino]-4-oxo-butyric acid ethyl ester. LC-MS at 254 nm; [M+NH₄] 522; Rt 8.486 min; (LC-MS Method I)

### EXAMPLE 10.11

### N—((R)-2,4-Dioxo-6-tetrahydro-furan-2-yl-7-trifluoromethyl-1,4-dihydro-2H-quinazolin-3-yl)-N-pentanoyl-methanesulfonamide

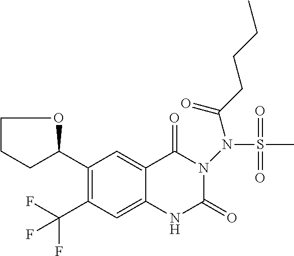 [SMILES: CCCCC(=O)N(n1c(=O)[nH]c2cc(C(F)(F)F)c([C@H]3CCCO3)cc2c1=O)S(C)(=O)=O]

Synthesis in analogy to Method E2 starting from N—((R)-2,4-dioxo-6-tetrahydro-furan-2-yl-7-trifluoromethyl-1,4-dihydro-2H-quinazolin-3-yl)-methanesulfonamide and pentanoyl chloride to yield N—((R)-2,4-dioxo-6-tetrahydro-furan-2-yl-7-trifluoromethyl-1,4-dihydro-2H-quinazolin-3-yl)-N-pentanoyl-methanesulfonamide. LC-MS at 254 nm; [M+NH₄] 478; Rt 9.571 min; (LC-MS Method I)

### EXAMPLE 11.01

### Methanesulfonyl-[6-(1-methoxy-propyl)-2,4-dioxo-7-trifluoromethyl-1,4-dihydro-2H-quinazolin-3-yl]-carbamic acid 2-methoxy-ethyl ester

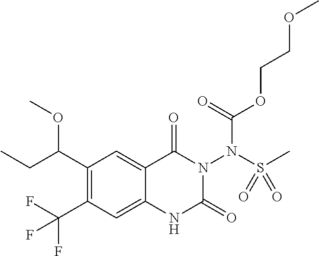 [SMILES: CCC(OC)c1cc2c(=O)n(N(C(=O)OCCOC)S(C)(=O)=O)c(=O)[nH]c2cc1C(F)(F)F]

N-[6-(1-methoxy-propyl)-2,4-dioxo-7-trifluoromethyl-1,4-dihydro-2H-quinazolin-3-yl]-methanesulfonamide (140 mg, 333 μmol) was dissolved in DMF (3 ml) at room temperature (22° C.). NaH (17 mg, 399 μmol) was added, and stirring was continued for 30 min. 2-Methoxyethylcarbonochloridate (58 μl, 399 μmol) was added, and the reaction mixture was stirred for 18 h at room temperature (22° C.). The reaction was quenched by pouring the mixture on ice-cooled water. After dilution with EtOAc, the organic solvents were separated and washed with water and brine. The organic solvents were combined, dried over MgSO4, filtered, and concentrated in vacuo. The residue was purified by column chromatography (4 g silica gel; hexane/EtOAc in a ratio of 9/1) to give methanesulfonyl-[6-(1-methoxy-propyl)-2,4-dioxo-7-trifluoromethyl-1,4-dihydro-2H-quinazolin-3-yl]-carbamic acid 2-methoxy-ethyl ester (60 mg, 119 μmol, 36% yield) as a slightly yellow oil. LC-MS at 254 nm; [M+NH₄] 515; Rt 1.13 min; (LC-MS Method VI). ¹H-NMR (600 MHz; DMSO-d⁶) δ ppm 0.91 (t, J=7.3 Hz, 3H), 1.65 (m, 2H), 3.12 (s, 3H), 3.27 (m, 2H), 3.63 (m, 3H), 3.66 (s, 3H), 4.37 (m, 1H), 4.43 (m, 2H), 7.61 (s, 1H), 8.17 (s, 1H), 12.43 (s, 1H).

### EXAMPLE 11.02

### N-[6-(1-Methoxy-propyl)-2,4-dioxo-7-trifluoromethyl-1,4-dihydro-2H-quinazolin-3-yl]-N-propionyl-methanesulfonamide

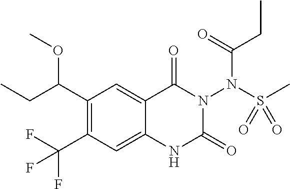 [SMILES: CCC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and propionyl chloride to yield N-[6-(1-methoxy-propyl)-2,4-dioxo-7-trifluoromethyl-1,4-dihydro-2H-quinazolin-3-yl]-N-propionyl-methanesulfonamide. LC-MS at 254 nm; [M+NH₄] 469; Rt 1.15 min; (LC-MS Method VI)

### EXAMPLE 11.03

### Methanesulfonyl-[6-(1-methoxy-propyl)-2,4-dioxo-7-trifluoromethyl-1,4-dihydro-2H-quinazolin-3-yl]-carbamic acid ethyl ester

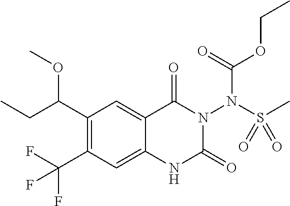 [SMILES: CCOC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and ethyl chloroformate to yield methanesulfonyl-[6-(1-methoxy-propyl)-2,4-dioxo-7-trifluoromethyl-1,4-dihydro-2H-quinazolin-3-yl]-carbamic acid ethyl ester. LC-MS at 254 nm; [M+NH₄] 485; Rt 1.15 min; (LC-MS Method VI)

### EXAMPLE 11.04

### Methanesulfonyl-[6-(1-methoxy-propyl)-2,4-dioxo-7-trifluoromethyl-1,4-dihydro-2H-quinazolin-3-yl]-carbamic acid propyl ester

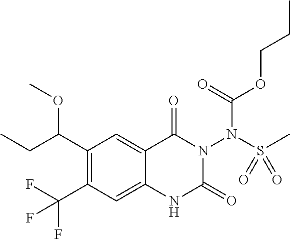 [SMILES: CCCOC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and propyl chloroformate to yield methanesulfonyl-[6-(1-methoxy-propyl)-2,4-dioxo-7-trifluoromethyl-1,4-dihydro-2H-quinazolin-3-yl]-carbamic acid propyl ester. LC-MS at 254 nm; [M+NH₄] 499; Rt 1.20 min; (LC-MS Method VI)

### EXAMPLE 11.05

### N-Hexanoyl-N-[6-(1-Methoxy-propyl)-2,4-dioxo-7-trifluoromethyl-1,4-dihydro-2H-quinazolin-3-yl]-methanesulfonamide

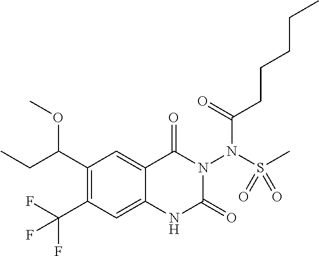 [SMILES: CCCCCC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and hexanoyl chloride to yield N-hexanoyl-N-[6-(1-methoxy-propyl)-2,4-dioxo-7-trifluoromethyl-1,4-dihydro-2H-quinazolin-3-yl]-methanesulfonamide. LC-MS at 254 nm; [M+NH₄] 511; Rt 1.27 min; (LC-MS Method VI)

### EXAMPLE 11.06

### N-Isobutyryl-N-[6-(1-methoxy-propyl)-2,4-dioxo-7-trifluoromethyl-1,4-dihydro-2H-quinazolin-3-yl]-methanesulfonamide

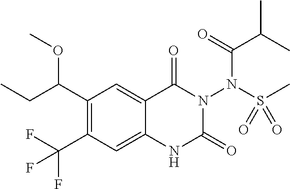 [SMILES: CCC(OC)c1cc2c(=O)n(N(C(=O)C(C)C)S(C)(=O)=O)c(=O)[nH]c2cc1C(F)(F)F]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and isobutyryl chloride to yield N-isobutyryl-N-[6-(1-methoxy-propyl)-2,4-dioxo-7-trifluoromethyl-1,4-dihydro-2H-quinazolin-3-yl]-methanesulfonamide. LC-MS at 254 nm; [M+NH₄] 483; Rt 1.18 min; (LC-MS Method VI)

### EXAMPLE 11.07

### N-Butyryl-N-[6-(1-methoxy-propyl)-2,4-dioxo-7-trifluoromethyl-1,4-dihydro-2H-quinazolin-3-yl]-methanesulfonamide

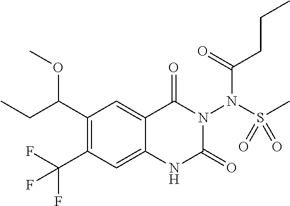 [SMILES: CCCC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and butyryl chloride to yield N-butyryl-N-[6-(1-methoxy-propyl)-2,4-dioxo-7-trifluoromethyl-1,4-dihydro-2H-quinazolin-3-yl]-methanesulfonamide. LC-MS at 254 nm; [M+NH₄] 483; Rt 1.18 min; (LC-MS Method VI)

### EXAMPLE 11.08

### N-Acetyl-N-[6-(1-methoxy-propyl)-2,4-dioxo-7-trifluoromethyl-1,4-dihydro-2H-quinazolin-3-yl]-methanesulfonamide

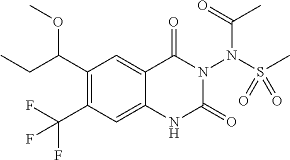 [SMILES: CCC(OC)c1cc2c(=O)n(N(C(C)=O)S(C)(=O)=O)c(=O)[nH]c2cc1C(F)(F)F]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and acetyl chloride to yield N-acetyl-N-[6-(1-methoxy-propyl)-2,4-dioxo-7-trifluoromethyl-1,4-dihydro-2H-quinazolin-3-yl]-methanesulfonamide. LC-MS at 254 nm; [M+NH₄] 455; Rt 1.10 min; (LC-MS Method VI)

### EXAMPLE 11.09

### Methanesulfonyl-[6-(1-methoxy-propyl)-2,4-dioxo-7-trifluoromethyl-1,4-dihydro-2H-quinazolin-3-yl]-carbamic acid methyl ester

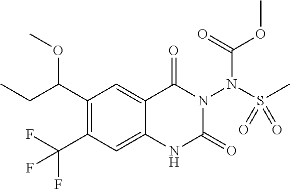 [SMILES: CCC(OC)c1cc2c(=O)n(N(C(=O)OC)S(C)(=O)=O)c(=O)[nH]c2cc1C(F)(F)F]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and methyl chloroformate to yield methanesulfonyl-[6-(1-methoxy-propyl)-2,4-dioxo-7-trifluoromethyl-1,4-dihydro-2H-quinazolin-3-yl]-carbamic acid methyl ester. LC-MS at 254 nm; [M+NH₄] 471; Rt 1.12 min; (LC-MS Method VI)

### EXAMPLE 11.10

### Methanesulfonyl-[6-(1-methoxy-propyl)-2,4-dioxo-7-trifluoromethyl-1,4-dihydro-2H-quinazolin-3-yl]-carbamic acid isobutyl ester

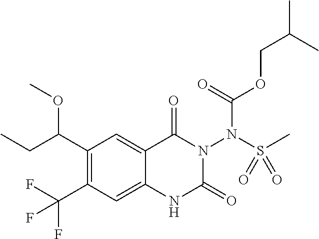 [SMILES: CCC(OC)c1cc2c(=O)n(N(C(=O)OCC(C)C)S(C)(=O)=O)c(=O)[nH]c2cc1C(F)(F)F]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and isobutyl chloroformate to yield methanesulfonyl-[6-(1-methoxy-propyl)-2,4-dioxo-7-trifluoromethyl-1,4-dihydro-2H-quinazolin-3-yl]-carbamic acid isobutyl ester. LC-MS at 254 nm; [M+NH₄] 513; Rt 1.24 min; (LC-MS Method VI)

### EXAMPLE 11.11

### Methanesulfonyl-[6-(1-methoxy-propyl)-2,4-dioxo-7-trifluoromethyl-1,4-dihydro-2H-quinazolin-3-yl]-carbamic acid butyl ester

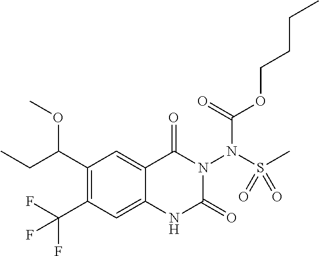 [SMILES: CCCCOC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and butyl chloroformate to yield methanesulfonyl-[6-(1-methoxy-propyl)-2,4-dioxo-7-trifluoromethyl-1,4-dihydro-2H-quinazolin-3-yl]-carbamic acid butyl ester. LC-MS at 254 nm; [M+H] 496; Rt 3.481 min; (LC-MS Method VIII)

### EXAMPLE 11.12

### Methanesulfonyl-[6-(1-methoxy-propyl)-2,4-dioxo-7-trifluoromethyl-1,4-dihydro-2H-quinazolin-3-yl]-carbamic acid pentyl ester

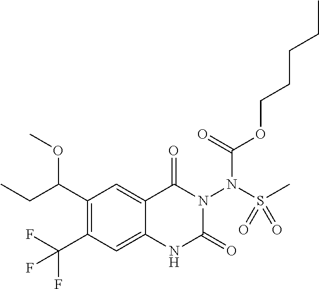 [SMILES: CCCCCOC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and pentyl chloroformate to yield methanesulfonyl-[6-(1-methoxy-propyl)-2,4-dioxo-7-trifluoromethyl-1,4-dihydro-2H-quinazolin-3-yl]-carbamic acid pentyl ester. LC-MS at 254 nm; [M+H] 510; Rt 3.653 min; (LC-MS Method VIII)

### EXAMPLE 11.13

### Methanesulfonyl-[6-(1-methoxy-propyl)-2,4-dioxo-7-trifluoromethyl-1,4-dihydro-2H-quinazolin-3-yl]-carbamic acid hexyl ester

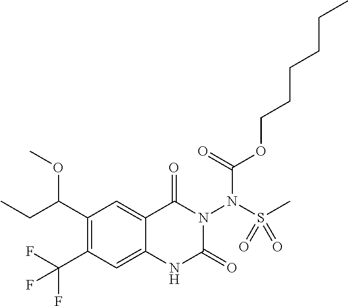 [SMILES: CCCCCCOC(=O)N(n1c(=O)[nH]c2cc(C(F)(F)F)c(C(CC)OC)cc2c1=O)S(C)(=O)=O]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and hexyl chloroformate to yield methanesulfonyl-[6-(1-methoxy-propyl)-2,4-dioxo-7-trifluoromethyl-1,4-dihydro-2H-quinazolin-3-yl]-carbamic acid hexyl ester. LC-MS at 254 nm; [M+H] 524; Rt 3.832 min; (LC-MS Method VIII)

### EXAMPLE 11.14

### Methanesulfonyl-[6-(1-methoxy-propyl)-2,4-dioxo-7-trifluoromethyl-1,4-dihydro-2H-quinazolin-3-yl]-carbamic acid isopropyl ester

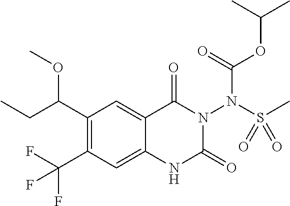 [SMILES: CCC(OC)c1cc2c(=O)n(N(C(=O)OC(C)C)S(C)(=O)=O)c(=O)[nH]c2cc1C(F)(F)F]

Synthesis in analogy to Method B1 starting from N-[6-(1-methoxy-propyl)-2,4-dioxo-7-trifluoromethyl-1,4-dihydro-2H-quinazolin-3-yl]-methanesulfonamide and isopropyl chloroformate to yield methanesulfonyl-[6-(1-methoxy-propyl)-2,4-dioxo-7-trifluoromethyl-1,4-dihydro-2H-quinazolin-3-yl]-carbamic acid isopropyl ester. LC-MS at 254 nm; [M+NH₄] 499; Rt 3.280 min; (LC-MS Method VIII)

### EXAMPLE 11.15

### N-[6-(1-methoxy-propyl)-2,4-dioxo-7-trifluoromethyl-1,4-dihydro-2H-quinazolin-3-yl]-N-pentanoyl-methanesulfonamide

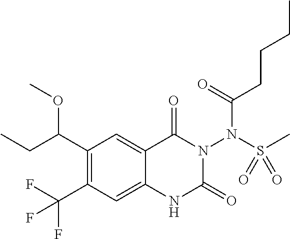 [SMILES: CCCCC(=O)N(n1c(=O)[nH]c2cc(C(F)(F)F)c(C(CC)OC)cc2c1=O)S(C)(=O)=O]

Synthesis in analogy to Method B1 starting from N-[6-(1-Methoxy-propyl)-2,4-dioxo-7-trifluoromethyl-1,4-dihydro-2H-quinazolin-3-yl]-methanesulfonamide and pentanoyl chloride to yield N-[6-(1-methoxy-propyl)-2,4-dioxo-7-trifluoromethyl-1,4-dihydro-2H-quinazolin-3-yl]-N-pentanoyl-methanesulfonamide. LC-MS at 254 nm; [M+NH₄] 497; Rt 3.440 min; (LC-MS Method VIII)

### EXAMPLE 12.01

### N-Hexanoyl-N-[6-(1-hydroxy-propyl)-2,4-dioxo-7-trifluoromethyl-1,4-dihydro-2H-quinazolin-3-yl]-methanesulfonamide

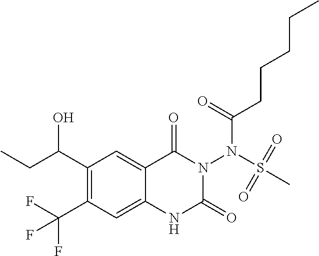 [SMILES: CCCCCC(=O)N(n1c(=O)[nH]c2cc(C(F)(F)F)c(C(O)CC)cc2c1=O)S(C)(=O)=O]

N-[6-(1-hydroxy-propyl)-2,4-dioxo-7-trifluoromethyl-1,4-dihydro-2H-quinazolin-3-yl]-methanesulfonamide (100 mg, 262 mmol) was dissolved in DMF (2 ml) at room temperature (22° C.). NaH (17 mg, 393 mmol) was added, and stirring was continued for 30 min. Hexanoyl chloride (53 mg, 393 mmol) was added, and the reaction mixture was stirred for 5 h at room temperature (22° C.). The reaction was quenched by pouring the mixture on ice-cooled water. After dilution with EtOAc, the organic solvents were separated and washed with water and brine. The organic solvents were combined, dried over MgSO4, filtered, and concentrated in vacuo. The residue was purified by preparative HPLC (Gilson prep. HPLC; Waters Sunfire C18, 5 μm, 30×100 mm) using as eluent 0.1% TFA/(acetonitril+0.1% TFA) gradient from 70:30 to 30:70 in 17 min, the relevant fractions were concentrated and the residue diluted with 2 ml of diethyl ether to precipitate N-hexanoyl-N-[6-(1-hydroxy-propyl)-2,4-dioxo-7-trifluoromethyl-1,4-dihydro-2H-quinazolin-3-yl]-methanesulfonamide (59 mg, 122 μmol, 46% yield) as a slightly yellow solid. LC-MS at 254 nm; [M+NH₄] 497; Rt 1.40 min; (LC-MS Method VI). ¹H-NMR (600 MHz; DMSO-d⁶) δ ppm 0.83 (t, J=6.0 Hz, 3H), 0.94 (t, J=7.2 Hz, 3H), 1.22 (m, 2H), 1.23 (m, 2H), 1.51 (m, 2H), 1.59 (m, 2H), 2.42 (m, 2H), 3.57 (s, 3H), 4.73 (m, 1H), 5.65 (s, 1H), 7.54 (s, 1H), 8.35 (s, 1H), 12.31 (s, 1H).

### EXAMPLE 12.02

### Methanesulfonyl-[6-(1-hydroxy-propyl)-2,4-dioxo-7-trifluoromethyl-1,4-dihydro-2H-quinazolin-3-yl]-carbamic acid 2-methoxy-ethyl ester

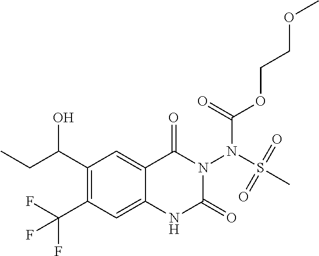 [SMILES: CCC(O)c1cc2c(=O)n(N(C(=O)OCCOC)S(C)(=O)=O)c(=O)[nH]c2cc1C(F)(F)F]

Synthesis in analogy to Method B1 starting from N-[6-(1-hydroxy-propyl)-2,4-dioxo-7-trifluoromethyl-1,4-dihydro-2H-quinazolin-3-yl]-methanesulfonamide and 2-methoxy-ethyl chloroformate to yield methanesulfonyl-[6-(1-hydroxy-propyl)-2,4-dioxo-7-trifluoromethyl-1,4-dihydro-2H-quinazolin-3-yl]-carbamic acid 2-methoxy-ethyl ester. LC-MS at 254 nm; [M+NH₄] 501; Rt 1.21 min; (LC-MS Method VI)

### EXAMPLE 12.03

### N-Butyryl-N-[6-(1-hydroxy-propyl)-2,4-dioxo-7-trifluoromethyl-1,4-dihydro-2H-quinazolin-3-yl]-methanesulfonamide

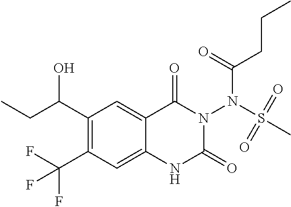 [SMILES: CCCC(=O)N(n1c(=O)[nH]c2cc(C(F)(F)F)c(C(O)CC)cc2c1=O)S(C)(=O)=O]

Synthesis in analogy to Method B1 starting from N-[6-(1-hydroxy-propyl)-2,4-dioxo-7-trifluoromethyl-1,4-dihydro-2H-quinazolin-3-yl]-methanesulfonamide and butyryl chloride to yield N-butyryl-N-[6-(1-hydroxy-propyl)-2,4-dioxo-7-trifluoromethyl-1,4-dihydro-2H-quinazolin-3-yl]-methanesulfonamide. LC-MS at 254 nm; [M+NH₄] 469; Rt 1.16 min; (LC-MS Method VI)

### EXAMPLE 12.04

### N-[6-(1-Hydroxy-propyl)-2,4-dioxo-7-trifluoromethyl-1,4-dihydro-2H-quinazolin-3-yl]-N-isobutyryl-methanesulfonamide

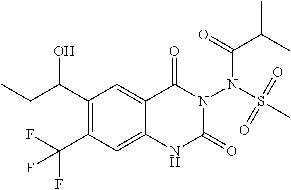 [SMILES: CCC(O)c1cc2c(=O)n(N(C(=O)C(C)C)S(C)(=O)=O)c(=O)[nH]c2cc1C(F)(F)F]

Synthesis in analogy to Method B1 starting from N-[6-(1-hydroxy-propyl)-2,4-dioxo-7-trifluoromethyl-1,4-dihydro-2H-quinazolin-3-yl]-methanesulfonamide and isobutyryl chloride to yield N-[6-(1-hydroxy-propyl)-2,4-dioxo-7-trifluoromethyl-1,4-dihydro-2H-quinazolin-3-yl]-N-isobutyryl-methanesulfonamide. LC-MS at 254 nm; [M+NH₄] 469; Rt 1.15 min; (LC-MS Method VI)

### EXAMPLE 12.05

### N-[6-(1-Hydroxy-propyl)-2,4-dioxo-7-trifluoromethyl-1,4-dihydro-2H-quinazolin-3-yl]-N-pentanoyl-methanesulfonamide

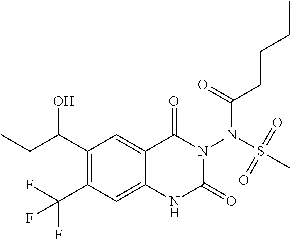 [SMILES: CCCCC(=O)N(n1c(=O)[nH]c2cc(C(F)(F)F)c(C(O)CC)cc2c1=O)S(C)(=O)=O]

Synthesis in analogy to Method B1 starting from N-[6-(1-hydroxy-propyl)-2,4-dioxo-7-trifluoromethyl-1,4-dihydro-2H-quinazolin-3-yl]-methanesulfonamide and pentanoyl chloride to yield N-[6-(1-hydroxy-propyl)-2,4-dioxo-7-trifluoromethyl-1,4-dihydro-2H-quinazolin-3-yl]-N-pentanoyl-methanesulfonamide. LC-MS at 254 nm; [M+NH₄] 483; Rt 1.22 min; (LC-MS Method VI)

### EXAMPLE 13.01

### Methanesulfonyl-[7-ethyl-6-(2-ethyl-2H-pyrazol-3-yl)-2,4-dioxo-1,4-dihydro-2H-quinazolin-3-yl]carbamic acid isobutyl ester

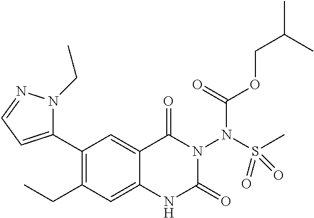 [SMILES: CCc1cc2[nH]c(=O)n(N(C(=O)OCC(C)C)S(C)(=O)=O)c(=O)c2cc1-c1ccnn1CC]

N-[7-ethyl-6-(2-ethyl-2H-pyrazol-3-yl)-2,4-dioxo-1,4-dihydro-2H-quinazolin-3-yl]-methanesulfonamide (150 mg, 397 μmol) was dissolved in DMF (3 ml) at room temperature (22° C.). NaH (21 mg, 477 μmol) was added, and stirring was continued for 30 min. Isobutyl carbonochloridate (65 mg, 477 μmol) was added, and the reaction mixture was stirred for 18 h at room temperature (22° C.). The reaction was quenched by pouring the mixture on ice-cooled water. After dilution with EtOAc, the organic solvents were separated and washed with water and brine. The organic solvents were combined, dried over MgSO4, filtered, and concentrated in vacuo. The residue was purified by preparative HPLC (Gilson prep. HPLC; Waters Sunfire C18, 5 pm, 30×100 mm) using as eluent 0.1% TFA/(acetonitril+0.1% TFA) gradient from 60:40 to 40:60 in 17 min, the relevant fractions were concentrated, and the residue was diluted with 2 ml of diethyl ether to precipitate methanesulfonyl-[7-ethyl-6-(2-ethyl-2H-pyrazol-3-yl)-2,4-dioxo-1,4-dihydro-2H-quinazolin-3-yl]carbamic acid isobutyl ester (60 mg, 124 μmol, 31%) as a white solid. LC-MS at 254 nm; [M+H] 478; Rt 1.07 min; (LC-MS method VI). ¹H-NMR (600 MHz; DMSO-d⁶) δ ppm 0.87 (m, 6H), 1.05 (t, J=7.7 Hz, 3H), 1.20 (t, J=7.4 Hz, 3H), 1.94 (m, 1H), 2.50 (m, 2H), 3.63 (s, 3H), 3.84 (q, J=7.4 Hz, 2H), 4.09 (m, 2H), 6.32 (s, 1H), 7.28 (s, 1H), 7.56 (s, 1H), 7.76 (s, 1H), 12.28 (s, 1H).

### EXAMPLE 13.02

### N-[7-Ethyl-6-(2-ethyl-2H-pyrazol-3-yl)-2,4-dioxo-1,4-dihydro-2H-quinazolin-3-yl]-N-hexanoyl-methanesulfonamide

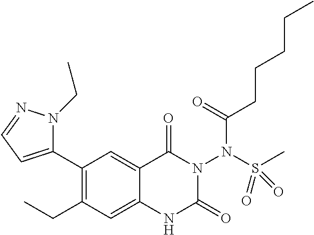 [SMILES: CCCCCC(=O)N(n1c(=O)[nH]c2cc(CC)c(-c3ccnn3CC)cc2c1=O)S(C)(=O)=O]

Synthesis in analogy to Method B1 starting from N-[7-ethyl-6-(2-ethyl-2H-pyrazol-3-yl)-2,4-dioxo-1,4-dihydro-2H-quinazolin-3-yl]-methanesulfonamide and hexanoyl chloride to yield N—[7-ethyl-6-(2-ethyl-2H-pyrazol-3-yl)-2,4-dioxo-1,4-dihydro-2H-quinazolin-3-yl]-N-hexanoyl-methanesulfonamide. LC-MS at 254 nm; [M+H] 476; Rt 1.11 min; (LC-MS method VI)

### EXAMPLE 13.03

### N-[7-Ethyl-6-(2-ethyl-2H-pyrazol-3-yl)-2,4-dioxo-1,4-dihydro-2H-quinazolin-3-yl]-N-propionyl-methanesulfonamide

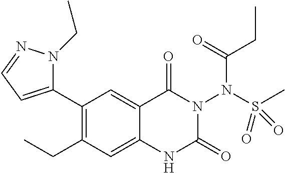 [SMILES: CCC(=O)N(n1c(=O)[nH]c2cc(CC)c(-c3ccnn3CC)cc2c1=O)S(C)(=O)=O]

Synthesis in analogy to Method B1 starting from N-[7-ethyl-6-(2-ethyl-2H-pyrazol-3-yl)-2,4-dioxo-1,4-dihydro-2H-quinazolin-3-yl]-methanesulfonamide and propionyl chloride to yield N-[7-ethyl-6-(2-ethyl-2H-pyrazol-3-yl)-2,4-dioxo-1,4-dihydro-2H-quinazolin-3-yl]-N-propionyl-methanesulfonamide. LC-MS at 254 nm; [M+H] 434; Rt 0.97 min; (LC-MS method VI)

### EXAMPLE 14.01

### Acetic acid 2-{[7-difluoromethyl-6-(2-ethyl-2H-pyrazol-3-yl)-2,4-dioxo-1,4-dihydro-2H-quinazolin-3-yl]-methanesulfonyl-amino}-2-oxo-ethyl ester

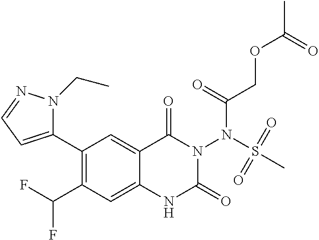 [SMILES: CCn1nccc1-c1cc2c(=O)n(N(C(=O)COC(C)=O)S(C)(=O)=O)c(=O)[nH]c2cc1C(F)F]

N-[7-difluoromethyl-6-(2-ethyl-2H-pyrazol-3-yl)-2,4-dioxo-1,4-dihydro-2H-quinazolin-3-yl]-methanesulfonamide (81.0 mg, 0.203 mmol) was dissolved in dry methylene chloride (1.5 mL) and triethyl amine (25.7 mg, 0.254 mmol) at room temperature. Sequentially acetic acid chlorocarbonylmethyl ester (29.1 mg, 0.213 mmol) was added and the reaction mixture was stirred at room temperature for 10 minutes. Subsequently the reaction mixture was subjected to purification by silica gel flash chromatography (ISCO CombiFlash; 40 g gold standard silica gel cartridge; methylene chloride/methanol gradient: methanol 0% to 5%) to yield acetic acid 2-{[7-difluoromethyl-6-(2-ethyl-2H-pyrazol-3-yl)-2,4-dioxo-1,4-dihydro-2H-quinazolin-3-yl]-methanesulfonyl-amino}-2-oxo-ethyl ester (98.0 mg, 0.192 mmol, 95%). LC-MS at 254 nm; [M+H] 500; Rt 4.554 min; (LC-MS method V); ¹H-NMR (600 MHz; DMSO-d⁶) δ ppm 1.22 (t, 3H), 2.11 (s, 3H), 3.63 (s, 3H), 3.95 (q, 2H), 4.88 (broad s, 2H), 6.37 (s, 1H), 6.88 (t, 1H), 7.59 (s, 1H), 7.64 (s, 1H), 7.99 (s, 1H), 12.60 (s, 1H).

### EXAMPLE 15.01

### [6-(2-Ethyl-2H-pyrazol-3-yl)-2,4-dioxo-7-trifluoromethyl-1,4-dihydro-2H-quinazolin-3-yl]-methanesulfonyl-carbamic acid methyl ester

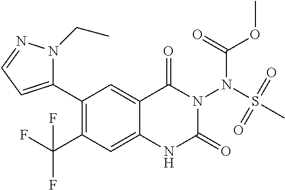 [SMILES: CCn1nccc1-c1cc2c(=O)n(N(C(=O)OC)S(C)(=O)=O)c(=O)[nH]c2cc1C(F)(F)F]

N-[6-(2-ethyl-2H-pyrazol-3-yl)-2,4-dioxo-7-trifluoromethyl-1,4-dihydro-2H-quinazolin-3-yl]-methanesulfonamide (90.0 mg, 0.216 mmol) was dissolved in dry methylene chloride (1.5 mL) and triethyl amine (27.3 mg, 0.270 mmol). Sequentially methyl chloroformate (21.4 mg, 0.226 mmol) was added and the reaction mixture was stirred at room temperature for 15 minutes. Subsequently the reaction mixture was subjected to purification by silica gel flash chromatography (ISCO CombiFlash; 24 g silica gel cartridge; methylene chloride/methanol gradient: methanol 0% to 5%) to yield [6-(2-ethyl-2H-pyrazol-3-yl)-2,4-dioxo-7-trifluoromethyl-1,4-dihydro-2H-quinazolin-3-yl]-methanesulfonyl-carbamic acid methyl ester (69.0 mg, 0.144 mmol, 66.6%). LC-MS at 254 nm; [M+H] 476; Rt 4.737 min; (LC-MS method V); ¹H-NMR (600 MHz; DMSO-d⁶) δ ppm 1.22 (t, 3H), 3.66 (s, 3H), 3.86 (s, 3H), 3.90 (q, 2H), 6.32 (d, 1H), 7.56 (d, 1H), 7.77 (s, 1H), 8.01 (s, 1H), 12.70 (s, 1H).

### EXAMPLE 15.02

### N-[6-(2-Ethyl-2H-pyrazol-3-yl)-2,4-dioxo-7-trifluoromethyl-1,4-dihydro-2H-quinazolin-3-yl]-N-propionyl-methanesulfonamide

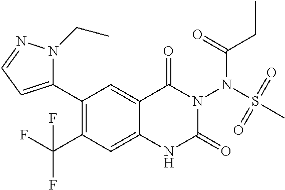 [SMILES: CCC(=O)N(n1c(=O)[nH]c2cc(C(F)(F)F)c(-c3ccnn3CC)cc2c1=O)S(C)(=O)=O]

Synthesis in analogy to Method A1 starting from N-[6-(2-ethyl-2H-pyrazol-3-yl)-2,4-dioxo-7-trifluoromethyl-1,4-dihydro-2H-quinazolin-3-yl]-methanesulfonamide and propionyl chloride to yield N-[6-(2-ethyl-2H-pyrazol-3-yl)-2,4-dioxo-7-trifluoromethyl-1,4-dihydro-2H-quinazolin-3-yl]-N-propionyl-methanesulfonamide. LC-MS at 254 nm; [M+H] 474; Rt 4.627 min; (LC-MS method V)

### EXAMPLE 15.03

### [6-(2-Ethyl-2H-pyrazol-3-yl)-2,4-dioxo-7-trifluoromethyl-1,4-dihydro-2H-quinazolin-3-yl]-methanesulfonyl-carbamic acid butyl ester

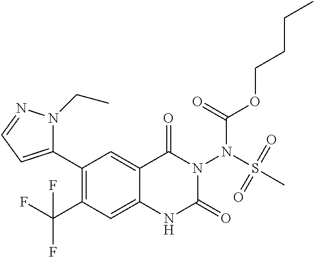 [SMILES: CCCCOC(=O)N(n1c(=O)[nH]c2cc(C(F)(F)F)c(-c3ccnn3CC)cc2c1=O)S(C)(=O)=O]

Synthesis in analogy to Method A1 starting from N-[6-(2-ethyl-2H-pyrazol-3-yl)-2,4-dioxo-7-trifluoromethyl-1,4-dihydro-2H-quinazolin-3-yl]-methanesulfonamide and butyl chloroformate to yield [6-(2-ethyl-2H-pyrazol-3-yl)-2,4-dioxo-7-trifluoromethyl-1,4-dihydro-2H-quinazolin-3-yl]-methanesulfonyl-carbamic acid butyl ester. LC-MS at 254 nm; [M+H] 518; Rt 5.304 min;

(LC-MS method V)

### EXAMPLE 15.04

### N-(2,2-Dimethyl-propionyl)-N-[6-(2-ethyl-2H-pyrazol-3-yl)-2,4-dioxo-7-trifluoromethyl-1,4-dihydro-2H-quinazolin-3-yl]-methanesulfonamide

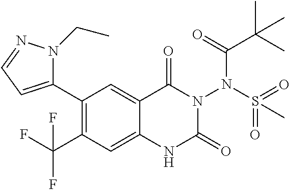 [SMILES: CCn1nccc1-c1cc2c(=O)n(N(C(=O)C(C)(C)C)S(C)(=O)=O)c(=O)[nH]c2cc1C(F)(F)F]

Synthesis in analogy to Method A1 starting from N-[6-(2-ethyl-2H-pyrazol-3-yl)-2,4-dioxo-7-trifluoromethyl-1,4-dihydro-2H-quinazolin-3-yl]-methanesulfonamide and 2,2-dimethyl-propionyl chloride to yield N-(2,2-dimethyl-propionyl)-N-[6-(2-ethyl-2H-pyrazol-3-yl)-2,4-dioxo-7-trifluoromethyl-1,4-dihydro-2H-quinazolin-3-yl]-methanesulfonamide. LC-MS at 254 nm; [M+H] 502; Rt 1.110 min; (LC-MS method VI

### Table 2: In-Vitro Activity of AMPA Receptor Antagonists (Parent Compounds)

AMPA-receptor binding can be demonstrated in standard tests, e.g. the [³H] CNQX binding test (Honore et al. Biochem. Pharmacol. 1989, 38: 3207-3212). This test is performed as follows.

Brain membranes: The animals are sacrificed, the brain removed and homogenized in 10 volumes of ice-cold 10% sucrose with a glass/Teflon homogenizer at positions 5 for 30 sec. The membranes are centrifuged at 1000×g for 10 min, and the supernatant centrifuged at 20,000×g for 15 min. The resulting pellet is resuspended in 10 volumes of cold water with a tissue homogenizer (Brinkman Polytron) at position 5 for 15 sec and the suspension centrifuged at 8000×g for 10 min. The supernatant including the buffy layer is centrifuged at 40,000×g for 20 min, the pellet resuspended in 5 volumes of water and the suspension frozen (20-30 min in dry ice/MeOH) and thawed (water-bath at 37° C.) twice. The suspension is centrifuged at 40,000×g for 20 min, the pellet resuspended in 50 mM HEPES/KOH, pH 7.5, and centrifuged at 40,000×g for 10 min. The final pellet is resuspended with a glass/Teflon homogenizer in 5 volumes of HEPES/KOH buffer; 2 mL aliquots are frozen and stored in liquid nitrogen.

Pretreatment of membranes: Membranes are thawed at 35° C. and once washed with 50 mM HEPES/KOH by centrifugation at 39,000×g for 10 min. The final pellet is resuspended with a glass/Teflon homogenizer in the same buffer.

Radioligand binding assay: It is performed using 96-well microtiterplates in a volume of 0.3 mL of 50 mM HEPES/KOH, pH 7.2, 100 μg membrane protein, 5 nM [³H]-CNQX (NEN) and the compound to be tested. Incubation is performed at 4° C. for 40 min and the reaction is terminated by centrifugation (Sigma 4K₁₀) at 3700×g for 30 min. The pellet is washed once with cold buffer and then dissolved in 0.02 mL of the tissue solubilizer Soluene for 20 min. Two hundred μL of the scintillation fluid Microscint 20 (Packard) are added and the radioactivity is counted in a Packard Topcount scintillation counter at an efficiency of 40-45%. Nonspecific binding is defined by 10 μM CNQX. Assays are performed in triplicate.

| | [3H]CNQX IC50 [uM] AMPA |
|---|---|
| Example | receptor binding, rat |
| 1.0 | 0.208 |
| 2.0 | 0.257 |
| 3.0 | 0.189 |
| 4.0 | 0.042 |
| 5.0 | 0.087 |
| 6.0 | 0.110 |
| 7.0 | n.d. |
| 8.0 | 0.162 |
| 9.0 | 0.046 |
| 10.0 | 0.159 |
| 11.0 | 0.317 |
| 12.0 | 0.162 |
| 13.0 | 0.341 |

### Table 3: In-Vivo Activity of Parent Compounds and Prodruqs in the Murine Maximal Electro Shock Test

Compounds of the invention were tested in OF1 mice using the maximal electroshock test (MES Test) described in detail by Schmutz et al., Naunyn-Schmiedeberg's Arch Pharmacol 1990, 342, 61-66. Briefly, generalized tonic-clonic convulsions of the hind extremities were induced by passing electrical current through temporal electrodes (50 Hz, 18 mA, 0.2s). Mice treated by vehicle showed mean seizure durations of 12-14s. 30 mg/kg carbamazepine was used as a positive control; mice were classified as protected by a compound If the duration of the seizure lasted only 3 second or less. Five mice were used for each treatment condition and the percentage of protected mice was used as readout (i.e. a compound could give 0%, 20%, 40%, 60%, 80% or 100% protection). Usually, compounds of the invention or their parents were given at a dose of 50 mg/kg, p.o., 1 hour prior to induction of convulsions (i.e. “pre-treatment time −1 h”).

ED50 values (ED: effective dose) were calculated using GraphPad Prism, v4.02. after shock administration, mouse blood was collected for determination of compounds' blood exposure.

TABLE 3
In-vivo activity of parent compounds and prodrugs in the murine Maximal
Electro Shock Test (MES Test)

| | %-Protection |
|---|---|
| | 50 mg/kg, p.o. |
| Example | pre-treatment time |
| No | −1 h |
| 1.0 | 20 |
| 1.01 | 40 |
| 1.02 | 100 |
| 1.03 | 100 |
| 1.04 | 80 |
| 1.05 | 80 |
| 1.06 | 60 |
| 1.07 | 80 |
| 2.0 | 80 |
| 2.01 | 100 |
| 2.02 | 100^{b} |
| 2.03 | 100^{b} |
| 2.04 | 80^{b} |
| 2.05 | 80^{b} |
| 2.06 | 60 |
| 2.07 | 100 |
| 2.08 | 80 |
| 2.09 | 40 |
| 2.10 | 100 |
| 2.11 | 100 |
| 2.12 | 60 |
| 2.13 | 20 |
| 2.14 | 100 |
| 3.0 | 100^{c} |
| 3.01 | 100^{d} |
| 3.02 | 100^{c} |
| 3.03 | 40^{c} |
| 3.04 | 20^{c} |
| 3.05 | 60^{c} |
| 3.06 | 100^{c} |
| 3.07 | 80^{c} |
| 3.08 | 100^{c} |
| 3.09 | 100^{c} |
| 3.10 | 40^{c} |
| 3.11 | 40^{a,c} |
| 3.12 | 100 |
| 3.13 | 100 |
| 3.14 | 100 |
| 3.15 | 20^{f} |
| 3.16 | 60 |
| 3.17 | 100 |
| 3.18 | 100 |
| 3.19 | 100 |
| 3.20 | 100 |
| 3.21 | 100 |
| 3.22 | 100 |
| 3.23 | 20 |
| 3.24 | 100 |
| 3.25 | 100 |
| 3.26 | 80 |
| 3.27 | 100 |
| 3.28 | 100 |
| 3.29 | 100 |
| 3.30 | 100 |
| 3.31 | 100 |
| 3.32 | 80 |
| 3.33 | 80 |
| 3.34 | 80 |
| 3.35 | 80 |
| 3.36 | 100 |
| 3.37 | 100 |
| 3.38 | 80 |
| 4.0^{g} | 0 |
| 4.01 | 20 |
| 4.02 | 100 |
| 4.03 | 40 |
| 4.04 | 40^{a} |
| 4.05 | 80 |
| 4.06 | 20 |
| 4.07 | 40 |
| 4.08 | 20 |
| 4.09 | 40 |
| 5.0 | 80 |
| 5.01 | 60 |
| 5.02 | 20 |
| 5.03 | 40 |
| 5.04 | 20 |
| 5.05 | 60 |
| 5.06 | 60 |
| 5.07 | 40 |
| 5.08 | 100 |
| 5.09 | 40 |
| 5.10 | 20 |
| 5.11 | 40 |
| 5.12 | 40 |
| 5.13 | 40 |
| 6.0 | 80, 20^{b} |
| 6.01 | 40, 20^{b} |
| 6.02 | 40, 0^{b} |
| 6.03 | 40, 0^{b} |
| 6.04 | 20, 0^{b} |
| 6.05 | 80, 60^{b} |
| 6.06 | 60, 20^{b} |
| 6.07 | 40 |
| 7.0 | 0 |
| 7.01 | 40 |
| 7.02 | 20 |
| 7.03 | 40 |
| 7.04 | 40 |
| 8.0 | 40 |
| 8.01 | 20 |
| 8.02 | 20 |
| 8.03 | 40 |
| 8.04 | 20 |
| 8.05 | 60 |
| 9.0 | 20^{a} |
| 9.01 | 40^{a} |
| 9.02 | 20^{a} |
| 9.03 | 20 |
| 9.04 | 20^{a} |
| 10.0 | 100, 0^{d} |
| 10.01 | 80, 40^{d} |
| 10.02 | 60, 60^{d} |
| 10.03 | 100, 60^{d} |
| 10.04 | 20 |
| 10.05 | 40 |
| 10.06 | 40 |
| 10.07 | 60 |
| 10.08 | 20 |
| 10.09 | 60 |
| 10.10 | 20 |
| 10.11 | 20 |
| 11.0 | 100 |
| 11.01 | 60 |
| 11.02 | 100 |
| 11.03 | 80 |
| 11.04 | 20 |
| 11.05 | 80 |
| 11.06 | 100 |
| 11.07 | 100 |
| 11.08 | 80 |
| 11.09 | 80 |
| 11.10 | 100 |
| 11.11 | 60 |
| 11.12 | 20 |
| 11.13 | 60 |
| 11.14 | 60 |
| 11.15 | 100 |
| 12.0 | 40 |
| 12.01 | 40 |
| 12.02 | 20 |
| 12.03 | 40 |
| 12.04 | 40 |
| 12.05 | 40 |
| 13.0 | 60 |
| 13.01 | 20 |
| 13.02 | 20 |
| 13.03 | 20 |
| 14.0 | 0 |
| 14.01 | 20 |
| 15.0 | 0 |
| 15.01 | 40 |
| 15.02 | 20 |
| 15.03 | 20 |
| 15.04 | 20 |

Legend:
^{a}Pre-treatment time: −2 h;
^{b}Dose: 25 mg/kg, p.o.;
^{c}Dose: 15 mg/kg, p.o.;
^{d}Pre-treatment time: −0.5 h;
^{e}Dose: 60 mg/kg, p.o
^{f}Dose: 25 mg/kg, s.c., pre-treatment time: −4 h.
^{g}ED₅₀ 10.7 mg/kg, i.p., pre-treatment time: −0.25 h

The following 36 prodrugs were tested in the above described MES Test at a dose of up to 50 mg/kg, p.o., 1 hour prior to induction of convulsions and 0%-Protection was seen:
N-[7-Ethyl-6-(2-methyl-2H-pyrazol-3-yl)-2,4-dioxo-1,4-dihydro-2H-quinazolin-3-yl]-N-isobutyryl-methanesulfonamide;
Methanesulfonyl-[7-ethyl-6-(2-methyl-2H-pyrazol-3-yl)-2,4-dioxo-1,4-dihydro-2H-quinazolin-3-yl]carbamic acid hexyl ester;
N-Decanoyl-N-[7-isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-N-(3-methyl-butyryl)-methanesulfonamide;
N-[6-(2-Isopropyl-2H-pyrazol-3-yl)-2,4-dioxo-7-trifluoromethyl-1,4-dihydro-2H-quinazolin-3-yl]-N-pentanoyl-methanesulfonamide;
4-{[6-(2-Isopropyl-2H-pyrazol-3-yl)-2,4-dioxo-7-trifluoromethyl-1,4-dihydro-2H-quinazolin-3-yl]-methanesulfonyl-amino}-4-oxo-butyric acid ethyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid methyl ester;
Acetic acid 2-{[6-(2-isopropyl-2H-pyrazol-3-yl)-2,4-dioxo-7-trifluoromethyl-1,4-dihydro-2H-quinazolin-3-yl]-methanesulfonyl-amino}-2-oxo-ethyl ester;
N-[7-Difluoromethyl-6-(2-isopropyl-2H-pyrazol-3-yl)-2,4-dioxo-1,4-dihydro-2H-quinazolin-3-yl]-N-isobutyryl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid methyl ester;
N-[7-Difluoromethyl-6-(2-isopropyl-2H-pyrazol-3-yl)-2,4-dioxo-1,4-dihydro-2H-quinazolin-3-yl]-N-hexanoyl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid butyl ester;
[7-Difluoromethyl-6-(2-isopropyl-2H-pyrazol-3-yl)-2,4-dioxo-1,4-dihydro-2H-quinazolin-3-yl]-methanesulfonyl-carbamic acid isobutyl ester;
N—((R)-2,4-Dioxo-6-tetrahydro-fu ran-2-yl-7-trifluoromethyl-1,4-dihydro-2H-quinazolin-3-yl)-N-isobutyryl-methanesulfonamide;
Methanesulfonyl-[(R)-2,4-Dioxo-6-tetrahydro-furan-2-yl-7-trifluoromethyl-1,4-dihydro-2H-quinazolin-3-yl]carbamic acid hexyl ester;
Methanesulfonyl-[(R)-2,4-Dioxo-6-tetrahydro-furan-2-yl-7-trifluoromethyl-1,4-dihydro-2H-quinazolin-3-yl]carbamic acid pentyl ester;
Methanesulfonyl-[(R)-2,4-Dioxo-6-tetrahydro-furan-2-yl-7-trifluoromethyl-1,4-dihydro-2H-quinazolin-3-yl]carbamic acid propyl ester;
Methanesulfonyl-[(R)-2,4-Dioxo-6-tetrahydro-furan-2-yl-7-trifluoromethyl-1,4-dihydro-2H-quinazolin-3-yl]carbamic acid isopropyl ester;
N-Acetyl-N-[7-difluoromethyl-6-(2-ethyl-2H-pyrazol-3-yl)-2,4-dioxo-1,4-dihydro-2H-quinazolin-3-yl]-methanesulfonamide;
N-[7-Difluoromethyl-6-(2-ethyl-2H-pyrazol-3-yl)-2,4-dioxo-1,4-dihydro-2H-quinazolin-3-yl]-N-pentanoyl-methanesulfonamide;
N-[7-Difluoromethyl-6-(2-ethyl-2H-pyrazol-3-yl)-2,4-dioxo-1,4-dihydro-2H-quinazolin-3-yl]-N-(3-methylsulfanyl-propionyl)-methanesulfonamide;
[7-Difluoromethyl-6-(2-ethyl-2H-pyrazol-3-yl)-2,4-dioxo-1,4-dihydro-2H-quinazolin-3-yl]-methanesulfonyl-carbamic acid methyl ester;
[7-Difluoromethyl-6-(2-ethyl-2H-pyrazol-3-yl)-2,4-dioxo-1,4-dihydro-2H-quinazolin-3-yl]-methanesulfonyl-carbamic acid butyl ester;
N-[7-Difluoromethyl-6-(2-ethyl-2H-pyrazol-3-yl)-2,4-dioxo-1,4-dihydro-2H-quinazolin-3-yl]-N-propionyl-methanesulfonamide;
N-[7-Difluoromethyl-6-(2-ethyl-2H-pyrazol-3-yl)-2,4-dioxo-1,4-dihydro-2H-quinazolin-3-yl]-N-(2,2-dimethyl-propionyl)-methanesulfonamide;
N-Acetyl-N-[6-(2-ethyl-2H-pyrazol-3-yl)-2,4-dioxo-7-trifluoromethyl-1,4-dihydro-2H-quinazolin-3-yl]-methanesulfonamide;
N-[6-(2-Ethyl-2H-pyrazol-3-yl)-2,4-dioxo-7-trifluoromethyl-1,4-dihydro-2H-quinazolin-3-yl]-N-pentanoyl-methanesulfonamide;
N-[6-(2-Ethyl-2H-pyrazol-3-yl)-2,4-dioxo-7-trifluoromethyl-1,4-dihydro-2H-quinazolin-3-yl]-N-(3-methylsulfanyl-propionyl)-methanesulfonamide;
Acetic acid 2-{[6-(2-ethyl-2H-pyrazol-3-yl)-2,4-dioxo-7-trifluoromethyl-1,4-dihydro-2H-quinazolin-3-yl]-methanesulfonyl-amino}-2-oxo-ethyl ester;
Methanesulfonyl-[7-ethyl-6-(2-ethyl-2H-pyrazol-3-yl)-2,4-dioxo-1,4-dihydro-2H-quinazolin-3-yl]carbamic acid ethyl ester;
Methanesulfonyl-[7-ethyl-6-(2-ethyl-2H-pyrazol-3-yl)-2,4-dioxo-1,4-dihydro-2H-quinazolin-3-yl]carbamic acid methyl ester;
Methanesulfonyl-[7-ethyl-6-(2-ethyl-2H-pyrazol-3-yl)-2,4-dioxo-1,4-dihydro-2H-quinazolin-3-yl]carbamic acid propyl ester;
Methanesulfonyl-[7-ethyl-6-(2-ethyl-2H-pyrazol-3-yl)-2,4-dioxo-1,4-dihydro-2H-quinazolin-3-yl]carbamic acid 2-methoxy-ethyl ester;
N-[7-Ethyl-6-(2-ethyl-2H-pyrazol-3-yl)-2,4-dioxo-1,4-dihydro-2H-quinazolin-3-yl]-N-isobutyryl-methanesulfonamide;
N-Acetyl-N-[7-ethyl-6-(2-ethyl-2H-pyrazol-3-yl)-2,4-dioxo-1,4-dihydro-2H-quinazolin-3-yl]-methanesulfonamide; and
N-Butyryl-N-[7-ethyl-6-(2-ethyl-2H-pyrazol-3-yl)-2,4-dioxo-1,4-dihydro-2H-quinazolin-3-yl]-methanesulfonamide.

### Table 4: PK/PD Comparison of Prodrug Versus Parent in an In-Vivo Exposure Assay

The parent compound, Example 4.0, was quantified in blood. A calibration curve was prepared in triplicate by spiking blank mouse blood with stock solutions of the parent compound covering a dynamic range from 2 ng/ml up to 31250 ng/ml. The stability of the prodrug during sample preparation and analysis was assessed by spiking some blank mouse blood samples with the stock solution of the prodrug. All samples, including the calibration samples and the prodrug QC samples, were spiked with a structurally related internal standard, then lysed and deprotonated using acetonitrile. After centrifugation, the acetonitile supernatant was diluted with 25% water and an aliquot was injected into the LC/MS system for analysis. This solution was separated on a Synergie™ Polar RP HPLC column (Phenomenex, 50*2 mm, particle size 2.5 μm) using water+1% formic acid and Methanol/Acetonitrile (1:1, v/v)+1% formic acid in a gradient elution mode. The flow from the HPLC system was directly introduced into the ion source of a TSQ Quantum Ultra MS/MS-detector (triple quadrupole mass analyzer, Thermo Scientific) and subjected to atmospheric pressure electrospray ionization (positive mode). Multireaction monitoring based on the quasi-molecular ions and the two most intense daughter ions of the parent compound was used for the selective and sensitive analysis of the compound. The quantification of the parent compound in the blank samples spiked with the prodrug moiety allowed conclusions about the conversion of the prodrug into the parent compound during the sample preparation.

| | Mouse plasma exposure of parent |
|---|---|
| | compound and %-seizure protection |
| | following a single 50 mg/kg p.o. dose; |
| Example | 30 min after treatment, MES Test |
| Prodrug, Example 4.2 | 18 678 nM/100% |
| Parent, Example 4.0 | 3352 nM/0% |

TABLE 5
Prodrug vs Parent with faster onset of action in the MES Test

| | %-Protection | | |
|---|---|---|---|
| | 50 mg/kg, p.o. | | |
| | pre-treatment time | | |
| Example | −0.25 h | −0.5 h | −1 h |
| Example 3.1 | 40 | 100 | 100 |
| Example 3.24 | 100 | 100 | 100 |
| Example 3.0 | 0 | 100 | 100 |

### Table 6: Pharmacokinetic Properties of Prodruqs in Comparison to Parents

To investigate the pharmacokinetic properties of prodrugs in comparison to parent compound, the compounds were orally administered in cynomolgus monkeys (Macaca fascicularis). Blood samples of three individuals per treatment were collected 0.25, 0.5, 0.75, 1, 2, 3, 4, 7, and 24 hours after administration. Analyses of blood concentrations of the parent drug were performed using LC/MS/MS (liquid chromatography/tandem mass spectroscopy). Lower limit of quantification was ca. 1 μmol/mL for both Example 3.0 and Example 2.0.

All compounds were administered in solution. The vehicles were:
Example 3.01: NaOH 1N/PEG300/tritisol pH9/Water 2.5/19.5/68/10, v/v/v/v
Example 3.0: PEG300/Cremophor EUwater 15/15/70, v/v/v
Example 2.01: PEG300/Cremophor EL/water 20/10/70, v/v/v, pH 5.8
Example 2.0: PEG300/Titrisol 50 mM buffer pH 9/water/HCl 1M, 20/70/9.2/0.8, v/v/v/v

As depicted in Table 6, administration of the prodrug leads to higher peripheral blood exposure compared with administration of the parent compound. After administration of Example 3.01, the time to the maximal concentration (Tmax) of the parent compound Example 3.0 in the blood was more than halved in comparison with the Example 3.0 levels observed after administration of Example 3.0 itself. Furthermore, both the maximal concentration (Cmax) and the area under the curve for 24 h (AUC0-24 h) were strongly increased (1.5-4 fold). Latter was also observed for administration of Example 2.01. Here, Cmax and AUC0-24 h of the parent compound Example 2.0 in the blood was 1.5-1.7 fold increased in comparison with administration of Example 2.0 itself. However, Tmax was in the same range (0.5-1 h). It should be noted that the sampling protocol was optimized for detecting effects on Tmax.

TABLE 6

Pharmacokinetic parameters of
Example 3.0 after administration
of 5 mg/kg of . . .

| | Example 3.01 | Example 3.0 |
|---|---|---|
| Parameter (unit) | Mean ± SD | Mean ± SD |
| Tmax (h) | 0.83 ± 0.14 | 2.00 ± 0 |
| Cmax (pmol/mL) | 12,971 ± 5067 | 2,988 ± 24 |
| AUC0-24 h (pmol/mL * h) | 24,213 ± 9,535 | 16,232 ± 474 |

Pharmacokinetic parameters of
Example 2.0 after administration
of 1 mg/kg of . . .

| | Example 2.01 | Example 2.0 |
|---|---|---|
| Parameter (unit) | Mean ± SD | Mean ± SD |
| Tmax (h) | 0.92 ± 0.14 | 0.67 ± 0.29 |
| Cmax (pmol/mL) | 794 ± 285 | 536 ± 347 |
| AUC0-24 h (pmol/mL * h) | 1,776 ± 736 | 1,027 ± 608 |

A preferred embodiment of the invention relates to compounds of formula (I) wherein the following compounds are excluded:
N-[7-Ethyl-6-(2-methyl-2H-pyrazol-3-yl)-2,4-dioxo-1,4-dihydro-2H-quinazolin-3-yl]-N-isobutyryl-methanesulfonamide;
Methanesulfonyl-[7-ethyl-6-(2-methyl-2H-pyrazol-3-yl)-2,4-dioxo-1,4-dihydro-2H-quinazolin-3-yl]carbamic acid hexyl ester;
N-Decanoyl-N-[7-isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-N-(3-methyl-butyryl)-methanesulfonamide;
N-[6-(2-Isopropyl-2H-pyrazol-3-yl)-2,4-dioxo-7-trifluoromethyl-1,4-dihydro-2H-quinazolin-3-yl]-N-pentanoyl-methanesulfonamide;
4-{[6-(2-Isopropyl-2H-pyrazol-3-yl)-2,4-dioxo-7-trifluoromethyl-1,4-dihydro-2H-quinazolin-3-yl]-methanesulfonyl-amino}-4-oxo-butyric acid ethyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid methyl ester;
Acetic acid 2-{[6-(2-isopropyl-2H-pyrazol-3-yl)-2,4-dioxo-7-trifluoromethyl-1,4-dihydro-2H-quinazolin-3-yl]-methanesulfonyl-amino}-2-oxo-ethyl ester;
N-[7-Difluoromethyl-6-(2-isopropyl-2H-pyrazol-3-yl)-2,4-dioxo-1,4-dihydro-2H-quinazolin-3-yl]-N-isobutyryl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid methyl ester;
N-[7-Difluoromethyl-6-(2-isopropyl-2H-pyrazol-3-yl)-2,4-dioxo-1,4-dihydro-2H-quinazolin-3-yl]-N-hexanoyl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid butyl ester;
[7-Difluoromethyl-6-(2-isopropyl-2H-pyrazol-3-yl)-2,4-dioxo-1,4-dihydro-2H-quinazolin-3-yl]-methanesulfonyl-carbamic acid isobutyl ester;
N—((R)-2,4-Dioxo-6-tetrahydro-furan-2-yl-7-trifluoromethyl-1,4-dihydro-2H-quinazolin-3-yl)-N-isobutyryl-methanesulfonamide;
Methanesulfonyl-[(R)-2,4-Dioxo-6-tetrahydro-furan-2-yl-7-trifluoromethyl-1,4-dihydro-2H-quinazolin-3-yl]carbamic acid hexyl ester;
Methanesulfonyl-[(R)-2,4-Dioxo-6-tetrahydro-furan-2-yl-7-trifluoromethyl-1,4-dihydro-2H-quinazolin-3-yl]carbamic acid pentyl ester;
Methanesulfonyl-[(R)-2,4-Dioxo-6-tetrahydro-furan-2-yl-7-trifluoromethyl-1,4-dihydro-2H-quinazolin-3-yl]carbamic acid propyl ester;
Methanesulfonyl-[(R)-2,4-Dioxo-6-tetrahydro-furan-2-yl-7-trifluoromethyl-1,4-dihydro-2H-quinazolin-3-yl]carbamic acid isopropyl ester;
N-Acetyl-N-[7-difluoromethyl-6-(2-ethyl-2H-pyrazol-3-yl)-2,4-dioxo-1,4-dihydro-2H-quinazolin-3-yl]-methanesulfonamide;
N-[7-Difluoromethyl-6-(2-ethyl-2H-pyrazol-3-yl)-2,4-dioxo-1,4-dihydro-2H-quinazolin-3-yl]-N-pentanoyl-methanesulfonamide;
N-[7-Difluoromethyl-6-(2-ethyl-2H-pyrazol-3-yl)-2,4-dioxo-1,4-dihydro-2H-quinazolin-3-yl]-N-(3-methylsulfanyl-propionyl)-methanesulfonamide;
[7-Difluoromethyl-6-(2-ethyl-2H-pyrazol-3-yl)-2,4-dioxo-1,4-dihydro-2H-quinazolin-3-yl]-methanesulfonyl-carbamic acid methyl ester;
[7-Difluoromethyl-6-(2-ethyl-2H-pyrazol-3-yl)-2,4-dioxo-1,4-dihydro-2H-quinazolin-3-yl]-methanesulfonyl-carbamic acid butyl ester;
N-[7-Difluoromethyl-6-(2-ethyl-2H-pyrazol-3-yl)-2,4-dioxo-1,4-dihydro-2H-quinazolin-3-yl]-N-propionyl-methanesulfonamide;
N-[7-Difluoromethyl-6-(2-ethyl-2H-pyrazol-3-yl)-2,4-dioxo-1,4-dihydro-2H-quinazolin-3-yl]-N-(2,2-dimethyl-propionyl)-methanesulfonamide;
N-Acetyl-N-[6-(2-ethyl-2H-pyrazol-3-yl)-2,4-dioxo-7-trifluoromethyl-1,4-dihydro-2H-quinazolin-3-yl]-methanesulfonamide;
N-[6-(2-Ethyl-2H-pyrazol-3-yl)-2,4-dioxo-7-trifluoromethyl-1,4-dihydro-2H-quinazolin-3-yl]-N-pentanoyl-methanesulfonamide;
N-[6-(2-Ethyl-2H-pyrazol-3-yl)-2,4-dioxo-7-trifluoromethyl-1,4-dihydro-2H-quinazolin-3-yl]-N-(3-methylsulfanyl-propionyl)-methanesulfonamide;
Acetic acid 2-{[6-(2-ethyl-2H-pyrazol-3-yl)-2,4-dioxo-7-trifluoromethyl-1,4-dihydro-2H-quinazolin-3-yl]-methanesulfonyl-amino}-2-oxo-ethyl ester;
Methanesulfonyl-[7-ethyl-6-(2-ethyl-2H-pyrazol-3-yl)-2,4-dioxo-1,4-dihydro-2H-quinazolin-3-yl]carbamic acid ethyl ester;
Methanesulfonyl-[7-ethyl-6-(2-ethyl-2H-pyrazol-3-yl)-2,4-dioxo-1,4-dihydro-2H-quinazolin-3-yl]carbamic acid methyl ester;
Methanesulfonyl-[7-ethyl-6-(2-ethyl-2H-pyrazol-3-yl)-2,4-dioxo-1,4-dihydro-2H-quinazolin-3-yl]carbamic acid propyl ester;
Methanesulfonyl-[7-ethyl-6-(2-ethyl-2H-pyrazol-3-yl)-2,4-dioxo-1,4-dihydro-2H-quinazolin-3-yl]carbamic acid 2-methoxy-ethyl ester;
N-[7-Ethyl-6-(2-ethyl-2H-pyrazol-3-yl)-2,4-dioxo-1,4-dihydro-2H-quinazolin-3-yl]-N-isobutyryl-methanesulfonamide;
N-Acetyl-N-[7-ethyl-6-(2-ethyl-2H-pyrazol-3-yl)-2,4-dioxo-1,4-dihydro-2H-quinazolin-3-yl]-methanesulfonamide; and
N-Butyryl-N-[7-ethyl-6-(2-ethyl-2H-pyrazol-3-yl)-2,4-dioxo-1,4-dihydro-2H-quinazolin-3-yl]-methanesulfonamide.

In one embodiment, the invention provides a method of inhibiting AMPA- and/or kainate-receptor activity in a subject, wherein the method comprises administering to the subject a therapeutically effective amount of a compound of the invention.

In a further embodiment, the invention provides a method of treating a disorder or a disease in a subject mediated by AMPA- and/or kainate-receptors, wherein the method comprises administering to the subject a therapeutically effective amount of a compound of the invention. Preferably said disorder or said disease is selected from epilepsy, migraine and tinnitus.

In yet a further embodiment, the invention provides the use of a compound of the invention for the manufacture of a medicament for the treatment of a disorder or disease in a subject mediated by AMPA- and/or kainate-receptors.

In yet a further embodiment, the invention provides the use of a compound of the invention for the treatment of a disorder or disease in a subject mediated by AMPA- and/or kainate-receptors.

In yet a further embodiment, the invention provides the use of a compound of the invention for the treatment of a disorder or disease in a subject characterized by an abnormal activity of AMPA- and/or kainate-receptors. Preferably said disorder or said disease is selected from epilepsy, migraine and tinnitus.

In yet a further embodiment, the invention provides a compound selected from Example 7.0, 11.0, 14.0, 15.0, 44.0, 45.0, 46.0, 47.0, 48.0, 49.0, 50.0, 51.0 and 52.0. Said compounds are parent compounds. In said embodiment, said compounds are included in the term “compounds of the invention”.

In yet a further embodiment, the invention provides a prodrug of an AMPA receptor antagonist, wherein the AMPA receptor antagonist is a 2,4-dioxo-1,4-dihydro-2H-quinazolin-3-yl-sulfonamide-based AMPA receptor antagonist or a 1,4-dihydro-quinoxaline-2,3-dione-based AMPA receptor antagonist, and wherein the prodrug achieves at a dose of 50 mg/kg p.o. with a pre-treatment time of 1 hour at least 20% protection in the MES Test.

In said embodiment, the term “prodrug of an AMPA receptor antagonist” means a compound which is transformed in vivo into an AMPA receptor antagonist. In said embodiment, the term “AMPA receptor antagonist” preferably means a compound having an IC50-value of at least 5 μM in the [³H] CNQX binding test, more preferably an IC50-value of at least 2 μM, even more preferably of at least 1 μM. In said embodiment, the term “2,4-dioxo-1,4-dihydro-2H-quinazolin-3-yl-sulfonamide-based” relates to a compound being a 2,4-dioxo-1,4-dihydro-2H-quinazolin-3-yl-sulfonamide; said compounds are described, e.g. in WO199519346, WO2006108591 and WO2006010591. In said embodiment, the term “1,4-dihydro-quinoxaline-2,3-dione-based” relates to a compound being a 1,4-dihydro-quinoxaline-2,3-dione; said compounds are described, e.g. in WO199708155. In said embodiment, said compounds are included in the term “compounds of the invention”.

1. A 2,4-dioxo-1,4-dihydro-2H-quinazolin-3-yl-sulfonamide derivative being
(A) a compound of the formula I 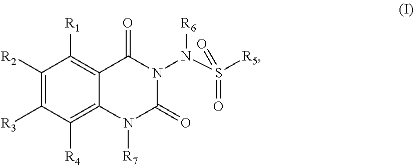
wherein
R₁ is hydrogen, halogen, C₁₋₄alkyl, C₁₋₄halogenalkyl, C₃₋₄cycloalkyl or C₃₋₄halogencycloalkyl;
R₂ is a group selected from the group consisting of 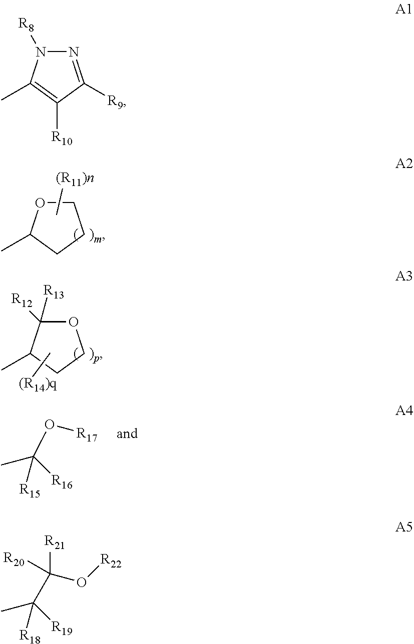
R₈ is hydrogen; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the nitrogen atom of the group A1 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the nitrogen atom of the group A1 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₉ and R₁₀ independently are hydrogen or fluoro;
m is 1 or 2;
n is 0, 1, 2 or 3;
R₁₁ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A2 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A2 or via a C₁₋₂alkylene or an oxygen, and wherein the phenyl may be substituted by halogen, hydroxy or C₁₋₄alkyl; C₁₋₆alkoxy; or C₁₋₆haloalkoxy;
p is 1 or 2;
q is 0, 1, 2 or 3;
R₁₂ is hydrogen, halogen, C₁₋₃alkyl, C₁₋₃halogenalkyl or cyclopropyl; and R₁₃ is hydrogen;
or R₁₂ and R₁₃ are independently halogen or methyl;
or R₁₂ and R₁₃ together with the carbon atom to which they are bound form a cyclopropyl;
R₁₄ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A3 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A3 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₁₅ and R₁₆ independently are hydrogen; halogen; cyano; C₁₋₆alkyl; C₁₋₆haloalkyl;
C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A4 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A4 or via a C₁₋₂alkylene or an oxygen, and wherein the phenyl may be substituted by halogen, hydroxy or C₁₋₄alkyl; C₁₋₆alkoxy; or C₁₋₆haloalkoxy;
or R₁₅ and R₁₆ together with the carbon atom to which they are bound form a C₃₋₆cycloalkyl;
R₁₇ is C₁₋₆alkyl; C₁₋₆haloalkyl; C₁₋₆hydroxyalkyl; C₁₋₄alkoxy-C₁₋₄alkyl; C₃₋₆ cycloalkyl, wherein one carbon atom may be replaced by an oxygen atom, wherein the C₃₋₆cycloalkyl may be attached directly to the oxygen atom of the group A4 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A4 or via a C₁₋₂alkylene, and wherein the phenyl may be substituted by halogen, hydroxy or C₁₋₄alkyl;
R₁₈ and R₁₉ independently are hydrogen; halogen; cyano; C₁₋₆alkyl; C₁₋₆haloalkyl;
C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A5 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A5 or via a C₁₋₂alkylene or an oxygen, and wherein the phenyl may be substituted by halogen, hydroxy or C₁₋₄alkyl; C₁₋₆alkoxy; or C₁₋₆haloalkoxy;
or R₁₈ and R₁₉ together with the carbon atom to which they are bound form a C₃₋₆cycloalkyl;
R₂₀ is hydrogen, halogen, C₁₋₃alkyl, C₁₋₃halogenalkyl or cyclopropyl; and R₂₁ is hydrogen;
or R₂₀ and R₂₁ are independently halogen or methyl;
or R₂₀ and R₂₁ together with the carbon atom to which they are bound form a cyclopropyl;
or R₁₈ and R₂₀ together with the adjacent carbon atoms to which they are bound form a C₃₋₆cycloalkyl; and R₁₉ and R₂₁ are hydrogen;
R₂₂ is C₁₋₆alkyl; C₁₋₆haloalkyl; C₁₋₆hydroxyalkyl; C₁₋₄alkoxy-C₁₋₄alkyl; C₃₋₆ cycloalkyl, wherein one carbon atom may be replaced by an oxygen atom, wherein the C₃₋₆cycloalkyl may be attached directly to the oxygen atom of the group A5 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A5 or via a C₁₋₂alkylene, and wherein the phenyl may be substituted by halogen, hydroxy or C₁₋₄alkyl;
R₃ is C₁₋₄halo genalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl;
C₂₋₄halogenalkinyl; or a three- to seven-membered monocyclic ring system which may be aromatic, saturated or unsaturated non-aromatic and which may contain from 1 to 4 hetero atoms selected from the group consisting of nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃, C₃₋₆cycloalkylcarbonyl which may be substituted once or more than once by R₂₄, phenylcarbonyl which may be substituted once or more than once by R₂₅, C₃₋₆cycloalkyl-C₁₋₂alkylcarbonyl which may be substituted once or more than once by R₂₆, phenyl-C₁₋₂alkylcarbonyl which may be substituted once or more than once by R₂₇; C₁₋₁₀alkoxycarbonyl which may be substituted once or more than once by R₂₈, or C₃₋₆cycloalkoxycarbonyl which may be substituted once or more than once by R₂₉; phenyloxycarbonyl which may be substituted once or more than once by R₃₀, C₃₋₆cycloalkyl-C₁₋₂alkoxycarbonyl which may be substituted once or more than once by R₃₁, phenyl-C₁₋₂alkoxycarbonyl which may be substituted once or more than once by R₃₂;
R₇ is hydrogen, C₁₋₁₀alkylcarbonyl which may be substituted once or more than once by R₃₃, C₃₋₆cycloalkylcarbonyl which may be substituted once or more than once by R₃₄, phenylcarbonyl which may be substituted once or more than once by R₃₅, C₃₋₆cycloalkyl-C₁₋₂alkylcarbonyl which may be substituted once or more than once by R₃₆, phenyl-C₁₋₂alkylcarbonyl which may be substituted once or more than once by R₃₇; C₁₋₁₀alkoxycarbonyl which may be substituted once or more than once by R₃₈, or C₃₋₆cycloalkoxycarbonyl which may be substituted once or more than once by R₃₉; phenyloxycarbonyl which may be substituted once or more than once by R₄₀, C₃₋₆cycloalkyl-C₁₋₂alkoxycarbonyl which may be substituted once or more than once by R₄₁, phenyl-C₁₋₂alkoxycarbonyl which may be substituted once or more than once by R₄₂;
each R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁ and R₄₂ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl;
in free form or in salt form; or
(B) a compound selected from the group consisting of
4-[(6-Imidazol-1-yl-2,4-dioxo-7-trifluoromethyl-1,4-dihydro-2H-quinazolin-3-yl)-methanesulfonyl-amino]-4-oxo-butyric acid ethyl ester;
N-(6-Imidazol-1-yl-2,4-dioxo-7-trifluoromethyl-1,4-dihydro-2H-quinazolin-3-yl)-N-pentanoyl-methanesulfonamide;
N-Acetyl-N-(6-imidazol-1-yl-2,4-dioxo-7-trifluoromethyl-1,4-dihydro-2H-quinazolin-3-yl)-methanesulfonamide;
N-(6-Imidazol-1-yl-2,4-dioxo-7-trifluoromethyl-1,4-dihydro-2H-quinazolin-3-yl)-N-propionyl-methanesulfonamide;
N-(6-Imidazol-1-yl-2,4-dioxo-7-trifluoromethyl-1,4-dihydro-2H-quinazolin-3-yl)-N-isobutyryl-methanesulfonamide;
N-(6-Imidazol-1-yl-2,4-dioxo-7-trifluoromethyl-1,4-dihydro-2H-quinazolin-3-yl)-N-(3-methyl-butyryl)-methanesulfonamide;
N-Cyclopentanecarbonyl-N-(6-imidazol-1-yl-2,4-dioxo-7-trifluoromethyl-1,4-dihydro-2H-quinazolin-3-yl)-methanesulfonamide;
N-Hexanoyl-N-(6-imidazol-1-yl-2,4-dioxo-7-trifluoromethyl-1,4-dihydro-2H-quinazolin-3-yl)-methanesulfonamide;
N-Butyryl-N-(6-imidazol-1-yl-2,4-dioxo-7-trifluoromethyl-1,4-dihydro-2H-quinazolin-3-yl)-methanesulfonamide;
Methanesulfonyl-[6-(2-methyl-imidazol-1-yl)-2,4-dioxo-7-trifluoromethyl-1,4-dihydro-2H-quinazolin-3-yl]-carbamic acid ethyl ester;
N-Butyryl-N-[6-(2-methyl-imidazol-1-yl)-2,4-dioxo-7-trifluoromethyl-1,4-dihydro-2H-quinazolin-3-yl]-methanesulfonamide;
Methanesulfonyl-[6-(2-methyl-imidazol-1-yl)-2,4-dioxo-7-trifluoromethyl-1,4-dihydro-2H-quinazolin-3-yl]-carbamic acid propyl ester;
Methanesulfonyl-[6-(2-methyl-imidazol-1-yl)-2,4-dioxo-7-trifluoromethyl-1,4-dihydro-2H-quinazolin-3-yl]-carbamic acid isobutyl ester;
N-(2,4-Dioxo-6-pyrrol-1-yl-7-trifluoromethyl-1,4-dihydro-2H-quinazolin-3-yl)-N-propionyl-methanesulfonamide;
N-Butyryl-N-(2,4-dioxo-6-pyrrol-1-yl-7-trifluoromethyl-1,4-dihydro-2H-quinazolin-3-yl)-methanesulfonamide;
Acetic acid 2-[(2,4-dioxo-6-pyrrol-1-yl-7-trifluoromethyl-1,4-dihydro-2H-quinazolin-3-yl)-methanesulfonyl-amino]-2-oxo-ethyl ester;
(2,4-Dioxo-6-pyrrol-1-yl-7-trifluoromethyl-1,4-dihydro-2H-quinazolin-3-yl)-methanesulfonyl-carbamic acid methyl ester;
(2,4-Dioxo-6-pyrrol-1-yl-7-trifluoromethyl-1,4-dihydro-2H-quinazolin-3-yl)-methanesulfonyl-carbamic acid ethyl ester;
N-(2,4-Dioxo-6-[1,2,4]triazol-4-yl-7-trifluoromethyl-1,4-dihydro-2H-quinazolin-3-yl)-N-(3-methyl-butyryl)-methanesulfonamide;
N-(2,4-Dioxo-6-[1,2,4]triazol-4-yl-7-trifluoromethyl-1,4-dihydro-2H-quinazolin-3-yl)-N-hexanoyl-methanesulfonamide;
N-(2,4-Dioxo-6-[1,2,4]triazol-4-yl-7-trifluoromethyl-1,4-dihydro-2H-quinazolin-3-yl)-N-pentanoyl-methanesulfonamide;
N-Butyryl-N-(2,4-dioxo-6-[1,2,4]triazol-4-yl-7-trifluoromethyl-1,4-dihydro-2H-quinazolin-3-yl)-methanesulfonamide;
N-Hexanoyl-N-[6-(1-hydroxy-propyl)-2,4-dioxo-7-trifluoromethyl-1,4-dihydro-2H-quinazolin-3-yl]-methanesulfonamide;
Methanesulfonyl-[6-(1-hydroxy-propyl)-2,4-dioxo-7-trifluoromethyl-1,4-dihydro-2H-quinazolin-3-yl]-carbamic acid 2-methoxy-ethyl ester;
N-Butyryl-N-[6-(1-hydroxy-propyl)-2,4-dioxo-7-trifluoromethyl-1,4-dihydro-2H-quinazolin-3-yl]-methanesulfonamide;
N-[6-(1-hydroxy-propyl)-2,4-dioxo-7-trifluoromethyl-1,4-dihydro-2H-quinazolin-3-yl]-N-isobutyryl-methanesulfonamide; and
N-[6-(1-hydroxy-propyl)-2,4-dioxo-7-trifluoromethyl-1,4-dihydro-2H-quinazolin-3-yl]-N-pentanoyl-methanesulfonamide;
in free form or in salt form.

2. A 2,4-dioxo-1,4-dihydro-2H-quinazolin-3-yl-sulfonamide derivative according to claim 1 wherein said derivative is a compound of the formula I 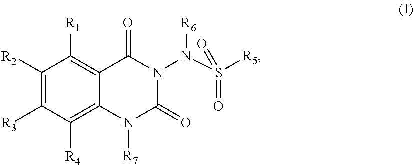
wherein
R₁ is hydrogen, halogen, C₁₋₄alkyl, C₁₋₄halogenalkyl, C₃₋₄cycloalkyl or C₃₋₄halogencycloalkyl;
R₂ is a group selected from the group consisting of 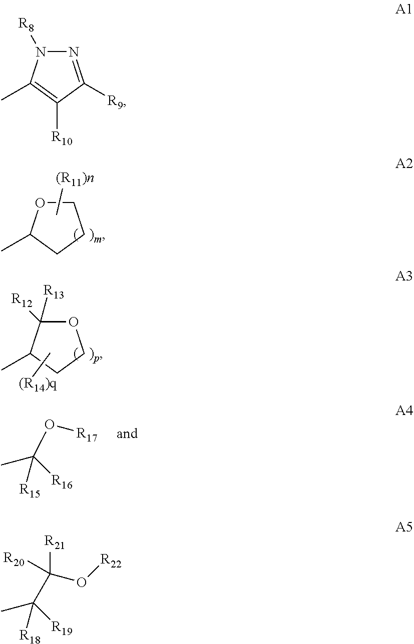
R₈ is hydrogen; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the nitrogen atom of the group A1 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the nitrogen atom of the group A1 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₉ and R₁₀ independently are hydrogen or fluoro;
m is 1 or 2;
n is 0, 1, 2 or 3;
R₁₁ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A2 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A2 or via a C₁₋₂alkylene or an oxygen, and wherein the phenyl may be substituted by halogen, hydroxy or C₁₋₄alkyl; C₁₋₆alkoxy; or C₁₋₆haloalkoxy;
p is 1 or 2;
q is 0, 1, 2 or 3;
R₁₂ is hydrogen, halogen, C₁₋₃alkyl, C₁₋₃halogenalkyl or cyclopropyl; and R₁₃ is hydrogen;
or R₁₂ and R₁₃ are independently halogen or methyl;
or R₁₂ and R₁₃ together with the carbon atom to which they are bound form a cyclopropyl;
R₁₄ is halogen; cyano; hydroxy; C₁₋₆alkyl; C₁₋₆haloalkyl; C₁₋₆hydroxyalkyl; C₁₋₄alkoxy-C₁₋₄alkyl;
C₃₋₆cycloalkyl, wherein one carbon atom may be replaced by an oxygen atom, wherein the C₃₋₆cycloalkyl may be attached directly to the carbon atom of the group A3 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A3 or via a C₁₋₂alkylene or an oxygen, and wherein the phenyl may be substituted by halogen, hydroxy or C₁₋₄alkyl; C₁₋₆alkoxy; or C₁₋₆haloalkoxy;
R₁₅ and R₁₆ independently are hydrogen; halogen; cyano; C₁₋₆alkyl; C₁₋₆haloalkyl;
C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A4 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A4 or via a C₁₋₂alkylene or an oxygen, and wherein the phenyl may be substituted by halogen, hydroxy or C₁₋₄alkyl; C₁₋₆alkoxy; or C₁₋₆haloalkoxy;
or R₁₅ and R₁₆ together with the carbon atom to which they are bound form a C₃₋₆cycloalkyl;
R₁₇ is C₁₋₆alkyl; C₁₋₆haloalkyl; C₁₋₆hydroxyalkyl; C₁₋₄alkoxy-C₁₋₄alkyl; C₃₋₆ cycloalkyl, wherein one carbon atom may be replaced by an oxygen atom, wherein the C₃₋₆cycloalkyl may be attached directly to the oxygen atom of the group A4 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A4 or via a C₁₋₂alkylene, and wherein the phenyl may be substituted by halogen, hydroxy or C₁₋₄alkyl;
R₁₈ and R₁₉ independently are hydrogen; halogen; cyano; C₁₋₆alkyl; C₁₋₆haloalkyl;
C₁₋₆hydroxyalkyl; C₁₋₄alkoxy-C₁₋₄alkyl; C₃₋₆cycloalkyl, wherein one carbon atom may be replaced by an oxygen atom, wherein the C₃₋₆cycloalkyl may be attached directly to the carbon atom of the group A5 or via a C₁₋₂alkylene or an oxygen, and wherein the C₃₋₆cycloalkyl may be substituted by halogen, hydroxy or C₁₋₄alkyl; phenyl, wherein the phenyl may be attached directly to the carbon atom of the group A5 or via a C₁₋₂alkylene or an oxygen, and wherein the phenyl may be substituted by halogen, hydroxy or C₁₋₄alkyl; C₁₋₆alkoxy; or C₁₋₆haloalkoxy;
or R₁₈ and R₁₉ together with the carbon atom to which they are bound form a C₃₋₆cycloalkyl;
R₂₀ is hydrogen, halogen, C₁₋₃alkyl, C₁₋₃halogenalkyl or cyclopropyl; and R₂₁ is hydrogen;
or R₂₀ and R₂₁ are independently halogen or methyl;
or R₂₀ and R₂₁ together with the carbon atom to which they are bound form a cyclopropyl;
or R₁₈ and R₂₀ together with the adjacent carbon atoms to which they are bound form a C₃₋₆cycloalkyl; and R₁₉ and R₂₁ are hydrogen;
R₂₂ is C₁₋₆alkyl; C₁₋₆haloalkyl; C₁₋₆hydroxyalkyl; C₁₋₄alkoxy-C₁₋₄alkyl; C₃₋₆ cycloalkyl, wherein one carbon atom may be replaced by an oxygen atom, wherein the C₃₋₆cycloalkyl may be attached directly to the oxygen atom of the group A5 or via a C₁₋₂alkylene, and wherein the C₃₋₆cycloalkyl may be substituted by halogen, hydroxy or C₁₋₄alkyl; or phenyl, wherein the phenyl may be attached directly to the oxygen atom of the group A5 or via a C₁₋₂alkylene, and wherein the phenyl may be substituted by halogen, hydroxy or C₁₋₄alkyl;
R₃ is C₁₋₄halogenalkyl, C₁₋₄alkyl, C₃₋₄cycloalkyl, C₃₋₄halogencycloalkyl, halogen or nitro;
R₄ is hydrogen or fluoro;
R₅ is C₁₋₄alkyl; C₁₋₄halogenalkyl; C₂₋₄alkenyl; C₂₋₄halogenalkenyl; C₂₋₄alkinyl; C₂₋₄halogenalkinyl; or a three- to seven-membered monocyclic ring system which may be aromatic, saturated or unsaturated non-aromatic and which may contain from 1 to 4 hetero atoms selected from the group consisting of nitrogen, oxygen and sulfur, wherein the ring system may contain not more than 2 oxygen atoms and not more than 2 sulfur atoms, wherein the ring system may be substituted once or more than once by C₁₋₄alkyl, C₁₋₄halogenalkyl, C₁₋₄alkoxy, C₁₋₄halogenalkoxy, halogen or cyano, wherein a substituent on a nitrogen in a heterocyclic ring system may not be halogen, and wherein the ring system may be directly attached to the sulfur atom or via a C₁₋₄alkylene group;
R₆ is C₁₋₁₀alkylcarbonyl which may be substituted once or more than once by R₂₃, C₃₋₆cycloalkylcarbonyl which may be substituted once or more than once by R₂₄, phenylcarbonyl which may be substituted once or more than once by R₂₅, C₃₋₆cycloalkyl-C₁₋₂alkylcarbonyl which may be substituted once or more than once by R₂₆, phenyl-C₁₋₂alkylcarbonyl which may be substituted once or more than once by R₂₇; C₁₋₁₀alkoxycarbonyl which may be substituted once or more than once by R₂₈, or C₃₋₆cycloalkoxycarbonyl which may be substituted once or more than once by R₂₉; phenyloxycarbonyl which may be substituted once or more than once by R₃₀;
C₃₋₆cycloalkyl-C₁₋₂alkoxycarbonyl which may be substituted once or more than once by R₃₁, phenyl-C₁₋₂alkoxycarbonyl which may be substituted once or more than once by R₃₂;
R₇ is hydrogen, C₁₋₁₀alkylcarbonyl which may be substituted once or more than once by R₃₃, C₃₋₆cycloalkylcarbonyl which may be substituted once or more than once by R₃₄, phenylcarbonyl which may be substituted once or more than once by R₃₅, C₃₋₆cycloalkyl-C₁₋₂alkylcarbonyl which may be substituted once or more than once by R₃₆, phenyl-C₁₋₂alkylcarbonyl which may be substituted once or more than once by R₃₇; C₁₋₁₀alkoxycarbonyl which may be substituted once or more than once by R₃₈, or C₃₋₆cycloalkoxycarbonyl which may be substituted once or more than once by R₃₉; phenyloxycarbonyl which may be substituted once or more than once by R₄₀;
C₃₋₆cycloalkyl-C₁₋₂alkoxycarbonyl which may be substituted once or more than once by R₄₁, phenyl-C₁₋₂alkoxycarbonyl which may be substituted once or more than once by R₄₂; each R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁ and R₄₂ independently is C₁₋₆alkoxy, C₁₋₄alkoxy-C₁₋₆alkoxy, phenoxy, phenyl-C₁₋₂alkoxy, C₁₋₆alkylthio, C₁₋₆alkoxycarbonyl, C₁₋₆alkylcarbonyloxy or morpholin-4-yl.

3. A compound of formula I according to claim 2, wherein R₁ is hydrogen.

4. A compound of formula I according to claim 2, wherein R₂ is a group A2; R₈ is C₁₋₆alkyl; and R₉ and R₁₀ independently are hydrogen or fluoro.

5. A compound of formula I according to claim 2, wherein R₃ is C₁₋₄halogenalkyl or C₁₋₄alkyl.

6. A compound of formula I according to claim 2, wherein R₅ is C₁₋₄alkyl.

7. A compound of formula I according to claim 2, wherein R₆ is C₁₋₁₀alkylcarbonyl which may be substituted once or more than once by R₂₃; R₇ is hydrogen or C₁₋₁₀alkylcarbonyl which may be substituted once or more than once by R₃₃; and each R₂₃ and R₃₃ independently is C₁₋₆alkoxy or C₁₋₆alkylthio.

8. A 2,4-dioxo-1,4-dihydro-2H-quinazolin-3-yl-sulfonamide derivative according to claim 1, wherein said 2,4-dioxo-1,4-dihydro-2H-quinazolin-3-yl-sulfonamide derivative is selected from the group consisting of
N-[6-(2-Methyl-2H-pyrazol-3-yl)-2,4-dioxo-7-trifluoromethyl-1,4-dihydro-2H-quinazolin-3-yl]-N-pentanoyl-methanesulfonamide;
N-Isobutyryl-N-[6-(2-methyl-2H-pyrazol-3-yl)-2,4-dioxo-7-trifluoromethyl-1,4-dihydro-2H-quinazolin-3-yl]-methanesulfonamide;
N-Butyryl-N-[6-(2-methyl-2H-pyrazol-3-yl)-2,4-dioxo-7-trifluoromethyl-1,4-dihydro-2H-quinazolin-3-yl]-methanesulfonamide;
N-Hexanoyl-N-[6-(2-methyl-2H-pyrazol-3-yl)-2,4-dioxo-7-trifluoromethyl-1,4-dihydro-2H-quinazolin-3-yl]-methanesulfonamide;
Methanesulfonyl-[6-(2-methyl-2H-pyrazol-3-yl)-2,4-dioxo-7-trifluoromethyl-1,4-dihydro-2H-quinazolin-3-yl]-carbamic acid methyl ester;
Methanesulfonyl-[6-(2-methyl-2H-pyrazol-3-yl)-2,4-dioxo-7-trifluoromethyl-1,4-dihydro-2H-quinazolin-3-yl]-carbamic acid isobutyl ester;
Methanesulfonyl-[6-(2-methyl-2H-pyrazol-3-yl)-2,4-dioxo-7-trifluoromethyl-1,4-dihydro-2H-quinazolin-3-yl]-carbamic acid 2-methoxy-ethyl ester;
N-Acetyl-N-[7-ethyl-6-(2-methyl-2H-pyrazol-3-yl)-2,4-dioxo-1,4-dihydro-2H-quinazolin-3-yl]-methanesulfonamide;
N-Butyryl-N-[7-Ethyl-6-(2-methyl-2H-pyrazol-3-yl)-2,4-dioxo-1,4-dihydro-2H-quinazolin-3-yl]-methanesulfonamide;
N-[7-Ethyl-6-(2-methyl-2H-pyrazol-3-yl)-2,4-dioxo-1,4-dihydro-2H-quinazolin-3-yl]-N-hexanoyl-methanesulfonamide;
Methanesulfonyl-[7-Ethyl-6-(2-methyl-2H-pyrazol-3-yl)-2,4-dioxo-1,4-dihydro-2H-quinazolin-3-yl]-carbamic acid isobutyl ester;
Methanesulfonyl-[7-Ethyl-6-(2-methyl-2H-pyrazol-3-yl)-2,4-dioxo-1,4-dihydro-2H-quinazolin-3-yl]-carbamic acid 2-methoxy-ethyl ester;
Methanesulfonyl-[7-Ethyl-6-(2-methyl-2H-pyrazol-3-yl)-2,4-dioxo-1,4-dihydro-2H-quinazolin-3-yl]-carbamic acid propyl ester;
Methanesulfonyl-[7-Ethyl-6-(2-methyl-2H-pyrazol-3-yl)-2,4-dioxo-1,4-dihydro-2H-quinazolin-3-yl]-carbamic acid methyl ester;
Methanesulfonyl-[7-Ethyl-6-(2-methyl-2H-pyrazol-3-yl)-2,4-dioxo-1,4-dihydro-2H-quinazolin-3-yl]-carbamic acid ethyl ester;
Methanesulfonyl-[7-Ethyl-6-(2-methyl-2H-pyrazol-3-yl)-2,4-dioxo-1,4-dihydro-2H-quinazolin-3-yl]-carbamic acid butyl ester;
N-[7-Ethyl-6-(2-methyl-2H-pyrazol-3-yl)-2,4-dioxo-1,4-dihydro-2H-quinazolin-3-yl]-N-propionyl-methanesulfonamide;
N-[7-Ethyl-6-(2-methyl-2H-pyrazol-3-yl)-2,4-dioxo-1,4-dihydro-2H-quinazolin-3-yl]-N-pentanoyl-methanesulfonamide;
Methanesulfonyl-[7-ethyl-6-(2-methyl-2H-pyrazol-3-yl)-2,4-dioxo-1,4-dihydro-2H-quinazolin-3-yl]-carbamic acid isopropyl ester;
Methanesulfonyl-[7-ethyl-6-(2-methyl-2H-pyrazol-3-yl)-2,4-dioxo-1,4-dihydro-2H-quinazolin-3-yl]-carbamic acid pentyl ester;
N-Acetyl-N-[1-acetyl-7-ethyl-6-(2-methyl-2H-pyrazol-3-yl)-2,4-dioxo-1,4-dihydro-2H-quinazolin-3-yl]-methanesulfonamide;
N-Acetyl-N-[7-isopropyl-6-(2-methyl-2H-pyrazol-3-yl)-2,4-dioxo-1,4-dihydro-2H-quinazolin-3-yl]-methanesulfonamide;
[7-Isopropyl-6-(2-methyl-2H-pyrazol-3-yl)-2,4-dioxo-1,4-dihydro-2H-quinazolin-3-yl]-methanesulfonyl-carbamic acid 2-methoxy-ethyl ester;
[7-Isopropyl-6-(2-methyl-2H-pyrazol-3-yl)-2,4-dioxo-1,4-dihydro-2H-quinazolin-3-yl]-methanesulfonyl-carbamic acid isobutyl ester;
3-(Isobutoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid isobutyl ester;
[7-Isopropyl-6-(2-methyl-2H-pyrazol-3-yl)-2,4-dioxo-1,4-dihydro-2H-quinazolin-3-yl]-methanesulfonyl-carbamic acid methyl ester;
4-{[7-Isopropyl-6-(2-methyl-2H-pyrazol-3-yl)-2,4-dioxo-1,4-dihydro-2H-quinazolin-3-yl]-methanesulfonyl-amino}-4-oxo-butyric acid ethyl ester;
3-{[7-Isopropyl-6-(2-methyl-2H-pyrazol-3-yl)-2,4-dioxo-1,4-dihydro-2H-quinazolin-3-yl]-methanesulfonyl-amino}-3-oxo-propionic acid ethyl ester;
5-{[7-Isopropyl-6-(2-methyl-2H-pyrazol-3-yl)-2,4-dioxo-1,4-dihydro-2H-quinazolin-3-yl]-methanesulfonyl-amino}-5-oxo-pentanoic acid ethyl ester;
N-[7-Isopropyl-6-(2-methyl-2H-pyrazol-3-yl)-2,4-dioxo-1,4-dihydro-2H-quinazolin-3-yl]-N-pentanoyl-methanesulfonamide;
Acetic acid 2-{[7-isopropyl-6-(2-methyl-2H-pyrazol-3-yl)-2,4-dioxo-1,4-dihydro-2H-quinazolin-3-yl]-methanesulfonyl-amino}-1,1-dimethyl-2-oxo-ethyl ester;
Acetic acid 2-[3-[(2-acetoxy-2-methyl-propionyl)-methanesulfonyl-amino]-7-isopropyl-6-(2-methyl-2H-pyrazol-3-yl)-2,4-dioxo-3,4-dihydro-2H-quinazolin-1-yl]-1,1-dimethyl-2-oxo-ethyl ester;
N-[7-Isopropyl-6-(2-methyl-2H-pyrazol-3-yl)-2,4-dioxo-1,4-dihydro-2H-quinazolin-3-yl]-N-propionyl-methanesulfonamide;
N-Butyryl-N-[7-isopropyl-6-(2-methyl-2H-pyrazol-3-yl)-2,4-dioxo-1,4-dihydro-2H-quinazolin-3-yl]-methanesulfonamide;
N-Hexanoyl-N-[7-isopropyl-6-(2-methyl-2H-pyrazol-3-yl)-2,4-dioxo-1,4-dihydro-2H-quinazolin-3-yl]-methanesulfonamide;
N-Decanoyl-N-[7-isopropyl-6-(2-methyl-2H-pyrazol-3-yl)-2,4-dioxo-1,4-dihydro-2H-quinazolin-3-yl]-methanesulfonamide;
N-Isobutyryl-N-[7-isopropyl-6-(2-methyl-2H-pyrazol-3-yl)-2,4-dioxo-1,4-dihydro-2H-quinazolin-3-yl]-methanesulfonamide;
Acetic acid 2-{[7-isopropyl-6-(2-methyl-2H-pyrazol-3-yl)-2,4-dioxo-1,4-dihydro-2H-quinazolin-3-yl]-methanesulfonyl-amino}-2-oxo-ethyl ester;
N-[7-Isopropyl-6-(2-methyl-2H-pyrazol-3-yl)-2,4-dioxo-1,4-dihydro-2H-quinazolin-3-yl]-N-(3-methylsulfanyl-propionyl)-methanesulfonamide;
[7-Isopropyl-6-(2-methyl-2H-pyrazol-3-yl)-2,4-dioxo-1,4-dihydro-2H-quinazolin-3-yl]-methanesulfonyl-carbamic acid ethyl ester;
[7-Isopropyl-6-(2-methyl-2H-pyrazol-3-yl)-2,4-dioxo-1,4-dihydro-2H-quinazolin-3-yl]-methanesulfonyl-carbamic acid propyl ester;
[7-Isopropyl-6-(2-methyl-2H-pyrazol-3-yl)-2,4-dioxo-1,4-dihydro-2H-quinazolin-3-yl]-methanesulfonyl-carbamic acid butyl ester;
[7-Isopropyl-6-(2-methyl-2H-pyrazol-3-yl)-2,4-dioxo-1,4-dihydro-2H-quinazolin-3-yl]-methanesulfonyl-carbamic acid hexyl ester;
N-(2,2-Dimethyl-propionyl)-N-[7-isopropyl-6-(2-methyl-2H-pyrazol-3-yl)-2,4-dioxo-1,4-dihydro-2H-quinazolin-3-yl]-methanesulfonamide;
N-Acetyl-N-[1-acetyl-7-isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1-propionyl-1,4-dihydro-2H-quinazolin-3-yl]-N-propionyl-methanesulfonamide;
N-Butyryl-N-[1-butyryl-7-isopropyl-6-(2-methyl-2H-pyrazol-3-yl)-2,4-dioxo-1,4-dihydro-2H-quinazolin-3-yl]-methanesulfonamide;
[1-Acetyl-7-isopropyl-6-(2-methyl-2H-pyrazol-3-yl)-2,4-dioxo-1,4-dihydro-2H-quinazolin-3-yl]-methanesulfonyl-carbamic acid benzyl ester;
N-[7-Isopropyl-6-(2-methyl-2H-pyrazol-3-yl)-2,4-dioxo-1,4-dihydro-2H-quinazolin-3-yl]-N-{2-[2-(2-methoxy-ethoxy)-ethoxy]-acetyl}-methane sulfonamide;
N-(2-Benzyloxy-acetyl)-N-[7-isopropyl-6-(2-methyl-2H-pyrazol-3-yl)-2,4-dioxo-1,4-dihydro-2H-quinazolin-3-yl]-methanesulfonamide;
N-(4-Benzyloxy-butyryl)-N-[7-isopropyl-6-(2-methyl-2H-pyrazol-3-yl)-2,4-dioxo-1,4-dihydro-2H-quinazolin-3-yl]-methanesulfonamide;
N-[7-Isopropyl-6-(2-methyl-2H-pyrazol-3-yl)-2,4-dioxo-1,4-dihydro-2H-quinazolin-3-yl]-N-(2-morpholin-4-yl-acetyl)-methanesulfonamide;
7-Isopropyl-3-(methoxycarbonyl-methanesulfonyl-amino)-6-(2-methyl-2H-pyrazol-3-yl)-2,4-dioxo-3,4-dihydro-2H-quinazoline-1-carboxylic acid methyl ester;
3-(Ethoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid ethyl ester;
7-Isopropyl-3-(methanesulfonyl-propoxycarbonyl-amino)-6-(2-methyl-2H-pyrazol-3-yl)-2,4-dioxo-3,4-dihydro-2H-quinazoline-1-carboxylic acid propyl ester;
3-(Butoxycarbonyl-methanesulfonyl-amino)-7-isopropyl-6-(2-methyl-2H-pyrazol-3-yl)-2,4-dioxo-3,4-dihydro-2H-quinazoline-1-carboxylic acid butyl ester;
3-(Acetyl-methanesulfonyl-amino)-7-isopropyl-6-(2-methyl-2H-pyrazol-3-yl)-2,4-dioxo-3,4-dihydro-2H-quinazoline-1-carboxylic acid methyl ester;
N-[7-Isopropyl-6-(2-methyl-2H-pyrazol-3-yl)-2,4-dioxo-1,4-dihydro-2H-quinazolin-3-yl]-N-(4-morpholin-4-yl-butyryl)-methanesulfonamide;
N-[7-Isopropyl-6-(2-methyl-2H-pyrazol-3-yl)-2,4-dioxo-1,4-dihydro-2H-quinazolin-3-yl]-N-(3-morpholin-4-yl-propionyl)-methanesulfonamide;
4-[(6-Imidazol-1-yl-2,4-dioxo-7-trifluoromethyl-1,4-dihydro-2H-quinazolin-3-yl)-methanesulfonyl-amino]-4-oxo-butyric acid ethyl ester;
N-(6-Imidazol-1-yl-2,4-dioxo-7-trifluoromethyl-1,4-dihydro-2H-quinazolin-3-yl)-N-pentanoyl-methanesulfonamide;
N-Acetyl-N-(6-imidazol-1-yl-2,4-dioxo-7-trifluoromethyl-1,4-dihydro-2H-quinazolin-3-yl)-methanesulfonamide;
N-(6-Imidazol-1-yl-2,4-dioxo-7-trifluoromethyl-1,4-dihydro-2H-quinazolin-3-yl)-N-propionyl-methanesulfonamide;
N-(6-Imidazol-1-yl-2,4-dioxo-7-trifluoromethyl-1,4-dihydro-2H-quinazolin-3-yl)-N-isobutyryl-methanesulfonamide;
N-(6-Imidazol-1-yl-2,4-dioxo-7-trifluoromethyl-1,4-dihydro-2H-quinazolin-3-yl)-N-(3-methyl-butyryl)-methanesulfonamide;
N-Cyclopentanecarbonyl-N-(6-imidazol-1-yl-2,4-dioxo-7-trifluoromethyl-1,4-dihydro-2H-quinazolin-3-yl)-methanesulfonamide;
N-Hexanoyl-N-(6-imidazol-1-yl-2,4-dioxo-7-trifluoromethyl-1,4-dihydro-2H-quinazolin-3-yl)-methanesulfonamide;
N-Butyryl-N-(6-imidazol-1-yl-2,4-dioxo-7-trifluoromethyl-1,4-dihydro-2H-quinazolin-3-yl)-methanesulfonamide;
N-Acetyl-N-[6-(2-isopropyl-2H-pyrazol-3-yl)-2,4-dioxo-7-trifluoromethyl-1,4-dihydro-2H-quinazolin-3-yl]-methanesulfonamide;
N-[6-(2-Isopropyl-2H-pyrazol-3-yl)-2,4-dioxo-7-trifluoromethyl-1,4-dihydro-2H-quinazolin-3-yl]-N-propionyl-methanesulfonamide;
N-Butyryl-N-[6-(2-isopropyl-2H-pyrazol-3-yl)-2,4-dioxo-7-trifluoromethyl-1,4-dihydro-2H-quinazolin-3-yl]-methanesulfonamide;
N-Hexanoyl-N-[6-(2-isopropyl-2H-pyrazol-3-yl)-2,4-dioxo-7-trifluoromethyl-1,4-dihydro-2H-quinazolin-3-yl]-methanesulfonamide;
N-Isobutyryl-N-[6-(2-isopropyl-2H-pyrazol-3-yl)-2,4-dioxo-7-trifluoromethyl-1,4-dihydro-2H-quinazolin-3-yl]-methanesulfonamide;
[6-(2-Isopropyl-2H-pyrazol-3-yl)-2,4-dioxo-7-trifluoromethyl-1,4-dihydro-2H-quinazolin-3-yl]-methanesulfonyl-carbamic acid ethyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid propyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid butyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid isobutyl ester;
[6-(2-Isopropyl-2H-pyrazol-3-yl)-2,4-dioxo-7-trifluoromethyl-1,4-dihydro-2H-quinazolin-3-yl]-methanesulfonyl-carbamic acid 2-methoxy-ethyl ester;
5-{[6-(2-Isopropyl-2H-pyrazol-3-yl)-2,4-dioxo-7-trifluoromethyl-1,4-dihydro-2H-quinazolin-3-yl]-methanesulfonyl-amino}-5-oxo-pentanoic acid ethyl ester;
3-{[6-(2-Isopropyl-2H-pyrazol-3-yl)-2,4-dioxo-7-trifluoromethyl-1,4-dihydro-2H-quinazolin-3-yl]-methanesulfonyl-amino}-3-oxo-propionic acid ethyl ester;
N-[6-(2-Isopropyl-2H-pyrazol-3-yl)-2,4-dioxo-7-trifluoromethyl-1,4-dihydro-2H-quinazolin-3-yl]-N-(3-methylsulfanyl-propionyl)-methanesulfonamide;
N-Acetyl-N-[7-difluoromethyl-6-(2-isopropyl-2H-pyrazol-3-yl)-2,4-dioxo-1,4-dihydro-2H-quinazolin-3-yl]-methanesulfonamide;
N-Butyryl-N-[7-difluoromethyl-6-(2-isopropyl-2H-pyrazol-3-yl)-2,4-dioxo-1,4-dihydro-2H-quinazolin-3-yl]-methanesulfonamide;
N-[7-Difluoromethyl-6-(2-isopropyl-2H-pyrazol-3-yl)-2,4-dioxo-1,4-dihydro-2H-quinazolin-3-yl]-N-pentanoyl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid propyl ester;
N-[7-Difluoromethyl-6-(2-isopropyl-2H-pyrazol-3-yl)-2,4-dioxo-1,4-dihydro-2H-quinazolin-3-yl]-N-propionyl-methanesulfonamide;
[7-Difluoromethyl-6-(2-isopropyl-2H-pyrazol-3-yl)-2,4-dioxo-1,4-dihydro-2H-quinazolin-3-yl]-methanesulfonyl-carbamic acid ethyl ester;
[7-Difluoromethyl-6-(2-isopropyl-2H-pyrazol-3-yl)-2,4-dioxo-1,4-dihydro-2H-quinazolin-3-yl]-methanesulfonyl-carbamic acid 2-methoxy-ethyl ester;
Methanesulfonyl-[6-(2-methyl-imidazol-1-yl)-2,4-dioxo-7-trifluoromethyl-1,4-dihydro-2H-quinazolin-3-yl]-carbamic acid ethyl ester;
N-Butyryl-N-[6-(2-methyl-imidazol-1-yl)-2,4-dioxo-7-trifluoromethyl-1,4-dihydro-2H-quinazolin-3-yl]-methanesulfonamide;
Methanesulfonyl-[6-(2-methyl-imidazol-1-yl)-2,4-dioxo-7-trifluoromethyl-1,4-dihydro-2H-quinazolin-3-yl]-carbamic acid propyl ester;
Methanesulfonyl-[6-(2-methyl-imidazol-1-yl)-2,4-dioxo-7-trifluoromethyl-1,4-dihydro-2H-quinazolin-3-yl]-carbamic acid isobutyl ester;
N-(2,4-Dioxo-6-pyrrol-1-yl-7-trifluoromethyl-1,4-dihydro-2H-quinazolin-3-yl)-N-propionyl-methanesulfonamide;
N-Butyryl-N-(2,4-dioxo-6-pyrrol-1-yl-7-trifluoromethyl-1,4-dihydro-2H-quinazolin-3-yl)-methanesulfonamide;
Acetic acid 2-[(2,4-dioxo-6-pyrrol-1-yl-7-trifluoromethyl-1,4-dihydro-2H-quinazolin-3-yl)-methanesulfonyl-amino]-2-oxo-ethyl ester;
(2,4-Dioxo-6-pyrrol-1-yl-7-trifluoromethyl-1,4-dihydro-2H-quinazolin-3-yl)-methanesulfonyl-carbamic acid methyl ester;
(2,4-Dioxo-6-pyrrol-1-yl-7-trifluoromethyl-1,4-dihydro-2H-quinazolin-3-yl)-methanesulfonyl-carbamic acid ethyl ester;
N-(2,4-Dioxo-6-[1,2,4]triazol-4-yl-7-trifluoromethyl-1,4-dihydro-2H-quinazolin-3-yl)-N-(3-methyl-butyryl)-methanesulfonamide;
N-(2,4-Dioxo-6-[1,2,4]triazol-4-yl-7-trifluoromethyl-1,4-dihydro-2H-quinazolin-3-yl)-N-hexanoyl-methanesulfonamide;
N-(2,4-Dioxo-6-[1,2,4]triazol-4-yl-7-trifluoromethyl-1,4-dihydro-2H-quinazolin-3-yl)-N-pentanoyl-methanesulfonamide;
N-Butyryl-N-(2,4-dioxo-6-[1,2,4]triazol-4-yl-7-trifluoromethyl-1,4-dihydro-2H-quinazolin-3-yl)-methanesulfonamide;
N-Acetyl-N-(2,4-dioxo-6-tetrahydro-furan-2-yl-7-trifluoromethyl-1,4-dihydro-2H-quinazolin-3-yl)-methanesulfonamide;
N-(2,4-dioxo-6-tetrahydro-furan-2-yl-7-trifluoromethyl-1,4-dihydro-2H-quinazolin-3-yl)-N-propionyl-methanesulfonamide;
N-Butyryl-N-(2,4-dioxo-6-tetrahydro-furan-2-yl-7-trifluoromethyl-1,4-dihydro-2H-quinazolin-3-yl)-methanesulfonamide;
Methanesulfonyl-N-(2,4-dioxo-6-tetrahydro-furan-2-yl-7-trifluoromethyl-1,4-dihydro-2H-quinazolin-3-yl)-carbamic acid methyl ester;
Methanesulfonyl-N-(2,4-dioxo-6-tetrahydro-furan-2-yl-7-trifluoromethyl-1,4-dihydro-2H-quinazolin-3-yl)-carbamic acid ethyl ester;
Methanesulfonyl-N-(2,4-dioxo-6-tetrahydro-furan-2-yl-7-trifluoromethyl-1,4-dihydro-2H-quinazolin-3-yl)-carbamic acid butyl ester;
Methanesulfonyl-N-(2,4-dioxo-6-tetrahydro-furan-2-yl-7-trifluoromethyl-1,4-dihydro-2H-quinazolin-3-yl)-carbamic acid 2-methoxy-ethyl ester;
Methanesulfonyl-N-(2,4-dioxo-6-tetrahydro-furan-2-yl-7-trifluoromethyl-1,4-dihydro-2H-quinazolin-3-yl)-carbamic acid isobutyl ester;
N-(2,4-dioxo-6-tetrahydro-furan-2-yl-7-trifluoromethyl-1,4-dihydro-2H-quinazolin-3-yl)-N-hexanoyl-methanesulfonamide;
4-[(2,4-Dioxo-6-tetrahydro-furan-2-yl-7-trifluoromethyl-1,4-dihydro-2H-quinazolin-3-yl)-methanesulfonyl-amino]-4-oxo-butyric acid ethyl ester;
N-(2,4-Dioxo-6-tetrahydro-furan-2-yl-7-trifluoromethyl-1,4-dihydro-2H-quinazolin-3-yl)-N-pentanoyl-methanesulfonamide;
Methanesulfonyl-[6-(1-methoxy-propyl)-2,4-dioxo-7-trifluoromethyl-1,4-dihydro-2H-quinazolin-3-yl]-carbamic acid 2-methoxy-ethyl ester;
N-[6-(1-Methoxy-propyl)-2,4-dioxo-7-trifluoromethyl-1,4-dihydro-2H-quinazolin-3-yl]-N-propionyl-methanesulfonamide;
Methanesulfonyl-[6-(1-methoxy-propyl)-2,4-dioxo-7-trifluoromethyl-1,4-dihydro-2H-quinazolin-3-yl]-carbamic acid ethyl ester;
Methanesulfonyl-[6-(1-methoxy-propyl)-2,4-dioxo-7-trifluoromethyl-1,4-dihydro-2H-quinazolin-3-yl]-carbamic acid propyl ester;
N-Hexanoyl-N-[6-(1-Methoxy-propyl)-2,4-dioxo-7-trifluoromethyl-1,4-dihydro-2H-quinazolin-3-yl]-methanesulfonamide;
N-Isobutyryl-N-[6-(1-methoxy-propyl)-2,4-dioxo-7-trifluoromethyl-1,4-dihydro-2H-quinazolin-3-yl]-methanesulfonamide;
N-Butyryl-N-[6-(1-methoxy-propyl)-2,4-dioxo-7-trifluoromethyl-1,4-dihydro-2H-quinazolin-3-yl]-methanesulfonamide;
N-Acetyl-N-[6-(1-methoxy-propyl)-2,4-dioxo-7-trifluoromethyl-1,4-dihydro-2H-quinazolin-3-yl]-methanesulfonamide;
Methanesulfonyl-[6-(1-methoxy-propyl)-2,4-dioxo-7-trifluoromethyl-1,4-dihydro-2H-quinazolin-3-yl]-carbamic acid methyl ester;
Methanesulfonyl-[6-(1-methoxy-propyl)-2,4-dioxo-7-trifluoromethyl-1,4-dihydro-2H-quinazolin-3-yl]-carbamic acid isobutyl ester;
Methanesulfonyl-[6-(1-methoxy-propyl)-2,4-dioxo-7-trifluoromethyl-1,4-dihydro-2H-quinazolin-3-yl]-carbamic acid butyl ester;
Methanesulfonyl-[6-(1-methoxy-propyl)-2,4-dioxo-7-trifluoromethyl-1,4-dihydro-2H-quinazolin-3-yl]-carbamic acid pentyl ester;
Methanesulfonyl-[6-(1-methoxy-propyl)-2,4-dioxo-7-trifluoromethyl-1,4-dihydro-2H-quinazolin-3-yl]-carbamic acid hexyl ester;
Methanesulfonyl-[6-(1-methoxy-propyl)-2,4-dioxo-7-trifluoromethyl-1,4-dihydro-2H-quinazolin-3-yl]-carbamic acid isopropyl ester;
N-[6-(1-methoxy-propyl)-2,4-dioxo-7-trifluoromethyl-1,4-dihydro-2H-quinazolin-3-yl]-N-pentanoyl-methanesulfonamide;
N-Hexanoyl-N-[6-(1-hydroxy-propyl)-2,4-dioxo-7-trifluoromethyl-1,4-dihydro-2H-quinazolin-3-yl]-methanesulfonamide;
Methanesulfonyl-[6-(1-hydroxy-propyl)-2,4-dioxo-7-trifluoromethyl-1,4-dihydro-2H-quinazolin-3-yl]-carbamic acid 2-methoxy-ethyl ester;
N-Butyryl-N-[6-(1-hydroxy-propyl)-2,4-dioxo-7-trifluoromethyl-1,4-dihydro-2H-quinazolin-3-yl]-methanesulfonamide;
N-[6-(1-hydroxy-propyl)-2,4-dioxo-7-trifluoromethyl-1,4-dihydro-2H-quinazolin-3-yl]-N-isobutyryl-methanesulfonamide;
N-[6-(1-hydroxy-propyl)-2,4-dioxo-7-trifluoromethyl-1,4-dihydro-2H-quinazolin-3-yl]-N-pentanoyl-methanesulfonamide;
Methanesulfonyl-[7-Ethyl-6-(2-ethyl-2H-pyrazol-3-yl)-2,4-dioxo-1,4-dihydro-2H-quinazolin-3-yl]carbamic acid isobutyl ester;
N-[7-Ethyl-6-(2-ethyl-2H-pyrazol-3-yl)-2,4-dioxo-1,4-dihydro-2H-quinazolin-3-yl]-N-hexanoyl-methanesulfonamide;
N-[7-Ethyl-6-(2-ethyl-2H-pyrazol-3-yl)-2,4-dioxo-1,4-dihydro-2H-quinazolin-3-yl]-N-propionyl-methanesulfonamide;
Acetic acid 2-{[7-difluoromethyl-6-(2-ethyl-2H-pyrazol-3-yl)-2,4-dioxo-1,4-dihydro-2H-quinazolin-3-yl]-methanesulfonyl-amino}-2-oxo-ethyl ester;
[6-(2-Ethyl-2H-pyrazol-3-yl)-2,4-dioxo-7-trifluoromethyl-1,4-dihydro-2H-quinazolin-3-yl]-methanesulfonyl-carbamic acid methyl ester;
N-[6-(2-Ethyl-2H-pyrazol-3-yl)-2,4-dioxo-7-trifluoromethyl-1,4-dihydro-2H-quinazolin-3-yl]-N-propionyl-methanesulfonamide;
[6-(2-Ethyl-2H-pyrazol-3-yl)-2,4-dioxo-7-trifluoromethyl-1,4-dihydro-2H-quinazolin-3-yl]-methanesulfonyl-carbamic acid butyl ester; and
N-(2,2-Dimethyl-propionyl)-N-[6-(2-ethyl-2H-pyrazol-3-yl)-2,4-dioxo-7-trifluoromethyl-1,4-dihydro-2H-quinazolin-3-yl]-methanesulfonamide;
and wherein said 2,4-dioxo-1,4-dihydro-2H-quinazolin-3-yl-sulfonamide derivative is in free form or in salt form.

9. A pharmaceutical composition comprising a therapeutically effective amount of a compound according to claim 1 and one or more pharmaceutically acceptable carriers.

10. A combination comprising a therapeutically effective amount of the compound according to claim 1 and one or more therapeutically active agents.

11. A method of inhibiting AMPA- and/or kainate-receptor activity in a subject in need thereof, wherein the method comprises administering to the subject a therapeutically effective amount of the compound according to claim 1.

12. A method of treating a disorder or a disease in a subject mediated by AMPA- and/or kainate-receptors in a subject in need thereof, wherein the method comprises administering to the subject a therapeutically effective amount of the compound according to claim 1.

13-15. (canceled)

16. A pharmaceutical composition comprising a therapeutically effective amount of a compound according to claim 2 and one or more pharmaceutically acceptable carriers.

17. A combination comprising a therapeutically effective amount of the compound according to claim 2 and one or more therapeutically active agents.

18. A method of inhibiting AMPA- and/or kainate-receptor activity in a subject in need thereof, wherein the method comprises administering to the subject a therapeutically effective amount of the compound according to claim 2.

19. A method of treating a disorder or a disease mediated by AMPA- and/or kainate-receptors in a subject in need thereof, wherein the method comprises administering to the subject a therapeutically effective amount of the compound according to claim 2.

20. A pharmaceutical composition comprising a therapeutically effective amount of a compound according to claim 8 and one or more pharmaceutically acceptable carriers.

21. A combination comprising a therapeutically effective amount of the compound according to claim 8 and one or more therapeutically active agents.

22. A method of inhibiting AMPA- and/or kainate-receptor activity in a subject in need thereof, wherein the method comprises administering to the subject a therapeutically effective amount of the compound according to claim 8.

23. A method of treating a disorder or a disease mediated by AMPA- and/or kainate-receptors in a subject in need thereof, wherein the method comprises administering to the subject a therapeutically effective amount of the compound according to claim 8.
